(19) United States
(12) Patent Application Publication
(10) Pub. No.: US 2023/0324402 A1
(43) Pub. Date: Oct. 12, 2023

(54) DEVICES FOR DETECTING ANTIBODIES TO CORONAVIRUS ANTIGENS AND METHODS FOR USING THEM

(71) Applicant: The Regents of the University of California, Oakland, CA (US)

(72) Inventors: Phillip L. FELGNER, Irvine, CA (US); Saahir KHAN, Long Beach, CA (US); Algimantas JASINSKAS, Irvine, CA (US); Rie NAKAJIMA, Pancho Santo Margarita, CA (US); Rafael RAMIRO DE ASSIS, Irvine, CA (US); Per Niklas HEDDE, Irvine, CA (US)

(21) Appl. No.: 17/914,149

(22) PCT Filed: Mar. 23, 2021
(86) PCT No.: PCT/US2021/023762

§ 371 Date: Sep. 23, 2022

Related U.S. Application Data
(60) Provisional application No. 62/993,610, filed on Mar. 23, 2020.

(51) Int. Cl. G01N 33/68; G01N 33/569; A61K 39/12

(52) U.S. Cl. CPC G01N 33/6854; G01N 33/56983; A61K 39/12; G01N 2333/165

(57) ABSTRACT

In alternative embodiments, provided are multiplexed systems, compositions, including products of manufacture such as arrays, microarrays and biochips, and kits, and methods, for detecting presence of a coronavirus in a sample, or to detect a coronavirus, e.g., COVID-19, infection. In alternative embodiments, provided are methods for detecting the presence of a coronavirus infection in an individual. In alternative embodiments, provided are vaccines and methods for using them to prevent, mitigate or lessen to effects of, or treat a coronavirus infection in an individual.

### RELATED APPLICATIONS

This Patent Convention Treaty (PCT) International Application claims benefit of priority of U.S. Provisional Application Ser. No. (USSN) 62/993,610 filed Mar. 23, 2020. The aforementioned application is expressly incorporated herein by reference in its entirety and for all purposes.

### STATEMENT AS TO FEDERALLY SPONSORED RESEARCH

This invention was made with government support under KL2 TR001416 awarded by the National Institutes of Health (NIH), and N66001-17-2-4023 and N66001-18-2-4015, awarded by the Defense Advanced Research Projects Agency (DARPA). The government has certain rights in the invention.

### TECHNICAL FIELD

This invention generally relates to infectious diseases and immunoassays. In alternative embodiments, provided are multiplexed systems, compositions, including products of manufacture such as arrays, microarrays and biochips, and kits, and methods, for detecting presence of a coronavirus in a sample, or to detect a coronavirus, e.g., COVID-19 infection. In alternative embodiments, provided are methods for detecting the presence of a coronavirus infection in an individual. In alternative embodiments, provided are vaccines and methods for using them to prevent, mitigate or lessen to effects of, or treat a coronavirus infection in an individual.

### BACKGROUND

The 2019 novel coronavirus strain (COVID-19) originating in Wuhan; China has high potential to become a worldwide pandemic with significant morbidity and mortality, with mortality estimates up to 2% of confirmed cases. The current case definition for confirmed COVID-19 infection relies on PCR-positive nasopharyngeal or respiratory specimens, with testing largely determined by presence of fever or respiratory symptoms in an individual at high epidemiologic risk.

However, this case definition likely underestimates the prevalence of COVID-19 infection, as individuals who develop subciinical infection that does not produce fever or respiratory symptoms are unlikely to be tested, and testing by PCR of nasopharyngeal or respiratory specimens is unlikely to be 100% sensitive in detecting subclinical infection. Therefore, the true prevalence of COVID-19 infection is currently unknown, and the sensitivity of PCR to detect infection is also unknown.

Serology can play an important role in defining both the prevalence of and sensitivity of PCR for COVID-19 infection, particularly for subclinical infection. This point is demonstrated by analogy with influenza virus, for which a meta-analysis of available literature measured the fraction of asymptomatic infections detected by PCR as approximately 16%, while the fraction of asymptomatic infections detected by seroconversion was measured as approximately 75% (Leung et al, Epidemiology, 2015). In addition, a key unexplained finding during the COVID-19 epidemic has been the low incidence of infection in children aged and younger. The seroprevalence of common human coronaviruses is known to increase throughout childhood to near 100% by adolescence (Zhou et al, BMC Infectious Diseases, 2013). These observations generate two related hypotheses: adults may have pre-existing antibodies against antigenically distinct coronaviruses that produce an ineffective humoral response to 2 COVID-19 infection (antibody-dependent enhancement as demonstrated for dengue virus), or children younger than 15 may have initially encountered a coronavirus that is more closely related to COVID-19 so are more protected against this infection immunologic imprinting or original antigenic sin as demonstrated for influenza virus). These hypotheses can be tested by a comprehensive investigation of serologic profiles against diverse coronavirus strains in COVID-19 cases and exposed controls.

One challenge in applying serology to COVID-19 is that the choice of antigen and choice of assay is less well defined for coronavirus than more well studied viruses such as influenza. However, prior approaches to serologic detection of infection with emerging coronaviruses including SARS and MERS have focused on the S1 domain of the spike (S) glycoprotein and the nucleocapsid (N) protein, which are considered the immunodominant antigens for these viruses (Agnihothram et al, Journal of Infectious Diseases, 2014). In particular, the S1 domain is strain-specific, while the N protein shows cross-reactivity across strains.

The assay methodologies used for serologic detection of coronavirus 5 infection include binding and neutralization assays. These methodologies have been shown to be well correlated (Chan et al, Journal of Clinical Virology, 2009). However, neutralization assays require viral culture, which must be performed in high-level biosafety containment units for emerging coronaviruses with high epidemic potential such as COVID-19.

### SUMMARY

In alternative embodiments, provided are products of manufacture such as multiplexed systems, arrays, microarrays and biochips, and kits, and methods, for detecting presence of a coronavirus in a sample, or to detect a coronavirus, e.g., COVID-19 infection.

In alternative embodiments, provided are products of manufacture for detecting the presence of a coronavirus in a sample, or to detect a coronavirus infection, wherein optionally the coronavirus is the strain COVID-19,

and optionally the product of manufacture is manufactured or fabricated as a biochip, a slide. an array or a microarray,

wherein affixed or attached (optionally covalently affixed or attached) onto a surface of the product of manufacture is:

(a) (1) at least one peptide or polypeptide epitope or antigen derived from a COVID-19 coronovirus or variant thereof, or a plurality of peptides or polypeptides comprising epitope or antigen derived from a COVID-19 coronovirus or variant thereof,

wherein the product of manufacture has affixed or attached thereon at least one viral nuclear antigen from the COVID-19 coronovirus or variant thereof, and optionally the at least one viral nuclear antigen is a coronavirus N nucleoprotein or coronavirus nonstructural protein 1 (nsp1),

wherein optionally the coronovirus is the COVID-19 coronovirus strain, and optionally the COVID-19 coronovirus strain is an L-type COVID-19 coronovirus strain and/or an S-type COVID-19 coronovirus strain; and

(b) (i) at least one peptide or polypeptide epitope or antigen derived from a non-COVID-19 virus,

Wherein optionally the non-COVID-19 virus is a virus of the subfamily Orthocoronavirinae, or the family Coronaviridae, or the order Nidovirales, and optionally the non-COVID-19 coronovirus is the SARS coronavirus (SARS-CoV) that causes severe acute respiratory syndrome (SARS), and/or the MERS-coronavirus (MERS-CoV) that causes Middle East respiratory syndrome (MERS),

and optionally the at least one peptide or polypeptide epitope or antigen 10 derived from a coronavirus, optionally a coronavirus COVID-19 virus, comprises or is: a coronavirus S1 and/or S2 spike protein domain; a coronavirus receptor-binding domain (RBD); and/or, a coronavirus nucleocapsid protein (NP) protein, and/or

(ii) a plurality of peptide or polypeptide epitopes or antigens derived from at least one of: a respiratory virus, wherein optionally the respiratory virus comprises an influenza, rhinovirus, a picornavirus, an adenovirus, a human respiratory syncytial virus, an orthopneumovirus, an enterovirus other than a rhinovirus, human parainfluenza, virus and/or a human metapneumovirus,

wherein optionally the product of manufacture comprises, or has affixed or attached onto one of its surfaces, at least one, several substantially most of or all of the peptide or polypeptide antigens or epitopes, or antigenic fragments thereof: as set forth in fable 1 (FIG. 3), Table 2 (FIG. 4) and/or Table 3 (FIG. 5); or the table of FIG. 25, and/or, one, several or all of SEQ ID NO:1 to SEQ ID NO:61, or any antigenic fragment thereof.

In alternative embodiments, provided are multiplexed systems comprising a product of manufacture as provided herein, and a digital fluorescent reader (optionally a digital fluorescent microscope comprising a digital camera or equivalent), and transmitting elements capable of transmitting data generated by the digital fluorescent reader and captured or read by the digital camera to a computer, data storage device or a cell phone or equivalents, and optionally the multiplexed system comprises a data analysis computer-executed program as described in FIG. 8 or FIG. 9.

In alternative embodiments, provided are methods for determining if an individual is positive or seropositive for an antibody against a coronavirus comprising: applying to or contacting a product of manufacture as provided herein, or a product of manufacture of the multiplexed system as provided herein, one or a plurality of fluid (optionally blood, tear, cerebral spinal fluid (CSF) or serum) samples from an individual; followed by detecting whether, or not, an antibody from the individual specifically binds to one or more of the peptide or polypeptide antigens or epitopes affixed or attached to the surface of the product of manufacture, and if an antibody from the individual specifically binds to one or more of the peptide or polypeptide antigens or epitopes, then it is determined that the individual is positive or seropositive for an antibody against a coronavirus.

In alternative embodiments, of the methods:

if the individual is positive or seropositive for antibodies that specifically bind to the coronavirus S1, S2 or Si and S2, and/or the coronavirus RBD domain of the coronavirus spike protein, the method comprises determining that the individual has immunity to the coronavirus; or

the results of detecting whether, or not, an antibody from the individual 15 specifically binds to one or more of the peptide or polypeptide antigens or epitopes affixed or attached to the surface of the product of manufacture comprises use of a data analysis computer-executed program as described in FIG. 8 or FIG. 9.

In alternative embodiments, provided are pharmaceutical formulations or vaccines comprising a peptide or polypeptide derived from the coronavirus S1, S2 or S1 and S2, and/or the coronavirus RBD domain of the coronavirus spike protein, wherein optionally the coronavirus is the strain COVID-19, wherein optionally the peptide or polypeptide derived from the coronavirus S1, S2 or Si and S2, and/or the coronavirus RBD domain of the coronavirus spike protein comprises or consists of an amino acid sequence as set forth in SEQ ID NO: 1, SEQ ID NO:2 or SEQ ID NO:3, or an antigenic fragment thereof.

In alternative embodiments, provided are methods for generating antibodies protective to a coronavirus comprising administering to an individual in need thereof a vaccine as provided herein.

In alternative embodiments, provided are uses of a product of manufacture as provided herein, or the multiplexed system as provided herein, for detecting the presence of a coronavirus in a sample, or to detect a coronavirus infection, wherein optionally the coronavirus is the strain COVID-19.

In alternative embodiments, provided are products of manufacture or multiplexed system for use in detecting the presence of a coronavirus in a sample, or to detect a coronavirus infection, wherein optionally the coronavirus is the strain COVID-19, wherein the product of manufacture is a product of manufacture as provided herein, or the multiplexed system is a multiplexed system as provided herein.

In alternative embodiments, provided are uses of a vaccine as provided herein, for generating antibodies protective to a coronavirus, wherein the use comprises administering the vaccine to an individual in need thereof.

In alternative embodiments, provided are vaccines for use in generating antibodies protective to a coronavirus, wherein the use comprises administering the vaccine as provided herein to an individual in need thereof.

The details of one or more exemplary embodiments of the invention are set forth in the accompanying drawings and the description below. Other features, objects, and advantages of the invention will be apparent from the description and drawings, and from the claims.

All publications, patents, patent applications cited herein are hereby expressly incorporated by reference in their entireties for all purposes.

### DESCRIPTION OF DRAWINGS

The patent or application file contains at least one drawing executed in color. Copies of this patent or patent application publication with color drawing(s) will be provided by the Office upon request and payment of the necessary fee.

The drawings set forth herein are illustrative of exemplary embodiments provided herein and are not meant to limit the scope of the invention as encompassed by the claims.

Figures are described in detail herein.

FIG. 1 schematically illustrates exemplary antigen array printing, probing, imaging and analysis, including an exemplary product of manufacture as provided herein and a method of making and using it, including an exemplary antigen microarray printing, probing and imaging of the array, and analysis of binding of antibodies to bound peptide or polypeptide antigen or epitope.

FIG. 2 illustrates data for the IgG seroreactivity of 5 serum specimens from a naïve population on an exemplary coronavirus antigen microarray as provided herein.

FIG. 3 illustrates Table 1, the content of an exemplary coronavirus antigen microarray as provided herein.

FIG. 4 illustrates Table 2, exemplary coronavirus antigens that can be affixed on an exemplary microarray as provided herein.

FIG. 5 illustrates Table 3, exemplary non-coronavirus antigens that can be affixed on an exemplary microarray as provided herein.

FIG. 6 schematically illustrates using an exemplary product of manufacture as provided herein, including probing and image acquisition from the array, uploading to the cloud (a remote site, or a remote computer), quantification and analysis of binding data for antibody binding to peptide or polypeptide antigen or epitope affixed on the exemplary product of manufacture.

FIG. 7 graphically illustrates the efficacy of the New UCI Printing Buffer comprising (containing) 0.15% Pluronic F127, 1% Trehalose, and 1×PATH Printing Buffer, which as demonstrated by the data gives optimal Mean Fluorescent Intensity (MFI), highest reproducibility, and largest and uniform spot sizes.

FIG. 8 schematically illustrates an exemplary data analysis and collating program used to analyze arrays as provided herein (a so-called “COVAM array”) and to generate the individual reports, as described for example in FIG. 8, and as discussed in further detail, below.

FIG. 9 schematically illustrates an exemplary data analysis and collating program used to analyze arrays as provided herein (a so-called “COVAM array”) and to generate the individual reports, as described for example in FIG. 9, and as discussed in further detail, below.

FIG. 10 illustrates Table 4, exemplary coronavirus antigens used in exemplary vaccines as provided herein.

FIG. 11 graphically illustrates normalized IgG reactivity of positive and negative sera on an exemplary Coronavirus Antigen Microarray as provided herein: the plot shows IgG reactivity against each antigen measured as mean fluorescence intensity (MFI) with full range (bars) and interquartile range (boxes) for convalescent sera from PCR-positive individuals (positive, red) and sera from naïve individuals prior to pandemic (negative, blue); Below the plot, the heatmap shows average reactivity for each group (white=low, black=mid, red=high); the antigen labels are color coded for respiratory virus group.

FIG. 12 graphically illustrates normalized IgA reactivity of positive and negative sera on an exemplary Coronavirus Antigen Microarray as provided herein: the plot show IgA reactivity against each antigen measured as mean fluorescence intensity (MFI) with full range (bars) and interquartile range (boxes) for convalescent sera from PCR-positive individuals (positive, red) and sera from naïve individuals prior to pandemic (negative, blue); below the plot, the heatmap shows average reactivity for each group (white=low, black=mid, red=high). The antigen labels are color coded for respiratory virus group.

FIG. 13 illustrates a table showing the area under receiver operating characteristic curves (ROC AUC) for SARS-CoV-2, SARS-CoV, and MERS antigens, as illustrated in FIG. 11 and FIG. 12, where the ROC AUC values for discrimination of positive and negative sera were derived for each individual antigen for both IgG and IgA and ranked.

FIG. 14 graphically illustrates ROC Curves for high-performing antigens, as illustrated in FIG. 11 and FIG. 12, the ROC curves showing sensitivity versus specificity for discrimination of positive and negative sera were derived for each individual high performing antigen (AUC≥0.95) for both IgG and IgA (solid blue line) and compared to no discrimination (AUC=0.5, dashed black line).

FIG. 15 graphically illustrates normalized antibody reactivity of positive and negative sera for high-Performing antigens, as illustrated in FIG. 11 and FIG. 12: IgG and IgA reactivity against each high-performing antigens (AUC≥0.95) measured as mean fluorescence intensity (MFI) for convalescent sera from PCR-positive individuals (positive, red) and sera from naive individuals prior to pandemic (negative, blue) are shown as box plots, including full range (bars), interquartile range (boxes), median (black line), and individual sera (dots) with p-values for each antigen calculated by Wilcoxon Rank Sum test.

FIG. 16A-B illustrates an exemplary Coronavirus Antigen Microarray heatmap: the heatmap shows IgG (FIG. 16A) and IgA (FIG. 16B) reactivity measured as mean fluorescence intensity across four replicates, against each antigen organized into rows color coded by virus, for sera organized into columns classified as positive (convalescent from PCR-positive individuals) or negative (prior to pandemic from naïve individuals); reactivity is represented by color (white=low, black=mid, red=high).

FIG. 17 illustrates a table showing the receiver operating characteristic area under curve (ROC AUC) for SARS- CoV-2, SARS-CoV, and MERS-CoV antigens. ROC AUC values for discrimination of positive and negative sera were derived for each individual antigen for both IgG and IgA and ranked, and high-performing antigens with ROC AUC≥0.86 are indicated above the lines, as discussed in Example 2, below.

FIG. 18 illustrates a table showing performance data for combinations of high-performing antigens. ROC AUC values and sensitivity and specificity based on Youden index for discrimination of positive and negative sera were derived for each individual antigen for both IgG and IgA and ranked, and high-performing antigens with ROC AUC≥0.86 are indicated above the lines, as discussed in Example 2, below.

FIG. 19A-B illustrates a heatmap for the exemplary coronavirus antigen microarray: the heatmap shows IgG (FIG. 19A) and IgA (FIG. 19B) reactivity measured as mean fluorescence intensity across four replicates, against each antigen organized into rows color coded by virus, for sera organized into columns classified as positive (convalescent from PCR-positive individuals) or negative (prior to pandemic from naïve individuals); reactivity is represented by color (white=low, black=mid, red=high), as discussed in Example 2, below.

FIG. 19A-B illustrates normalized IgG reactivity of positive and negative sera on coronavirus antigen microarray; the plot shows IgG reactivity against each antigen measured as mean fluorescence intensity (MFI) with full range (bars) and interquartile 25 range (boxes) for convalescent sera from PCR-positive individuals (positive, red) and sera from naïve individuals prior to pandemic (negative, blue); below the plot, the heatmap shows average reactivity for each group (white=low, black=mid, red=high); the antigen labels are color coded for respiratory virus group, as discussed in Example 2, below.

FIG. 20 illustrates normalized IgG reactivity of positive and negative sera on coronavirus antigen microarray; the plot shows IgG reactivity against each antigen measured as mean fluorescence intensity (MFI) with full range (bars) and interquartile range (boxes) for convalescent sera from PCR-positive individuals (positive, red) and sera from naïve individuals prior to pandemic (negative, blue); below the plot, the heatmap shows average reactivity for each group (white=low, black=mid, red=high); the antigen labels are color coded for respiratory virus group, as discussed in Example 2, below.

FIG. 21 illustrates normalized IgA reactivity of positive and negative sera on coronavirus antigen microarray; the plot shows IgG reactivity against each antigen measured as mean fluorescence intensity (MFI) with full range (bars) and interquartile range (boxes) for convalescent sera from PCR-positive individuals (positive, red) and sera from naïve individuals prior to pandemic (negative, blue). Below the plot, the heatmap shows average reactivity for each group (white=low, black=mid, red=high); the antigen labels are color coded for respiratory virus group, as discussed in Example 2, below.

FIG. 22 graphically illustrates ROC curves for high-performing antigens. ROC curves showing sensitivity versus specificity for discrimination of positive and negative sera were derived for each individual high performing antigen (ROC AUC≥0.95) for both IgG and IgA (solid blue line) and compared to no discrimination (ROC AUC=0.5, dashed black line), as discussed in Example 2, below.

FIG. 23 graphically illustrates normalized antibody reactivity of positive and negative sera for high-performing antigens; IgG and IgA reactivity against each high-performing antigens (ROC AUC≥0.95) measured as mean fluorescence intensity (MFI) for convalescent sera from PCR-positive individuals (positive, red) and sera from naive individuals prior to pandemic (negative, blue) are shown as box plots, including full range (bars), interquartile range (boxes), median (black line), and individual sera (dots) with p-values for each antigen calculated by Wilcoxon Rank Sum test, as discussed in Example 2, below.

FIG. 24 graphically illustrates ROC curves for high-performing combination of antigens. ROC curves showing sensitivity versus specificity for discrimination of positive and negative sera were derived for each combination of the high performing antigens for both IgG and IgA (solid blue line) and compared to no discrimination (ROC AUC =0.5, grey line), as discussed in Example 2, below.

FIG. 25 illustrates the content of an exemplary coronavirus antigen microarray as provided herein: the virus group, subtype, and strain, protein, GenBank identification where available, expression system, gene construct, and vendor source and catalog number are shown for each antigen.

Like reference symbols in the various drawings indicate like elements.

### DETAILED DESCRIPTION

In alternative embodiments, provided are products of manufacture such as arrays, microarrays and biochips, and kits, and methods, for detecting presence of a coronavirus in a sample, or to detect a coronavirus, e.g., COVID-19, infection. In alternative embodiments, products of manufacture including arrays, microarrays and biochips as provided herein comprise, or have displayed on, at least one viral nucleoprotein.

In alternative embodiments, products of manufacture including arrays, microarrays and biochips as provided herein comprise a plurality of proteins from the surfaces of respiratory viruses, including coronaviruses that cause epidemics (including COVID-19) and the common cold, in addition to other viruses that cause respiratory disease.

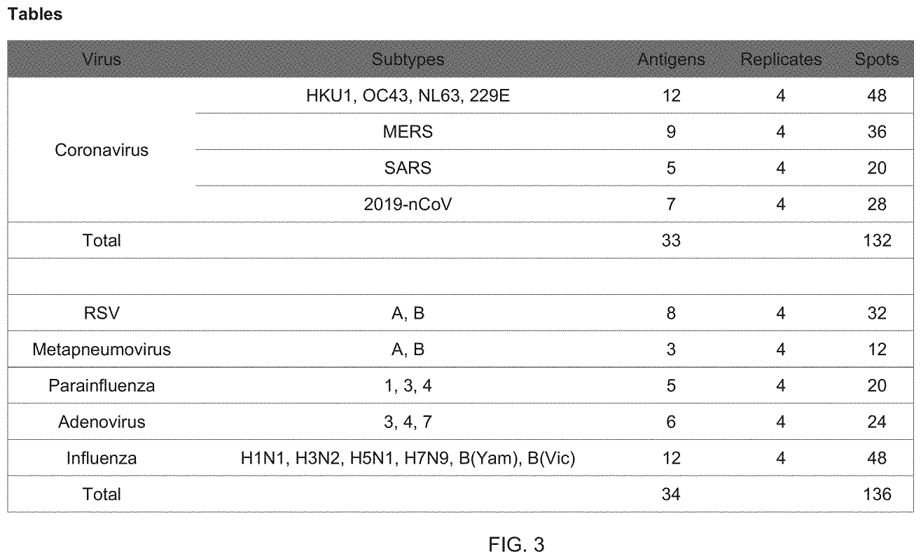

In alternative embodiments, provided are coronavirus antigen detecting devices such as biochips, arrays and microarrays that comprise multiple (or a plurality of) antigens from each of the four common coronavirus strains, and optionally further comprising antigens form SARS, MERS, and COVID-19 emerging coronavirus strains (see Table 1, or FIG. 3),

In alternative embodiments, the devices such as biochips, arrays and microarrays as provided herein also or further comprise antigens from other common respiratory viruses, for example, influenza, rhinovirus, picornavirus, adenoviruses, human respiratory syncytial virus or Orthopneumovirus, enteroviruses other than rhinovirus, human parainfluenza virus and/or human metapneumovirus, which will generate additional sero-surveillance data and serve as controls for the detection of coronavirus antibodies, e.g., for the detection of a coronavirus infection.

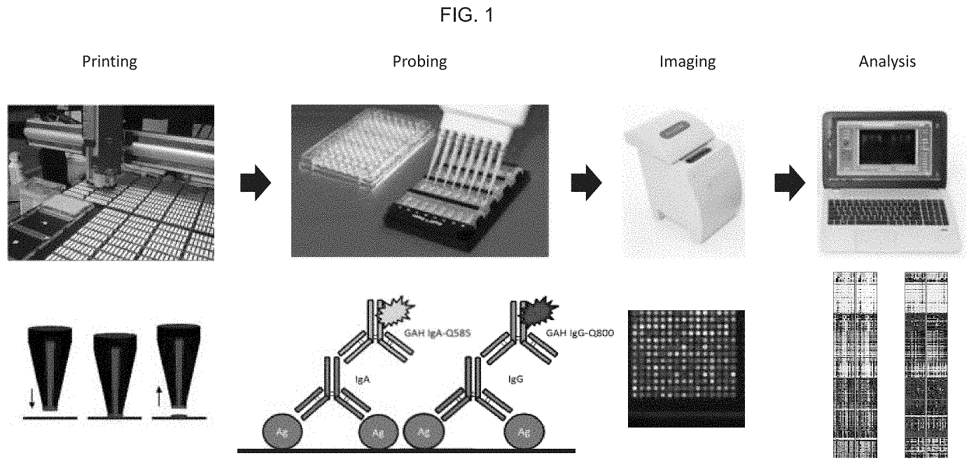

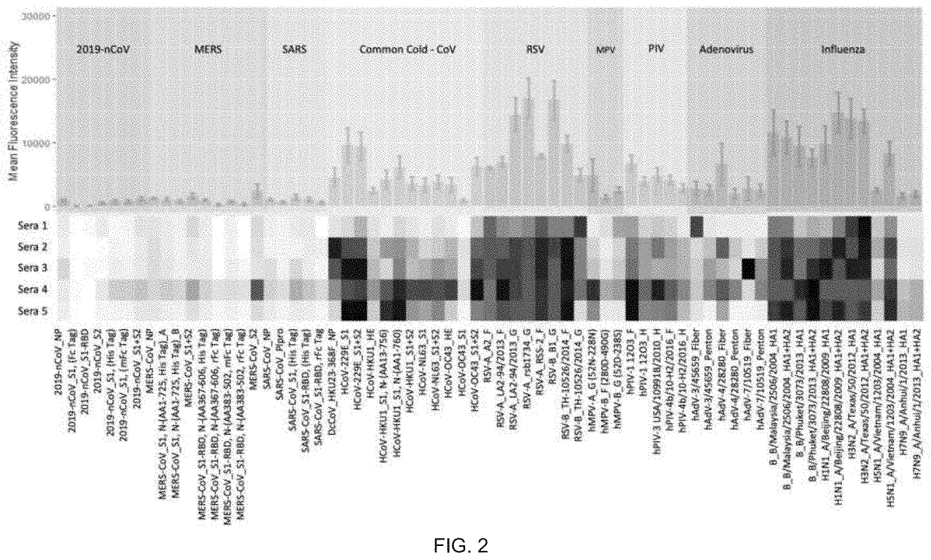

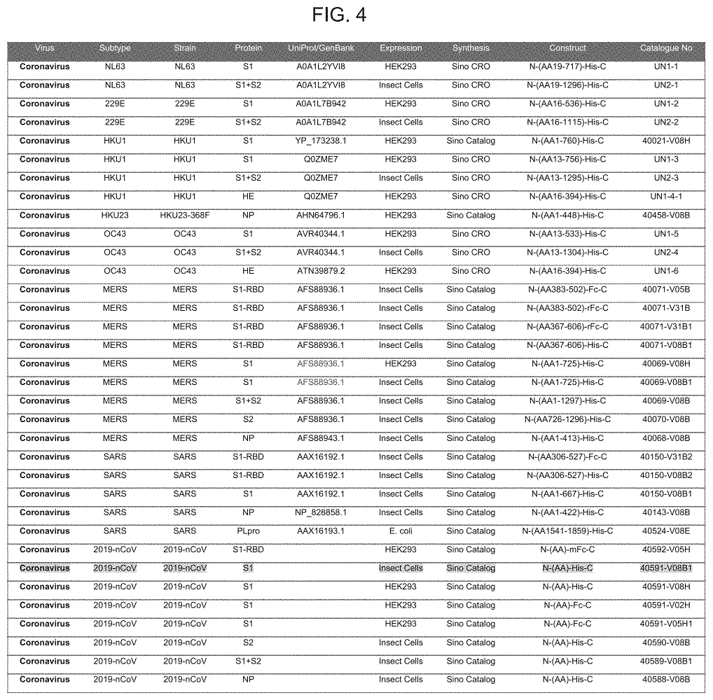

In alternative embodiments, protein antigens affixed on devices such as biochips, arrays and microarrays as provided herein are listed in Table 2, or FIG. 4. An exemplary microarray technology is schematically illustrated in FIG. 1, or FIG. 3, see also Khan S, et al. J Vis Exp (2019).

In alternative embodiments, these proteins are printed onto slides that are used to test a human serum or blood sample for antibodies against each virus. By detecting the antibodies against each virus present in human blood or serum, it can be determined (diagnosed) who has likely been infected with each virus and approximately how long ago they were infected. In addition, we can look for antibody patterns (for example, determining antibody isotype patterns) that predict immunity or more severe disease.

In alternative embodiments, products of manufacture, e.g., arrays, microarrays and biochips as provided herein are made and/or used using protein microarray methodology developed by Feigner et al, see for example WO 2006/088492 A1, and U.S. patent applications no. US 2008/0260763 A1, and US 2018/0016299 A1, which has been widely used to simultaneously perform binding assays against hundreds of antigens printed onto a nitrocellulose-coated slide for detection of multiple antibody isotypes. This methodology was recently demonstrated for simultaneous measurement of IgG and IgA antibodies against over 250 antigens from diverse strains and subtypes of influenza (Khan et al, Journal of Visualized Experiments, 2019). This methodology was previously applied to detect antibodies against the S1 domains of SARS and MFRS corona-viruses (Aeusken et al, Eurosurveillanc, 2013).

In alternative embodiments, a product of manufacture, e.g., arrays, microarrays and biochips as provided herein, and devices used to read or capture and/or transmit data related to images of fluorescent labels on the surface of a product of manufacture as provided herein, or for transmitting data related to images of fluorescent labels on the surface of a product of manufacture, can be made, manufactured or configured, or practiced (for example, using protocols and/or reagents), as described e.g., in U.S. Pat. Nos. (USPNs) 10,585,270; 10,591,485; 10,578,556; 10,451,631; 10,443,093; 10,408,759; 10,400,236; 10,180,434; 9,927,43; 9,244,076; 9,701,667; 9,139,869.

In alternative embodiments, products of manufacture, e.g., arrays, microarrays and biochips as provided herein are contacted with test solutions comprising or derived from a sample such as a liquid samples, e.g., blood, serum, sputum, or a fecal sample, taken from an individual. The product of manufacture is then read or scanned or it is otherwise determined if any antibodies from the individual specification bind to any particular peptide or polypeptide epitope or antigen bound or affixed to the product of manufacture.

In alternative embodiments, the specific binding is determined by using a secondary antibody or other moiety that specifically binds to an individual's antibodies, for example, human or animal antibodies depending on the source of the tested samples, and alternatively the secondary antibody or other moieties are directly or indirectly conjugated to or labeled with a detectable label, which alternatively can be a fluorescent label. In alternative embodiments, bound fluorescent label is detected, thereby indicating the binding, and/or the extent of binding, of an antibody in the sample to the peptide or polypeptide epitope or antigen bound or affixed to the product of manufacture, i.e., to detect the presence of an epitope or antigen binding polypeptide in the sample.

In alternative embodiments, a digital fluorescent reader, such as a digital fluorescent microscope, or equivalent (e.g., comprising a digital camera to capture the fluorescent image), is used to detect and/or measure bound fluorescent label, and the reader can create, project and/or send a high resolution image of the array and the resultant bound fluorescent signals. In alternative embodiments, the digital fluorescent reader is connected to a device such as a portable device, e.g., a cellphone or equivalent, to capture or image a high resolution image of the product of manufacture (e.g., array) and any possible bound fluorescent signals generated after applying a sample to the product of manufacture. In alternative embodiments, data representing the high resolution image of the product of manufacture with the fluorescent signals are transmitted to the cloud or a computer for data processing, quantification, and analysis. The images and results can be transmitted, e.g., emailed, back to the origin or another source.

In alternative embodiments, provided are systems, or multiplexed systems, comprising a product of manufacture as provided herein and a digital fluorescent reader, such as a digital fluorescent microscope with (or comprising) a digital camera or equivalent, and transmitting components which allow the reader to be operatively connected to or otherwise able to send data generated by the reader representing the read fluorescent images and captured by the camera or equivalent to a computer and/or a cell phone or equivalents. The digital fluorescent reader is capable of detecting and/or measuring bound fluorescent label, and the reader can create, project and/or send a high resolution image of the array and the resultant bound fluorescent signals to the computer and/or cell phone.

As described in Example 1, below, using products of manufacture as provided herein, data has been generated that yields several insights into cross-reactivity of common human coronavirus antibodies for SARS-CoV-2 antigens. The antibodies to the S1 and RBD domains of spike protein are highly subtype-specific, consistent with the high variability in these sequences between different human coronaviruses. Conversely, the coronavirus S2 domain of spike protein and coronavirus NP protein are more cross-reactive, consistent with these sequences being highly conserved across coronaviruses. Most people have pre-existing cross-reactive antibodies to coronavirus S2 domain and coronavirus NP protein, indicating that these antibodies are likely not protective, whereas antibodies to coronavirus Si and coronavirus RBD domains are more likely to be protective. This observation favors a vaccination strategy based on coronavirus S1 or coronavirus RBD domains of spike protein over a vaccination strategy that also includes S2 domain or NP protein. In addition, coronavirus S1 and RBD domains are more likely to generate subtype-specific serologic tests for population surveillance studies.

### Peptide and Protein Antigens for use on Products of Manufacture (Microarrays)

In alternative embodiments, provided are products of manufacture, including biochips, microarrays and arrays, having affixed or displayed thereon at least one peptide or polypeptide epitope or antigen derived from a COVID-19 coronovirus or variant thereof, or a plurality of peptides or polypeptides comprising epitope or antigen derived from a COVID-19 coronovirus or variant thereof, and including at least one viral nuclear antigen from the COVID-19 coronovirus or variant thereof

In alternative embodiments, the at least one viral nuclear antigen from the

COVID-19 coronovirus or variant thereof comprises SARS-CoV-2_NP (SEQ ID NO:1), or an antigenic fragment thereof:

Antigen: SARS-COV_NP, Virus: SARS-COV,
Strain: SARS, Uniprot/Genbank: NP_828858.1,
Expression: Insect, Cells, SinoCatalog: 40143-V08B
(SEQ ID NO: 1)
MSDNGPQSNQRSAPRITFGGPTDSTDNNQNGGRNGARPKQRRPQGLPNN
TASWFTALTQHGKEELRFPRGQGVPINTNSGPDDQIGYYRRATRRVRGG
DGKMKELSPRWYFYYLGTGPEASLPYGANKEGIVWVATEGALNTPKDHI
GTRNPNNNAATVLQLPQGTTLPKGFYAEGSRGGSQASSRSSSRSRGNSR
NSTPGSSRGNSPARMASGGGETALALLLLDRLNQLESKVSGKGQQQQGQ
TVTKKSAAEASKKPRQKRTATKQYNVTQAFGRRGPEQTQGNFGDQDLIR
QGTDYKHWPQIAQFAPSASAFFGMSRIGMEVTPSGTWLTYHGAIKLDDK
DPQFKDNVILLNKHIDAYKTFPPTEPKKDKKKKTDEAQPLPQRQKKQPT
VTLLPAADMDDFSRQLQNSMSGASADSTQAAHHHHHHHHHH.

In alternative embodiments; the products of manufacture, including biochips, microarrays and arrays, have affixed or displayed thereon a COVID-19 surface glycoprotein, or an antigenic fragment thereof, for example, the S1 Domain (Val16 to Arg685) is represented in bold blue and italicized characters and the RBD subdomain (Arg319 to Phe541) in bold blue and underlined characters):

(SEQ ID NO: 2)
MFVFLVLLPLVSSQCVNLTTRTQLPPAYINSFTRGVYYPDKVFRSSVLH
STQDLFLPFFSNVTWFHAIHVSGTNGTKRFDNPVLPFNDGVYFASTEKS
NIIRGWIFGTTLDSKTQSLLIVNNATNVVIKVCEFQFCNDPFLGVYYHI
KNNKSWMESEFRVYSSANNCTFEYVSQPFLMDLEGKQGNFKNLREFVFK
NIDGYFKIYSKHTPINLVRDLPQGFSALEPLVDLPIGINITRFQTLLAL
HRSYLTPGDSSSGWTAGAAAYYVGYLQPRTFLLKYNENGTITDAVDCAL
DPLSETKCTLKSFTVEKGIYQTSNFRVQPTESIVRFPNITNLCPFGEVF
NATRFASVYAWNRKRISNCVADYSVLYNSASFSTFKCYGVSPTKLNDLC
FTNYYADSFVIRGDEVRQIAPGQTGKIADYNYKTPCNGVEGFNCYFPLQ
SYGFQPTNGVGYQPYRVVVLSFELLHAPATVCGPKKSTNLVKNKCVNFN
FNGLTGTGVLTESNKKFLPFQQFGRDIADTTDAVRDPQTLEILDITPCS
FGGVSVITPGTNTSNQVAVLYQDVNCTEVPVAIHADQLTPTWRVYSTGS
NVFQTRAGCLIGAEHVNNSYECDIPIGAGICASYQTQTNSPRRARSVAS
QSIIAYTMSLGAENSVAYSNNSIAIPTNFTISVTTEILPVSMTKTSVDC
TMYICGDSTECSNLLLQYGSFCTQLNRALTGIAVEQDKNTQEVFAQVKQ
IYKTPPIKDFGGFNFSQILPDPSKPSKRSFIEDLLFNKVTLADAGFIKQ
YGDCLGDIAARDLICAQKFNGLTVLPPLLTDEMIAQYTSALLAGTITSG
WTFGAGAALQIPFAMQMAYRFNGIGVTQNVLYENQKLIANQFNSAIGKI
QDSLSSTASALGKLQDVVNQNAQALNTLVKQLSSNFGAISSVLNDILSR
LDKVEAEVQIDRLITGRLQSLQTYVTQQLIRAAEIRASANLAATKMSEC
VLGQSKRVDFCGKGYHLMSFPQSAPHGVVFLHVTYVPAQEKNFTTAPAI
CHDGKAHFPREGVFVSNGTHWFVTQRNFYEPQIITTDNTFVSGNCDVVI
GIVNNTVYDPLQPELDSFKEELDKYFKNHTSPDVDLGDISGINASVVNI
QKEIDRLNEVAKNLNESLIDLQELGKYEQYIKWPWYIWLGFIAGLIAIV
MVTIMLCCMTSCCSCLKGCCSCGSCCKFDEDDSEPVLKGVKLHYT.

In alternative embodiments, the products of manufacture, including biochips, microarrays and arrays, have affixed or displayed thereon the Si domain of the coronavirus strain COVID-19 surface glycoprotein, or an antigenic fragment thereof, where the S1 Domain stretches from Val16 until Arg685 of SEQ ID NO:2:

(SEQ ID NO: 3)
VNLTTRTQLPPAYTNSFTRGVYYPDKVFRSSVLHSTQDLFLPFFSNVTW
FHAIHVSGTNGTKRFDNPVLPFNDGVYFASTEKSNIIRGWIFGTTLDSK
TQSLLIVNNATNVVIKVCEFQFCNDPFLGVYYHKNNKSWMESEFRVYSS
ANNCTFEYVSQPFLMDLEGKQGNFKNLREFVFKNIDGYFKIYSKHTPIN
LVRDLPQGFSALEPLVDLPIGINITRFQTLLALHRSYLTPGDSSSGWTA
GAAAYYVGYLQPRTFLLKYNENGTITDAVDCALDPLSETKCTLKSFTVE
KGIYQTSNFRVQPTESIVRFPNITNLCPFGEVFNATRFASVYAWNRKRI
SNCVADYSVLYNSASFSTFKCYGVSPTKLNDLCFTNVYADSFVIRGDEV
RQIAPGQTGKIADYNYKLPDDFTGCVIAWNSNNLDSKVGGNYNYLYRLF
RKSNLKPFERDISTEIYQAGSTPCNGVEGFNCYFPLQSYGFQPTNGVGY
QPYRVVVLSFELLHAPATVCGPKKSTNLVKNKCVNFNFNGLTGTGVLTE
SNKKFLPFQQFGRDIADTTDAVRDPQTLEILDITPCSFGGVSVITPGTN
TSNQVAVLYQDVNCTEVPVAIHADQLTPTWRVYSTGSNVFQTRAGCLIG
AEHVNNSYECDIPIGAGICASYQTQTNSPRRAR.

In alternative embodiments, the products of manufacture, including biochips, microarrays and arrays, have affixed or displayed thereon the RBD subdomain of the S1 domain of the coronavirus strain COVID-19 surface glycoprotein, or an antigenic fragment thereof, where the RBD subdomain stretches from Arg319 up to Phe541 of SEQ ID NO:2:

(SEQ ID NO: 4)
RVQPTESIVRFPNITNLCPFGEVFNATRFASVYAWNRKRISNCVADYSV
LYNSASFSTFKCYGVSPTKLNDLCFTNVYADSFVIRGDEVRQIAPGQTG
KIADYNYKLPDDFTGCVIAWNSNNLDSKVGGNYNYLYRLFRKSNLKPFE
RDISTEIYQAGSTPCNGVEGFNCYFPLQSYGFQPTNGVGYQPYRVVVLS
FELLHAPATVCGPKKSTNLVKNKCVNF.

In alternative embodiments, the product of manufacture comprises, or has affixed or attached onto one of its surfaces, at least one, several substantially most of or all of the peptide or polypeptide antigens or epitopes as set forth in Table 1, Table 2 and/or Table 3, or one, several or all of the following proteins or peptides, or antigenic fragments thereof:

Antigen: DcCoV_HKU23-368F_NP, Virus: CommonCoV, Strain: HKU23-368F,
Uniprot/Genbank: AHN64796.1, Expression: HEK293, SinoCatalog: 40458-V08B
(SEQ ID NO: 5)
MSFTPGKQSSSRASSGNRSGNGILKWADQSDQSRNVQTRGRRAQPKQTVTSQ
QPSGGNVVPYYSWFSGITQFQKGKEFEFAEGQGVPIAPGVPATEAKGYWYRH
NRRSFKTADGNQRQLLPRWYFYYLGTGPHAKDQYGTDIDGVFWVASNQAD
VNTPADILDRDPSSDEAIPTRFPPGTVLPQGYYIEGSGRSAPNSRSTSRASSRAS
SAGSRSRANSGNRTPTSGVTPDMADQIASLVLAKLGKDATKPQQVTKQTAKE
IRQKILNKPRQKRSPNKQCTVQQCFGKRGPNQNFGGGEMLKLGTSDPQFPILA
ELAPTAGAFFFGSRLELAKVQNLSGNLDEPQKDVYELRYNGAIRFDSTLSGFE
TIMKVLNDNLNAYQQQDGMMNMSPKPQRQRGQKNGQGENDNISVAAPKSR
VQQNKSRELTAEDISLLKKMDEPFTENTSEIAHHHHHHHHHH
Antigen: B_B/Malaysia/2506/2004_HA1, Virus: Influenza,
Strain: B/Malaysia/2506/2004, Uniprot/Genbank: CO05957.1, Expression: HEK293,
SinoCatalog: 11716-V08H1
(SEQ ID NO: 6)
MKAIIVLLMVVTSNADRICTGITSSNSPHVVKTATQGEVNVTGVIPLTTTPTKS
HFANLKGTETRGKLCPKCLNCTDLDVALGRPKCTGNIPSARVSILHEVRPVTS
GCFPIMHDRTKIRQLPNLLRGYEHIRLSTHNVINAENAPGGPYKIGTSGSCPNV
TNGNGFFATMAWAVPKNDNNKTATNSLTIEVPYICTEGEDQITVWGFHSDNE
IQMAKLYGDSKPQKFTSSANGVTTHYVSQIGGFPNQTEDGGLPQSGRIVVDY
MVQKSGKTGTITYQRGILLPQKVWCASGRSKVIKGSLPLIGEADCLHEKYGG
LNKSKPYYTGEHAKAIGNCPIWVKTPLKLANGTKYRPPAKLLKER
Antigen: B_B/Malaysia/2506/2004_HA1 + HA2, Virus: Influenza,
Strain: B/Malaysia/2506/2004, Uniprot/Genbank: CO05957.1, Expression: HEK293,
SinoCatalog: 11716-V08H
(SEQ ID NO: 7)
MKAIIVLLMVVTSNADRICTGITSSNSPHVVKTATQGEVNVTGVIPLTTTPTKS
HFANLKGTETRGKLCPKCLNCTDLDVALGRPKCTGNIPSARVSILHEVRPVTS
GCFPIMHDRTKIRQLPNLLRGYEHIRLSTHNVINAENAPGGPYKIGTSGSCPNV
TNGNGFFATMAWAVPKNDNNKTATNSLTIEVPYICTEGEDQITVWGFHSDNE
IQMAKLYGDSKPQKFTSSANGVTTHYVSQIGGFPNQTEDGGLPQSGRIVVDY
MVQKSGKTGTITYQRGILLPQKVWCASGRSKVIKGSLPLIGEADCLHEKYGG
LNKSKPYYTGEHAKAIGNCPIWVKTPLKLANGTKYRPPAKLLKERGFFGAIA
GFLEGGWEGMIAGWHGYTSHGAHGVAVAADLKSTQEAINKITKNLNSLSEL
EVKNLQRLSGAMDELHNEILELDEKVDDLRADTISSQIELAVLLSNEGIINSED
EHLLALERKLKKMLGPSAVEIGNGCFETKHKCNQTCLDRIAAGTFDAGEFSLP
TFDSLNITAASLNDDGLDNHTILLYYSTA
Antigen: H1N1_A/Beijing/22808/2009_HA1, Virus: Influenza,
Strain: A/Beijing/22808/2009, Uniprot/Genbank: ADD64203.1, Expression: HEK293,
SinoCatalog: 40035-V08H1
(SEQ ID NO: 8)
MKAILVVLLYTFATANADTLCIGYHANNSTDTVDTVLEKNVTVTHSVNLLED
KHNGKLCKLRGVAPLHLGKCNIAGWILGNPECESLSTASSWSYIVETSSSDNG
TCYPGDFIDYEELREQLSSVSSFERFEIFPKTSSWPNHDSNKGVTAACPHAGA
KSFYKNLIWLVKKGNSYPKLSKSYINDKGKEVLVLWGIHHPSTSADQQSLYQ
NADAYVFVGTSRYSKKFKPEIAIRPKVRDQEGRMNYYWTLVEPGDKITFEAT
GNLVVPRYAFAMERNAGSGIIISDTPVHDCNTTCQTPKGAINTSLPFQNIHPITI
GKCPKYVKSTKLRLATGLRNVPSIQSRGLFGAIAGFIEGGWTGMVDGWYGY
HHQNEQGSGYAADLKSTQNAIDKITNKVNSVIEKMNTQFTAVGKEFNHLEKR
IENLNKKVDDGFLDIWTYNAELLVLLENERTLDYHDSNVKNLYEKVRSQLKN
NAKEIGNGCFEFYHKCDNTCMESVKNGTYDYPKYSEEAKLNREEIDGVKLES
TRIYQILAIYSTVASSLVLVVSLGAISFWMCSNGSLQCRICI
Antigen: H1N1_A/Beijing/22808/2009_HA1 + HA2, Virus: Influenza,
Strain: A/Beijing/22808/2009, Uniprot/Genbank: ADD64203.1, Expression: HEK293,
SinoCatalog: 40035-V08H
(SEQ ID NO: 9)
MKAILVVLLYTFATANADTLCIGYHANNSTDTVDTVLEKNVTVTHSVNLLED
KHNGKLCKLRGVAPLHLGKCNIAGWILGNPECESLSTASSWSYIVETSSSDNG
TCYPGDFIDYEELREQLSSVSSFERFEIFPKTSSWPNHDSNKGVTAACPHAGA
KSFYKNLIWLVKKGNSYPKLSKSYINDKGKEVLVLWGIHHPSTSADQQSLYQ
NADAYVFVGTSRYSKKFKPEIAIRPKVRDQEGRMNYYWTLVEPGDKITFEAT
GNLVVPRYAFAMERNAGSGIIISDTPVHDCNTTCQTPKGAINTSLPFQNIHPITI
GKCPKYVKSTKLRLATGLRNVPSIQSRGLFGAIAGFIEGGWTGMVDGWYGY
HHQNEQGSGYAADLKSTQNAIDKITNKVNSVIEKMNTQFTAVGKEFNHLEKR
IENLNKKVDDGFLDIWTYNAELLVLLENERTLDYHDSNVKNLYEKVRSQLKN
NAKEIGNGCFEFYHKCDNTCMESVKNGTYDYPKYSEEAKLNREEIDGVKLES
TRIYQILAIYSTVASSLVLVVSLGAISFWMCSNGSLQCRICI
Antigen: H3N2_A/Texas/50/2012 HA1, Virus: Influenza, Strain: A/Texas/50/2012,
Uniprot/Genbank: AGL07159.1, Expression: HEK293, SinoCatalog: 40354-V08H1
(SEQ ID NO: 10)
MKTIIALSYILCLVFAQKLPGNDNSTATLCLGHHAVPNGTIVKTITNDRIEVTN
ATELVQNSSIGEICDSPHQILDGENCTLIDALLGDPQCDGFQNKKWDLFVERS
KAYSNCYPYDVPDYASLRSLVASSGTLEFNNESFNWNGVTQNGTSSACIRRS
NNSFFSRLNWLTHLNFKYPALNVTMPNNEQFDKLYIWGVHHPGTDKDQIFLY
AQPSGRITVSTKRSQQAVIPNIGSRPRIRNIPSRISIYWTIVKPGDILLINSTGNLI
APRGYFKIRSGKSSIMRSDAPIGKCKSECITPNGSIPNDKPFQNVNRITYGACPR
YVKQSTLKLATGMRNVPEKQTRGIFGAIAGFIENGWEGMVDGWYGFRHQNS
EGRGQAADLKSTQAAIDQINGKLNRLIGKTNEKFHQIEKEFSEVEGRIQDLEK
YVEDTKIDLWSYNAELLVALENQHTIDLTDSEMNKLFEKTKKQLRENAEDM
GNGCFKIYHKCDNACIGSIRNGTYDHDVYRDEALNNRFQIKGVELKSGYKD
WILWISFAISCFLLCVALLGFIMWACQKGNIRCNICI
Antigen: H3N2_A/Texas/50/2012_HA1 + HA2, Virus: Influenza,
Strain: A/Texas/50/2012, Uniprot/Genbank: AGL07159.1, Expression: Insect, Cells,
SinoCatalog: 40354-V08B
(SEQ ID NO: 11)
MKTIIALSYILCLVFAQKLPGNDNSTATLCLGHHAVPNGTIVKTITNDRIEVTN
ATELVQNSSIGEICDSPHQILDGENCTLIDALLGDPQCDGFQNKKWDLFVERS
KAYSNCYPYDVPDYASLRSLVASSGTLEFNNESFNWNGVTQNGTSSACIRRS
NNSFFSRLNWLTHLNFKYPALNVTMPNNEQFDKLYIWGVHHPGTDKDQIFLY
AQPSGRITVSTKRSQQAVIPNIGSRPRIRNIPSRISIYWTIVKPGDILLINSTGNLI
APRGYFKIRSGKSSIMRSDAPIGKCKSECITPNGSIPNDKPFQNVNRITYGACPR
YVKQSTLKLATGMRNVPEKQTRGIFGAIAGFIENGWEGMVDGWYGFRHQNS
EGRGQAADLKSTQAAIDQINGKLNRLIGKTNEKFHQIEKEFSEVEGRIQDLEK
YVEDTKIDLWSYNAELLVALENQHTIDLTDSEMNKLFEKTKKQLRENAEDM
GNGCFKIYHKCDNACIGSIRNGTYDHDVYRDEALNNRFQIKGVELKSGYKD
WILWISFAISCFLLCVALLGFIMWACQKGNIRCNICI
Antigen: H5N1_A/Vietnam/1203/2004_HA1, Virus: Influenza,
Strain: A/Vietnam/1203/2004, Uniprot/Genbank: AAW80717.1, Expression: HEK293,
SinoCatalog: 10003-V06H1
(SEQ ID NO: 12)
MEKIVLLFAIVSLVKSDQICIGYHANNSTEQVDTIMEKNVTVTHAQDILEKKH
NGKLCDLDGVKPLILRDCSVAGWLLGNPMCDEFINVPEWSYIVEKANPVNDL
CYPGDFNDYEELKHLLSRINHFEKIQIIPKSSWSSHEASLGVSSACPYQGKSSFF
RNVVWLIKKNSTYPTIKRSYNNTNQEDLLVLWGIHHPNDAAEQTKLYQNPTT
YISVGTSTLNQRLVPRIATRSKVNGQSGRMEFFWTILKPNDAINFESNGNFIAP
EYAYKIVKKGDSTIMKSELEYGNCNTKCQTPMGAINSSMPFHNIHPLTIGECP
KYVKSNRLVLATGLRNSPQRERRRKKRGLFGAIAGFIEGGWQGMVDGWYG
YHHSNEQGSGYAADKESTQKAIDGVTNKVNSIIDKMNTQFEAVGREFNNLER
RIENLNKKMEDGFLDVWTYNAELLVLMENERTLDFHDSNVKNLYDKVRLQL
RDNAKELGNGCFEFYHKCDNECMESVRNGTYDYPQYSEEARLKREEISGVKL
ESIGIYQILSIYSTVASSLALAIMVAGLSLWMCSNGSLQCRICI
Antigen: H5N1 A/Vietnam/1203/2004 HA1 + HA2, Virus: Influenza,
Strain: A/Vietnam/1203/2004, Uniprot/Genbank: AAW80717.1, Expression: HEK293,
SinoCatalog: 10003-V06H3
(SEQ ID NO: 13)
MEKIVLLFAIVSLVKSDQICIGYHANNSTEQVDTIMEKNVTVTHAQDILEKKH
NGKLCDLDGVKPLILRDCSVAGWLLGNPMCDEFINVPEWSYIVEKANPVNDL
CYPGDFNDYEELKHLLSRINHFEKIQIIPKSSWSSHEASLGVSSACPYQGKSSFF
RNVVWLIKKNSTYPTIKRSYNNTNQEDLLVLWGIHHPNDAAEQTKLYQNPTT
YISVGTSTLNQRLVPRIATRSKVNGQSGRMEFFWTILKPNDAINFESNGNFIAP
EYAYKIVKKGDSTIMKSELEYGNCNTKCQTPMGAINSSMPFHNIHPLTIGECP
KYVKSNRLVLATGLRNSPQRERRRKKRGLFGAIAGFIEGGWQGMVDGWYG
YHHSNEQGSGYAADKESTQKAIDGVTNKVNSIIDKMNTQFEAVGREFNNLER
RIENLNKKMEDGFLDVWTYNAELLVLMENERTLDFHDSNVKNLYDKVRLQL
RDNAKELGNGCFEFYHKCDNECMESVRNGTYDYPQYSEEARLKREEISGVKL
ESIGIYQILSIYSTVASSLALAIMVAGLSLWMCSNGSLQCRICI
Antigen: H7N9 A/Anhui/1/2013 HA1, Virus: Influenza, Strain: A/Anhui/1/2013,
Uniprot/Genbank: AGJ51953.1, Expression: HEK293, SinoCatalog: 40103-V08H1
(SEQ ID NO: 14)
MNTQILVFALIAIIPTNADKICLGHHAVSNGTKVNTLTERGVEVVNATETVER
TNIPRICSKGKRTVDLGQCGLLGTITGPPQCDQFLEFSADLIIERREGSDVCYPG
KFVNEEALRQILRESGGIDKEAMGFTYSGIRTNGATSACRRSGSSFYAEMKW
LLSNTDNAAFPQMTKSYKNTRKSPALIVWGIHHSVSTAEQTKLYGSGNKLVT
VGSSNYQQSFVPSPGARPQVNGLSGRIDFHWLMLNPNDTVTFSFNGAFIAPDR
ASFLRGKSMGIQSGVQVDANCEGDCYHSGGTIISNLPFQNIDSRAVGKCPRYV
KQRSLLLATGMKNVPEIPKGRGLFGAIAGFIENGWEGLIDGWYGFRHQNAQG
EGTAADYKSTQSAIDQITGKLNRLIEKTNQQFELIDNEFNEVEKQIGNVINWTR
DSITEVWSYNAELLVAMENQHTIDLADSEMDKLYERVKRQLRENAEEDGTG
CFEIFHKCDDDCMASIRNNTYDHSKYREEAMQNRIQIDPVKLSSGYKDVILWF
SFGASCFILLAIVMGLVFICVKNGNMRCTICI
Antigen: H7N9 A/Anhui/1/2013 HA1 + HA2, Virus: Influenza,
Strain: A/Anhui/1/2013, Uniprot/Genbank: AGJ51953.1, Expression: HEK293,
SinoCatalog: 40103-V08H
(SEQ ID NO: 15)
MNTQILVFALIAIIPTNADKICLGHHAVSNGTKVNTLTERGVEVVNATETVER
TNIPRICSKGKRTVDLGQCGLLGTITGPPQCDQFLEFSADLIIERREGSDVCYPG
KFVNEEALRQILRESGGIDKEAMGFTYSGIRTNGATSACRRSGSSFYAEMKW
LLSNTDNAAFPQMTKSYKNTRKSPALIVWGIHHSVSTAEQTKLYGSGNKLVT
VGSSNYQQSFVPSPGARPQVNGLSGRIDFHWLMLNPNDTVTFSFNGAFIAPDR
ASFLRGKSMGIQSGVQVDANCEGDCYHSGGTHISNLPFQNIDSRAVGKCPRYV
KQRSLLLATGMKNVPEIPKGRGLFGAIAGFIENGWEGLIDGWYGFRHQNAQG
EGTAADYKSTQSAIDQITGKLNRLIEKTNQQFELIDNEFNEVEKQIGNVINWTR
DSITEVWSYNAELLVAMENQHTIDLADSEMDKLYERVKRQLRENAEEDGTG
CFEIFHKCDDDCMASIRNNTYDHSKYREEAMQNRIQIDPVKLSSGYKDVILWF
SFGASCFILLAIVMGLVFICVKNGNMRCTICI
Antigen: hAdV-3/45659_Fiber, Virus: Adenovirus, Strain: hAdV-3/45659,
Uniprot/Genbank: P04501, Expression: E., coli, SinoCatalog: UN5-9
(SEQ ID NO: 16)
MAKRARLSTSFNPVYPYEDESSSQHPFINPGFISPDGFTQSPNGVLSLKCVNPL
TTASGSLQLKVGSGLTVDTTDGSLEENIKVNTPLTKSNHSINLPIGNGLQIEQN
KLCSKLGNGLTFDSSNSIALKNNTLWTGPKPEANCHIEYGKQNPDSKLTLILVK
NGGIVNGYVTLMGASDYVNTLFKNKNVSINVELYFDATGHILPDSSSLKTDLE
LKYKQTADFSARGFMPSTTAYPFVLPNAGTHNENYIFGQCYYKASDGALFPL
EVTVMLNKRLPDSRTSYVMTFLWSLNAGLAPETTQATLITSPFTFSYIREDD
Antigen: hAdV-3/45659_Penton, Virus: Adenovirus, Strain: hAdV-3/45659,
Uniprot/Genbank: Q2Y0H9, Expression: Insect, Cells, SinoCatalog: UN5-10
(SEQ ID NO: 17)
MRRRAVLGGAVVYPEGPPPSYESVMQQQAAMIQPPLEAPFVPPRYLAPTEGR
NSIRYSELSPLYDTTKLYLVDNKSADIASLNYQNDHSNFLTTVVQNNDFTPTE
ASTQTINFDERSRWGGQLKTIMHTNMPNVNEYMFSNKFKARVMVSRKAPEG
VTVNDTYDHKEDILKYEWFEFILPEGNFSATMTIDLMNNAIIDNYLEIGRQNG
VLESDIGVKFDTRNFRLGWDPETKLIMPGVYTYEAFHPDIVLLPGCGVDFTES
RLSNLLGIRKRHPFQEGFKIMYEDLEGGNIPALLDVTAYEESKKDTTTETTTLA
VAEETSEDDDITRGDTYITEKQKREAAAAEVKKELKIQPLEKDSKSRSYNVLE
DKINTAYRSWYLSYNYGNPEKGIRSWTLLTTSDVTCGAEQVYWSLPDMMQD
PVTFRSTRQVNNYPVVGAELMPVFSKSFYNEQAVYSQQLRQATSLTHVFNRF
PENQILIRPPAPTITTVSENVPALTDHGTLPLRSSIRGVQRVTVTDARRRTCPYV
YKALGIVAPRVLSSRTF
Antigen: hAdV-4/28280_Fiber, Virus: Adenovirus, Strain: hAdV-4/28280,
Uniprot/Genbank: P36844, Expression: Insect, Cells, SinoCatalog: UN5-14
(SEQ ID NO: 18)
MSKSARGWSDGFDPVYPYDADNDRPCPSSTLPSFSSDGFQEKPLGVLSLGPG
RPCHTKNGEITLKLGEGVDLDDSGKLIANTVNKAIAPLSFFQQHHFPLTWIPLY
TPKMENYPYKFLPPLSILKSTILNTLVSAFGSGLGLSGSALAVQLASPLTFDDK
GNIKITLNRGLHVTTGDAIESNISWAKGIKFEDGAIATNIGKGSRFGTSSTETGV
NNAYPIQVKLGSGLSFDSTGAIMAGNKDYDKLTLWTTPDPSPNCQILAENDA
KLTLCLTMCDSQILATVSVLVVRSGNLNPITGTVSSAQVFLRFDANGVLLTEH
STSKKYWGYKQGDSIDGTPYTNAVGFMPNSTAYPKTQSSTTKNNIVGQVYM
NGDVSKPMLLTITLNGTDDTTSAYSMSFSYTWTNGSYIGATFGANSYTFSYIA
QQ
Antigen: hAdV-4/28280_Penton, Virus: Adenovirus, Strain: hAdV-4/28280,
Uniprot/Genbank: Q2KSF3, Expression: Insect, Cells, SinoCatalog: UN5-15
(SEQ ID NO: 19)
MMRRAYPEGPPPSYESVMQQAMAAAAAIQPPLEAPYVPPRYLAPTEGRNSIR
YSELTPLYDTTRLYLVDNKSADIASLNYQNDHSNFLTTVVQNNDFTPTEASTQ
TINFDERSRWGGQLKTIMHTNMPNVNQFMYSNKFKARVMVSRKTPNGVTV
GDNYDGSQDELKYEWVEFELPEGNFSVTMTIDLMNNAIIDNYLAVGRQNGV
LESDIGVKFDTRNFRLGWDPVTELVMPGVYTNEAFHPDIVLLPGCGVDFTESR
LSNLLGIRKRQPFQEGFQIMYEDLDGGNIPALLDVEAYEKSKEESVAAATTAV
ATASTEVRDDNFASAAAVAAVKADETKSKIVIQPVEKDSKERSYNVLSDKKN
TAYRSWYLAYNYGDRDKGVRSWTLLTTSDVTCGVEQVYWSLPDMMQDPV
TFRSTHQVSNYPVVGAELLPVYSKSFFNEQAVYSQQLRAFTSLTHVFNRFPEN
QILVRPPAPTITTVSENVPALTDHGTLPLRSSIRGVQRVTVTDARRRTCPYVYK
ALGIVAPRVLSSRTF
Antigen: HCoV-229E_S1, Virus: CommonCoV, Strain: 229E, Uniprot/Genbank:
A0A1L7B942, Expression: HEK293, SinoCatalog: UN1-2
(SEQ ID NO: 20)
MFVLLVAYALLHIAGCQTTNGTNTSHSVCNGCVGHSENVFAVESGGYIPSNF
SFNNWFLLTNTSSVVDGVVRSFQPLLLNCLWSVSGSQFTTGFVYFNGTGRGA
CKGFYSNASSDVIRYNINFEENLRRGTILFKTSYGAVVFYCTNNTLVSGDAHIP
SGTVLGNFYCFVNTTIGNETTSAFVGALPKTVREFVISRTGHFYINGYRYFSLG
DVEAVNFNVTNAATIVCTVALASYADVLVNVSQTAIANIIYCNSVINRLRCDQ
LSFDVPDGFYSTSPIQPVELPMSIVSLPVYHKHTFIVLHVKFEHQRGPGKCYNC
RPSVINITLANFNETKGPLCVDTSHFTTQFVDNVKLARWSASINTGNCPFSFGK
VNNFVKFGSVCFSLKDIPGGCAMPIMANLVNHKSHNIGSLYVSWSDGDVITG
VPKPVEGVSSFMNVTLNKCTKYNIYDVSGVGVIRISNDTFLNGITYTSTSGNLL
GFKDVTNGTIYSITPCNPPDQLVVYQQAVVGAMLSENFTSYGFSNVVEMPKF
FYASNGTYNCTDAVLTYSSFGVCADGSIIAVQPRNVSYDSVSAIVTANLSIPSN
WTTSVQVEYLQITSKPIVVDCSTYVCNGNVRCVELLKQYTSACKTIEDALRNS
AMLESADVSEMLTFDKKAFTLANVSSFGDYNLSSVIPSLPRSGSRVAGRSAIE
DILFSKLVTSGLGTVDADYKKCTKGLSIADLACAQYYNGIMVLPGVADAERM
AMYTGSLIGGIALGGLTSAASIPFSLAIQSRLNYVALQTDVLQENQKILAASFN
KAMTNIVDAFTGVNDAITQTSQALQTVATALNKIQDVVNQQGNSLNHLTSQL
RQNFQAISSSIQAIYDRLDIIQADQQVDRLITGRLAALNVFVSHTLTKYTEVRA
SRQLAQQKVNECVKSQSKRYGFCGNGTHIFSLVNAAPEGLVFLHTVLLPTQY
KDVEAWSGLCVDGINGYVLRQPNLALYKEGNYYRITSRIMFEPRIPTIADFVQI
ENCNVTFVNISRSELQTIVPEYIDVNKTLQELSYKLPNYTVPDLVVEQYNQTIL
NLTSEISTLENKSAELNYTVQKLQTLIDNINSTLVDLKWLNRVETYIKWPWW
VWLCISVVLIFVVSMLLLCCCSTGCCGFFSCFASSIKGCCESTKLPYYDVEKIHI
Q
Antigen: HCoV-229E_S1 + S2, Virus: CommonCoV, Strain: 229E, Uniprot/Genbank:
A0A1L7B942, Expression: Insect, Cells, SinoCatalog: UN2-2
(SEQ ID NO: 21)
MFVLLVAYALLHIAGCQTTNGTNTSHSVCNGCVGHSENVFAVESGGYIPSNF
SFNNWFLLTNTSSVVDGVVRSFQPLLLNCLWSVSGSQFTTGFVYFNGTGRGA
CKGFYSNASSDVIRYNINFEENLRRGTILFKTSYGAVVFYCTNNTLVSGDAHIP
SGTVLGNFYCFVNTTIGNETTSAFVGALPKTVREFVISRTGHFYINGYRYFSLG
DVEAVNFNVTNAATIVCTVALASYADVLVNVSQTAIANIIYCNSVINRLRCDQ
LSFDVPDGFYSTSPIQPVELPMSIVSLPVYHKHTFIVLHVKFEHQRGPGKCYNC
RPSVINITLANFNETKGPLCVDTSHFTTQFVDNVKLARWSASINTGNCPFSFGK
VNNFVKFGSVCFSLKDIPGGCAMPIMANLVNHKSHNIGSLYVSWSDGDVITG
VPKPVEGVSSFMNVTLNKCTKYNIYDVSGVGVIRISNDTFLNGITYTSTSGNLL
GFKDVTNGTIYSITPCNPPDQLVVYQQAVVGAMLSENFTSYGFSNVVEMPKF
FYASNGTYNCTDAVLTYSSFGVCADGSIIAVQPRNVSYDSVSAIVTANLSIPSN
WTTSVQVEYLQITSKPIVVDCSTYVCNGNVRCVELLKQYTSACKTIEDALRNS
AMLESADVSEMLTFDKKAFTLANVSSFGDYNLSSVIPSLPRSGSRVAGRSAIE
DILFSKLVTSGLGTVDADYKKCTKGLSIADLACAQYYNGIMVLPGVADAERM
AMYTGSLIGGIALGGLTSAASIPFSLAIQSRLNYVALQTDVLQENQKILAASFN
KAMTNIVDAFTGVNDAITQTSQALQTVATALNKIQDVVNQQGNSLNHLTSQL
RQNFQAISSSIQAIYDRLDIIQADQQVDRLITGRLAALNVFVSHTLTKYTEVRA
SRQLAQQKVNECVKSQSKRYGFCGNGTHIFSLVNAAPEGLVFLHTVLLPTQY
KDVEAWSGLCVDGINGYVLRQPNLALYKEGNYYRITSRIMFEPRIPTIADFVQI
ENCNVTFVNISRSELQTIVPEYIDVNKTLQELSYKLPNYTVPDLVVEQYNQTIL
NLTSEISTLENKSAELNYTVQKLQTLIDNINSTLVDLKWLNRVETYIKWPWW
VWLCISVVLIFVVSMLLLCCCSTGCCGFFSCFASSIKGCCESTKLPYYDVEKIHI
Q
Antigen: HCoV-HKU1_HE, Virus: CommonCoV, Strain: HKU1, Uniprot/Genbank:
Q0ZME7, Expression: HEK293, SinoCatalog: UN1-4-1
(SEQ ID NO: 22)
MFLIIFILPTTLAVIGDFNCTNSFINDYNKTIPRISEDVVDVSLGLGTYYVLNRV
YLNTTLLFTGYFPKSGANFRDLALKGSIYLSTLWYKPPFLSDFNNGIFSKVKN
TKLYVNNTLYSEFSTIVIGSVFVNTSYTIVVQPHNGILEITACQYTMCEYPHTV
CKSKGSIRNESWHIDSSEPLCLFKKNFTYNVSADWLYFHFYQERGVFYAYYA
DVGMPTTFLFSLYLGTILSHYYVMPLTCNAISSNTDNETLEYWVTPLSRRQYL
LNFDEHGVITNAVDCSSSFLSEIQCKTQSFAPNTGVYDLSGFTVKPVATVYRRI
PNLPDCDIDNWLNNVSVPSPLNWERRIFSNCNFNLSTLLRLVHVDSFSCNNLD
KSKIFGSCFNSITVDKFAIPNRRRDDLQLGSSGFLQSSNYKIDISSSSCQLYYSLP
LVNVTINNFNPSSWNRRYGFGSFNLSSYDVVYSDHCFSVNSDFCPCADPSVV
NSCAKSKPPSAICPAGTKYRHCDLDTTLYVKNWCRCSCLPDPISTYSPNTCPQ
KKVVVGIGEHCPGLGINEEKCGTQLNHSSCFCSPDAFLGWSFDSCISNNRCNIF
SNFIFNGINSGTTCSNDLLYSNTEISTGVCVNYDLYGITGQGIFKEVSAAYYNN
WQNLLYDSNGNIIGFKDFLTNKTYTILPCYSGRVSAAFYQNSSSPALLYRNLK
CSYVLNNISFISQPFYFDSYLGCVLNAVNLTSYSVSSCDLRMGSGFCIDYALPS
SRRKRRGISSPYRFVTFEPFNVSFVNDSVETVGGLFEIQIPTNFTIAGHEEFIQTS
SPKVTIDCSAFVCSNYAACHDLLSEYGTFCDNINSILNEVNDLLDITQLQVAN
ALMQGVTLSSNLNTNLHSDVDNIDFKSLLGCLGSQCGSSSRSLLEDLLFNKVK
LSDVGFVEAYNNCTGGSEIRDLLCVQSFNGIKVLPPILSETQISGYTTAATVAA
MFPPWSAAAGVPFSLNVQYRINGLGVTMDVLNKNQKLIANAFNKALLSIQNG
FTATNSALAKIQSVVNANAQALNSLLQQLFNKFGAISSSLQEILSRLDNLEAQ
VQIDRLINGRLTALNAYVSQQLSDITLIKAGASRAIEKVNECVKSQSPRINFCG
NGNHILSLVQNAPYGLLFIHFSYKPTSFKTVLVSPGLCLSGDRGIAPKQGYFIK
QNDSWMFTGSSYYYPEPISDKNVVFMNSCSVNFTKAPFIYLNNSIPNLSDFEA
ELSLWFKNHTSIAPNLTFNSHINATFLDLYYEMNVIQESIKSLNSSFINLKEIGT
YEMYVKWPWYIWLLIVILFIIFLMILFFICCCTGCGSACFSKCHNCCDEYGGHN
DFVIKASHDD
Antigen: HCoV-HKU1_S1,N-(AA1-760), Virus: CommonCoV, Strain: HKU1,
Uniprot/Genbank: YP_173238.1, Expression: HEK293, SinoCatalog: 40021-V08H
(SEQ ID NO: 23)
GDFNCTNFAINDLNTTVPRISEYVVDVSYGLGTYYILDRVYLNTTILFTGYFPK
SGANFRDLSLKGTTYLSTLWYQKPFLSDFNNGIFSRVKNTKLYVNKTLYSEFS
TIVIGSVFINNSYTIVVQPHNGVLEITACQYTMCEYPHTICKSKGSSRNESWHF
DKSEPLCLFKKNFTYNVSTDWLYFHFYQERGTFYAYYADSGMPTTFLFSLYL
GTLLSHYYVLPLTCNAISSNTDNETLQYWVTPLSKRQYLLKFDNRGVITNAV
DCSSSFFSEIQCKTKSLLPNTGVYDLSGFTVKPVATVHRRIPDLPDCDIDKWLN
NFNVPSPLNWERKIFSNCNFNLSTLLRLVHTDSFSCNNFDESKIYGSCFKSIVL
DKFAIPNSRRSDLQLGSSGFLQSSNYKIDTTSSSCQLYYSLPAINVTINNYNPSS
WNRRYGFNNFNLSSHSVVYSRYCFSVNNTFCPCAKPSFASSCKSHKPPSASCP
IGTNYRSCESTTVLDHTDWCRCSCLPDPITAYDPRSCSQKKSLVGVGEHCAGF
GVDEEKCGVLDGSYNVSCLCSTDAFLGWSYDTCVSNNRCNIFSNFILNGINSG
TTCSNDLLQPNTEVFTDVCVDYDLYGITGQGIFKEVSAVYYNSWQNLLYDSN
GNIIGFKDFVTNKTYNIFPCYAGRVSAAFHQNASSLALLYRNLKCSYVLNNIS
LTTQPYFDSYLGCVFNADNLTDYSVSSCALRMGSGFCVDYNSPSSSSSRRKRR
AHHHHHHHHHH
Antigen: HCoV-HKU1_S1,N-(AA13-756), Virus: CommonCoV, Strain: HKU1,
Uniprot/Genbank: Q0ZME7, Expression: HEK293, SinoCatalog: UN1-3
(SEQ ID NO: 24)
MFLIIFILPTTLAVIGDFNCTNSFINDYNKTIPRISEDVVDVSLGLGTYYVLNRV
YLNTTLLFTGYFPKSGANFRDLALKGSIYLSTLWYKPPFLSDFNNGIFSKVKN
TKLYVNNTLYSEFSTIVIGSVFVNTSYTIVVQPHNGILEITACQYTMCEYPHTV
CKSKGSIRNESWHIDSSEPLCLFKKNFTYNVSADWLYFHFYQERGVFYAYYA
DVGMPTTFLFSLYLGTILSHYYVMPLTCNAISSNTDNETLEYWVTPLSRRQYL
LNFDEHGVITNAVDCSSSFLSEIQCKTQSFAPNTGVYDLSGFTVKPVATVYRRI
PNLPDCDIDNWLNNVSVPSPLNWERRIFSNCNFNLSTLLRLVHVDSFSCNNLD
KSKIFGSCFNSITVDKFAIPNRRRDDLQLGSSGFLQSSNYKIDISSSSCQLYYSLP
LVNVTINNFNPSSWNRRYGFGSFNLSSYDVVYSDHCFSVNSDFCPCADPSVV
NSCAKSKPPSAICPAGTKYRHCDLDTTLYVKNWCRCSCLPDPISTYSPNTCPQ
KKVVVGIGEHCPGLGINEEKCGTQLNHSSCFCSPDAFLGWSFDSCISNNRCNIF
SNFIFNGINSGTTCSNDLLYSNTEISTGVCVNYDLYGITGQGIFKEVSAAYYNN
WQNLLYDSNGNIIGFKDFLTNKTYTILPCYSGRVSAAFYQNSSSPALLYRNLK
CSYVLNNISFISQPFYFDSYLGCVLNAVNLTSYSVSSCDLRMGSGFCIDYALPS
SRRKRRGISSPYRFVTFEPFNVSFVNDSVETVGGLFEIQIPTNFTIAGHEEFIQTS
SPKVTIDCSAFVCSNYAACHDLLSEYGTFCDNINSILNEVNDLLDITQLQVAN
ALMQGVTLSSNLNTNLHSDVDNIDFKSLLGCLGSQCGSSSRSLLEDLLFNKVK
LSDVGFVEAYNNCTGGSEIRDLLCVQSFNGIKVLPPILSETQISGYTTAATVAA
MFPPWSAAAGVPFSLNVQYRINGLGVTMDVLNKNQKLIANAFNKALLSIQNG
FTATNSALAKIQSVVNANAQALNSLLQQLFNKFGAISSSLQEILSRLDNLEAQ
VQIDRLINGRLTALNAYVSQQLSDITLIKAGASRAIEKVNECVKSQSPRINFCG
NGNHILSLVQNAPYGLLFIHFSYKPTSFKTVLVSPGLCLSGDRGIAPKQGYFIK
QNDSWMFTGSSYYYPEPISDKNVVFMNSCSVNFTKAPFIYLNNSIPNLSDFEA
ELSLWFKNHTSIAPNLTFNSHINATFLDLYYEMNVIQESIKSLNSSFINLKEIGT
YEMYVKWPWYIWLLIVILFIIFLMILFFICCCTGCGSACFSKCHNCCDEYGGHN
DFVIKASHDD
Antigen: HCoV-HKU1_S1 + S2, Virus: CommonCoV, Strain: HKU1,
Uniprot/Genbank: Q0ZME7, Expression: Insect, Cells, SinoCatalog: UN2-3
(SEQ ID NO: 25)
MFLIIFILPTTLAVIGDFNCTNSFINDYNKTIPRISEDVVDVSLGLGTYYVLNRV
YLNTTLLFTGYFPKSGANFRDLALKGSIYLSTLWYKPPFLSDFNNGIFSKVKN
TKLYVNNTLYSEFSTIVIGSVFVNTSYTIVVQPHNGILEITACQYTMCEYPHTV
CKSKGSIRNESWHIDSSEPLCLFKKNFTYNVSADWLYFHFYQERGVFYAYYA
DVGMPTTFLFSLYLGTILSHYYVMPLTCNAISSNTDNETLEYWVTPLSRRQYL
LNFDEHGVITNAVDCSSSFLSEIQCKTQSFAPNTGVYDLSGFTVKPVATVYRRI
PNLPDCDIDNWLNNVSVPSPLNWERRIFSNCNFNLSTLLRLVHVDSFSCNNLD
KSKIFGSCFNSITVDKFAIPNRRRDDLQLGSSGFLQSSNYKIDISSSSCQLYYSLP
LVNVTINNFNPSSWNRRYGFGSFNLSSYDVVYSDHCFSVNSDFCPCADPSVV
NSCAKSKPPSAICPAGTKYRHCDLDTTLYVKNWCRCSCLPDPISTYSPNTCPQ
KKVVVGIGEHCPGLGINEEKCGTQLNHSSCFCSPDAFLGWSFDSCISNNRCNIF
SNFIFNGINSGTTCSNDLLYSNTEISTGVCVNYDLYGITGQGIFKEVSAAYYNN
WQNLLYDSNGNIIGFKDFLINKTYTILPCYSGRVSAAFYQNSSSPALLYRNLK
CSYVLNNISFISQPFYFDSYLGCVLNAVNLTSYSVSSCDLRMGSGFCIDYALPS
SRRKRRGISSPYRFVTFEPFNVSFVNDSVETVGGLFEIQIPTNFTIAGHEEFIQTS
SPKVTIDCSAFVCSNYAACHDLLSEYGTFCDNINSILNEVNDLLDITQLQVAN
ALMQGVTLSSNLNTNLHSDVDNIDFKSLLGCLGSQCGSSSRSLLEDLLFNKVK
LSDVGFVEAYNNCTGGSEIRDLLCVQSFNGIKVLPPILSETQISGYTTAATVAA
MFPPWSAAAGVPFSLNVQYRINGLGVTMDVLNKNQKLIANAFNKALLSIQNG
FTATNSALAKIQSVVNANAQALNSLLQQLFNKFGAISSSLQEILSRLDNLEAQ
VQIDRLINGRLTALNAYVSQQLSDITLIKAGASRAIEKVNECVKSQSPRINFCG
NGNHILSLVQNAPYGLLFIHFSYKPTSFKTVLVSPGLCLSGDRGIAPKQGYFIK
QNDSWMFTGSSYYYPEPISDKNVVFMNSCSVNFTKAPFIYLNNSIPNLSDFEA
ELSLWFKNHTSIAPNLTFNSHINATFLDLYYEMNVIQESIKSLNSSFINLKEIGT
YEMYVKWPWYIWLLIVILFIIFLMILFFICCCTGCGSACFSKCHNCCDEYGGHN
DFVIKASHDD
Antigen: HCoV-NL63_S1, Virus: CommonCoV, Strain: NL63, Uniprot/Genbank:
A0A1L2YVI8, Expression: HEK293, SinoCatalog: UN1-1
(SEQ ID NO: 26)
MKLFLILLVLPLASCFFTCNSNANLSMLQLGVPDNSSTIVTGLLPTHWICANQS
TSVYSANGFFYIDVGNHRSAFALHTGYYDVNQYYIYVTNEIGLNASVTLKICK
FGINTTFDFLSNSSSSFDCIVNLLFTEQLGAPLGITISGETVRLHLYNVTRTFYV
PAAYKLTKLSVKCYFNYSCVFSVVNATVTVNVTTHNGRVVNYTVCDDCNG
YTDNIFSVQQDGRIPNGFPFNNWFLLTNGSTLVDGVSRLYQPLRLTCLWPVPG
LKSSTGFVYFNATGSDVNCNGYQHNSVADVMRYNLNFSANSVDNLKSGVIV
FKTLQYDVLFYCSNSSSGVLDTTIPFGPSSQPYYCFINSTINTTHVSTFVGVLPP
TVREIVVARTGQFYINGFKYFDLGFIEAVNFNVTTASATDFWTVAFATFVDVL
VNVSATKIQNLLYCDSPFEKLQCEHLQFGLQDGFYSANFLDDNVLPETYVAL
PIYYQHTDINFTATASFGGSCYVCKPHQVNISLNGNTSVCVRTSHFSIR YIYNR
VKSGSPGDSSWHIYLKSGTCPFSFSKLNNFQKFKTICFSTVAVPGSCNFPLEAT
WHYTSYTIVGALYVTWSEGNSITGVPYPVSGIREFSNLVLNNCTKYNIYDYVG
TGIIRSSNQSLAGGITYVSNSGNLLGFKNVSTGNIFIVTPCNQPDQVAVYQQSII
GAMTAVNESRYGLQNLLQLPNFYYVSNGGNNCTTAVMTYSNFGICADGSLIP
VRLRNSSDNGISAIITANLSIPSNWTTSVQVEYLQITSIPIVVDCATYVCNGNPR
CKNLLKQYTSACKTIEDALRLSAHLETNDVSSMLTFDSNAFSLANVTSFGDY
NLSSVLPQRNIHSSRIAGRSALEDLLFSKVVTSGLGTVDVDYKSCTKGLSIADL
ACAQYYNGIMVLPGVADAERMAMYTGSLIGGMVLGGLTSAAAIPFSLALQA
RLNYVALQTDVLQENQKILAASFNKAINNIVASFSSVNDAITQTAEAIHTVTIA
LNKIQDVVNQQGSALNHLTSQLRHNFQAISNSIQAIYDRLDSIQADQQVDRLIT
GRLAALNAFVSQVLNKYTEVRSSRRLAQQKINECVKSQSNRYGFCGNGTHIF
SIVNSAPDGLLFLHTVLLPTDYKNVKAWSGICVDGIYGYVLRQPNLVLYSDN
GVFRVTSRVMFQPRLPVLSDFVQIYNCNVTFVNISRVELHTVIPDYVDVNKTL
QEFAQNLPKYVKPNFDLTPFNLTYLNLSSELKQLEAKTASLFQTTVELQGLID
QINSTYVDLKLLNRFENYIKWPWWVWLIISVVFVVLLSLLVFCCLSTGCCGCC
NCLTSSMRGCCDCGSTKLPYYEFEKVHVQ
>Antigen: HCoV-NL63_S1 + S2, Virus: CommonCoV, Strain: NL63,
Uniprot/Genbank: A0A1L2YVI8, Expression: Insect, Cells, SinoCatalog: UN2-1
(SEQ ID NO: 27)
MKLFLILLVLPLASCFFTCNSNANLSMLQLGVPDNSSTIVTGLLPTHWICANQS
TSVYSANGFFYIDVGNHRSAFALHTGYYDVNQYYIYVTNEIGLNASVTLKICK
FGINTTFDFLSNSSSSFDCIVNLLFTEQLGAPLGITISGETVRLHLYNVTRTFYV
PAAYKLTKLSVKCYFNYSCVFSVVNATVTVNVTTHNGRVVNYTVCDDCNG
YTDNIFSVQQDGRIPNGFPFNNWFLLTNGSTLVDGVSRLYQPLRLTCLWPVPG
LKSSTGFVYFNATGSDVNCNGYQHNSVADVMRYNLNFSANSVDNLKSGVIV
FKTLQYDVLFYCSNSSSGVLDTTIPFGPSSQPYYCFINSTINTTHVSTFVGVLPP
TVREIVVARTGQFYINGFKYFDLGFIEAVNFNVTTASATDFWTVAFATFVDVL
VNVSATKIQNLLYCDSPFEKLQCEHLQFGLQDGFYSANFLDDNVLPETYVAL
PIYYQHTDINFTATASFGGSCYVCKPHQVNISLNGNTSVCVRTSHFSIRYIYNR
VKSGSPGDSSWHIYLKSGTCPFSFSKLNNFQKFKTICFSTVAVPGSCNFPLEAT
WHYTSYTIVGALYVTWSEGNSITGVPYPVSGIREFSNLVLNNCTKYNIYDYVG
TGIIRSSNQSLAGGITYVSNSGNLLGFKNVSTGNIFIVTPCNQPDQVAVYQQSII
GAMTAVNESRYGLQNLLQLPNFYYVSNGGNNCTTAVMTYSNFGICADGSLIP
VRLRNSSDNGISAIITANLSIPSNWTTSVQVEYLQITSIPIVVDCATYVCNGNPR
CKNLLKQYTSACKTIEDALRLSAHLETNDVSSMLTFDSNAFSLANVTSFGDY
NLSSVLPQRNIHSSRIAGRSALEDLLFSKVVTSGLGTVDVDYKSCTKGLSIADL
ACAQYYNGIMVLPGVADAERMAMYTGSLIGGMVLGGLTSAAAIPFSLALQA
RLNYVALQTDVLQENQKILAASFNKAINNIVASFSSVNDAITQTAEAIHTVTIA
LNKIQDVVNQQGSALNHLTSQLRHNFQAISNSIQAIYDRLDSIQADQQVDRLIT
GRLAALNAFVSQVLNKYTEVRSSRRLAQQKINECVKSQSNRYGFCGNGTHIF
SIVNSAPDGLLFLHTVLLPTDYKNVKAWSGICVDGIYGYVLRQPNLVLYSDN
GVFRVTSRVMFQPRLPVLSDFVQIYNCNVTFVNISRVELHTVIPDYVDVNKTL
QEFAQNLPKYVKPNFDLTPFNLTYLNLSSELKQLEAKTASLFQTTVELQGLID
QINSTYVDLKLLNRFENYIKWPWWVWLIISVVFVVLLSLLVFCCLSTGCCGCC
NCLTSSMRGCCDCGSTKLPYYEFEKVHVQ
Antigen: HCoV-OC43_HE, Virus: CommonCoV, Strain: OC43, Uniprot/Genbank:
ATN39879.2, Expression: HEK293, SinoCatalog: UN1-6
(SEQ ID NO: 28)
MFLLPRFILVSCVIGSLGFYNPPTNVVSHVNGDWFLFGDSRSDCNHIVNINPH
NYSYMDLNPALCDSGKISSKAGNSIFRSFHFTDFYNYTGEGQQIIFYEGVNFTP
YHAFKCNRSGSNDIWMQNKGLFYTQVYKNMAVYRSLTFVNVSYVYNGSAQ
ATSFCKSGSKLKNLVLNNPAYIAPQAKSGIITKVEADFYLSGCDEYIVPLCIFN
GKSLSNTKYYDHSQYYFNKDTGVIYGLNSTETITTSFDLNCHYLVLPSGNYLA
ISNELLLTVPTKAICFNKRKDFTPVQVVDSRWNNARQSDNMTAVACQPPYCY
FRNSTTNYVGVYDINHGDAGFTSILSGLLYNSSCFSQQGVFRYDNISSVWPLY
PYGRCPTAAYINIPDLPICVYDPLPVILLGILLGVAIVIIVVLLLHFMVDNVTRL
HDA
Antigen: HCoV-OC43_S1, Virus: CommonCoV, Strain: OC43, Uniprot/Genbank:
AVR40344.1, Expression: HEK293, SinoCatalog: UN1-5
(SEQ ID NO: 29)
MFLILLISLPTAFAVIGDLNCTLDPRLKGSFNNRDTGPPSISIDTVDVTNGLGTY
YVLDRVYLNTTLFLNGYYPTSGSTYRNMALKGTDLLSTLWFKPPFLSDFINGI
FAKVKNTKVFKDGVMYSEFPAITIGSTFVNTSYSVVVQPRTINSTQDGVNKLQ
GLLEVSVCQYNMCEYPHTICHPNLGNHFKELWHYDTGVVSCLYKRNFTYDV
NATYLYFHFYQEGGTFYAYFTDTGFVTKFLFNVYLGMALSHYYVMPLTCIRR
PKDGFSLEYWVTPLTPRQYLLAFNQDGIIFNAVDCMSDFMSEIKCKTQSIAPPT
GVYELNGYTVQPVADVYRRKPDLPNCNIEAWLNDKSVPSPLNWERKTFSNC
NFNMSSLMSFIQADSFTCNNIDAAKIYGMCFSSITIDKFAIPNRRKVDLQLGNL
GYLQSSNYRIDTTATSCQLYYNLPAANVSVSRFNPSTWNKRFGFIEDSVFVPQ
PTGVFTNHSVVYAQHCFKAPKNFCPCSSCSCPGKNNGIGTCPAGTNSLTCDNL
CTLDPITLKAPDTYKCPQSKSLVGIGEHCSGLAVKSDYCGNNSCTCQPQAFLG
WSADSCLQGDKCNIFANFILHDVNNGLTCSTDLQKANTEIELGVCVNYDLYG
ISGQGIFVEVNATYYNSWQNLLYDSNGNLYGFRDYITNRTFMIHSCYSGRVS
AAYHANSSEPALLFRNIKCNYVFNNSLTRQLQPINYSFDSYLGCVVNAYNSTA
ISVQTCDLTVGSGYCVDYSKNRRSRRAITTGYRFTNFEPFTVNSVNDSLEPVG
GLYEIQIPSEFTIGNMEEFIQTSSPKVTIDCAAFVCGDYAACKLQLVEYGSFCD
NINAILTEVNELLDTTQLQVANSLMNGVTLSTKLKDGVNFNVDDINFSPVLGC
LGSECSKASSRSAIEDLLFDKVKLSDVGFVEAYNNCTGGAEIRDLICVQSYKGI
KVLPPLLSENQISGYTLAATSASLFPPWTAAAGVPFYLNVQYRINGLGVTMD
VLSQNQKLIANAFNNALHAIQQGFDATNSALVKIQAVVNANAEALNNLLQQL
SNRFGAISASLQEILSRLDALEAEAQIDRLINGRLTALNAYVSQQLSDSTLVKF
SAAQAMEKVNECVKSQSSRINFCGNGNHIISLVQNAPYGLYFIHFNYVPTKYV
TAKVSPGLCIAGNRGIAPKSGYFVNVNNTWMYTGSGYYYPEPITENNVVVMS
TCAVNYTKAPYVMLNTSIPNLPDFKEELDQWFKNQTSVAPDLSLDYINVTFL
DLQVEMNRLQEAIKVLNHSYINLKDIGTYEYYVKWPWYVWLLICLAGVAML
VLLFFICCCTGCGTSCFKKCGGCCDDYTGYQELVIKTSHDD
Antigen: HCoV-OC43_S1 + S2, Virus: CommonCoV, Strain: OC43, Uniprot/Genbank:
AVR40344.1, Expression: Insect, Cells, SinoCatalog: UN2-4
(SEQ ID NO: 30)
MFLILLISLPTAFAVIGDLNCTLDPRLKGSFNNRDTGPPSISIDTVDVTNGLGTY
YVLDRVYLNTTLFLNGYYPTSGSTYRNMALKGTDLLSTLWFKPPFLSDFINGI
FAKVKNTKVFKDGVMYSEFPAITIGSTFVNTSYSVVVQPRTINSTQDGVNKLQ
GLLEVSVCQYNMCEYPHTICHPNLGNHFKELWHYDTGVVSCLYKRNFTYDV
NATYLYFHFYQEGGTFYAYFTDTGFVTKFLFNVYLGMALSHYYVMPLTCIRR
PKDGFSLEYWVTPLTPRQYLLAFNQDGIIFNAVDCMSDFMSEIKCKTQSIAPPT
GVYELNGYTVQPVADVYRRKPDLPNCNIEAWLNDKSVPSPLNWERKTFSNC
NFNMSSLMSFIQADSFTCNNIDAAKIYGMCFSSITIDKFAIPNRRKVDLQLGNL
GYLQSSNYRIDTTATSCQLYYNLPAANVSVSRFNPSTWNKRFGFIEDSVFVPQ
PTGVFTNHSVVYAQHCFKAPKNFCPCSSCSCPGKNNGIGTCPAGTNSLTCDNL
CTLDPITLKAPDTYKCPQSKSLVGIGEHCSGLAVKSDYCGNNSCTCQPQAFLG
WSADSCLQGDKCNIFANFILHDVNNGLTCSTDLQKANTEIELGVCVNYDLYG
ISGQGIFVEVNATYYNSWQNLLYDSNGNLYGFRDYITNRTFMIHSCYSGRVS
AAYHANSSEPALLFRNIKCNYVFNNSLTRQLQPINYSFDSYLGCVVNAYNSTA
ISVQTCDLTVGSGYCVDYSKNRRSRRAITTGYRFTNFEPFTVNSVNDSLEPVG
GLYEIQIPSEFTIGNMEEFIQTSSPKVTIDCAAFVCGDYAACKLQLVEYGSFCD
NINAILTEVNELLDTTQLQVANSLMNGVTLSTKLKDGVNFNVDDINFSPVLGC
LGSECSKASSRSAIEDLLFDKVKLSDVGFVEAYNNCTGGAEIRDLICVQSYKGI
KVLPPLLSENQISGYTLAATSASLFPPWTAAAGVPFYLNVQYRINGLGVTMD
VLSQNQKLIANAFNNALHAIQQGFDATNSALVKIQAVVNANAEALNNLLQQL
SNRFGAISASLQEILSRLDALEAEAQIDRLINGRLTALNAYVSQQLSDSTLVKF
SAAQAMEKVNECVKSQSSRINFCGNGNHIISLVQNAPYGLYFIHFNYVPTKYV
TAKVSPGLCIAGNRGIAPKSGYFVNVNNTWMYTGSGYYYPEPITENNVVVMS
TCAVNYTKAPYVMLNTSIPNLPDFKEELDQWFKNQTSVAPDLSLDYINVTFL
DLQVEMNRLQEAIKVLNHSYINLKDIGTYEYYVKWPWYVWLLICLAGVAML
VLLFFICCCTGCGTSCFKKCGGCCDDYTGYQELVIKTSHDD
Antigen: hMPV-A_G(52N-228N), Virus: hMPV, Strain: PER/CFI0320/2010/A,
Glycoprotein, Uniprot/Genbank: X4XYT2, Expression: HEK293, SinoCatalog: UN6-3
(SEQ ID NO: 31)
MEVKVENIRAIDMLKARVKNRVARSKCFKNASLILIGITTLSIALNIYLIINYTI
QKTTSESEHHTSSPPTESNKETSTIPTDNPDITPNSQHPTQQSTESPTLHPASSTS
PPETEPASTPGTTNRPSFVDRSTTQSSESRTKTNSTVHKRNISSTTPRTQFPPRT
TTKAVSRTTAFRTSSTGERPTTTSVQSDSSTTARNHEETGSANPQASASTMQT
QHTNIAKPN
Antigen: hMPV-B_F(280D-490G), Virus: hMPV, Strain: PER/CFI0320/2010/A,
Glycoprotein, Uniprot/Genbank: F4YZY2, Expression: HEK293, SinoCatalog: UN6-5
(SEQ ID NO: 32)
MSWKVMIIISLLITPQHGLKESYLEESCSTITEGYLSVLRTGWYTNVFTLEVGD
VENLTCTDGPSLIKTELDLTKSALRELKTVSADQLAREEQIENPRQSRFVLGAI
ALGVATAAAVTAGIAIAKTIRLESEVNAIKGALKTTNEAVSTLGNGVRVLATA
VRELKEFVSKNLTSAINKNKCDIADLKMAVSFSQFNRRFLNVVRQFSDNAGIT
PAISLDLMNDAELARAVSYMPTSAGQIKLMLENRAMVRRKGFGILIGVYGSS
VIYMVOLPIFGVINTPCWIIKAAPSCSEKDGNYACLLREDQGWYCKNAGSTV
YYPNEKDCETRGDHVFCDTAAGINVAEQSRECNINISTTNYPCKVSTGRHPIS
MVALSPLGALVACYKGVSCSTGSNQVGIIKQLPKGCSYITNQDADTVTIDNTV
YQLSKVEGEQHVIKGRPVSSSFDPIRFPEDQFNVALDQVFESIENSQALVDQSN
KILNSAEKGNTGFIIVIILIAVLGLTMISVSIIIIIKKTRKPTGAPPELNGVTNGGFI
PHS
Antigen: hMPV-B_G(52D-238S), Virus: hMPV, Strain: PER/CFI0466/2010/B,
Glycoprotein, Uniprot/Genbank: X4Y8Q3, Expression: HEK293, SinoCatalog: UN6-6
(SEQ ID NO: 33)
MEVRVENIRAIDMFKAKMKNRIRSSKCYRNATLILIGLTALSMALNIFLIIDYA
TLKNMTKVEHCVNMPPVEPSKKTPMTSAVDLNTKLNPQQATQLTTEDSTSLA
ATSENHLHTGTTPTSDATISQQATDEHTTLLRSINRQTTQTTTEKKPTGAITKK
EKETTTRTTSTAATQTLNTTNQISNGREATTTSARSRNGATTQNSDQTIQAAD
PSSKPYHTQKSTTTAHNADTSSLSS
Antigen: hPIV-11203_F, Virus: Parainfluenza, Strain: hPIV-1, 12O3,
Uniprot/Genbank: A0A1V0E1X5, Expression: Insect, Cells, SinoCatalog: UN5-1
(SEQ ID NO: 34)
MQSSEVLLLVYSSLLLSSSLCQIPVDKLSNVGVIINEGKLLKIAGSYESRYIVLS
LVPSIDLQDGCGTTQIIQYKNLLNRLLIPLKDALDLQESLITITNDTTVTNDNPQ
TRFFGAVIGTIALGVATAAQITAGIALAEAREARKDIALIKDSIVKTHNSVEFIQ
RGIGEQIIALKTLQDFVNDEIRPAIGELRCETTALKLGIKLTQHYSELATAFSSN
LGTIGEKSLTVQALSSLYSANITEILSTIKKDKSDIYDIIYTEQVKGTVIDVDLEK
YMVTLLVKIPILSEIPGVLIYRASSISYNIEGEEWHVAIPNYIINKASSLGGADVT
NCIESKLAYICPRDPTQLIPDNQQKCILGDVSKCPVTKVINNLVPKFAFINGGV
VANCIASTCTCGTNRIPVNQDRSKGVTFLTYTNCGLIGINGIELYANKRGRDTT
WGNQIIKVGPAVSIRPVDISLNLASATNFLEESKTELMKARAIISAVGGWHNK
ESTQIIIIIIVCVLIIIICSILYYLYRVRRLLIMINSTNNSPINAYTLESRMKNPYMG
NHSN
Antigen: hPIV-11203_H, Virus: Parainfluenza, Strain: hPIV-1, 12O3,
Uniprot/Genbank: A0A1B2CW87, Expression: Insect, Cells, SinoCatalog: UN5-2
(SEQ ID NO: 35)
MAEKGKTINSYWSTTRNDNSTVNTHINTPAGRTHIWLLIATAMHATLSLIIMIL
CIDLIMKQDTCMKTNTMTVSSVNESAKTIKETITELIRQEVISRTINIQSSVQSGI
PILLNKQSRDLTQLIEKSCNKRELAQICENTIAIHHADGITPLDPHDFWRCPVG
EPLLSNNPNISLLPGPSLLSGSSTISGCVRLPSLSIGDAIYAYSSNLITQGCADIG
KSYQVLQLGYISLNSDMYPDLNPVISHTYDINDNRKSCSVIAAGTRGYQLCSL
PTVNETTDYSSEGIEDLVFDILDLKGKTKSHRYKNEDITFDHPFSAMYPSVGS
GIKIEDTLVFLGYGGLTTPLQGNTKCVISKCPNVNQSVCNDALKITWLKKRQV
VNVLIRINNYLSDRPKIVVETIPITQNYLGAEGRLLKLGKKIYIYTRSSGWHSN
LQIGSLDINKPMTINWTPHRVLSRPGNPDCNWFNKCPRECISGVYTDAYPLSP
DAVNVATTTLYANTSRVNPTIMYSNTSKIINMLRLKNGKLEAAYTTTSCITHF
GKGYCFHIVEINQTSLNTFQPMLFKTSIPKVCKITS
Antigen: hPIV-3USA/10991B/2010_H, Virus: Parainfluenza, Strain: hPIV-3,
USA/10991B/2010, Uniprot/Genbank: T1UD13, Expression: Insect, Cells,
SinoCatalog: UN5-6
(SEQ ID NO: 36)
MEYWKHTNHGKDAGNELETSTATHGNKLTNKITYILWTITLVLLSIVFIIVLT
NSIKSERARESLLQDINNEFMEVTEKIQVASDNTNDLIQSGVNTRLLTIQSHVQ
NYIPISLTQQISDLRKFISEITIRNDNQEVPPQRITHDVGIKPLNPDDFWRCTSGL
PSLMKTPKIRLMPGPGLLAMPTTVDGCVRTPSLVINDLIYAYTSNLITRGCQDI
GKSYQVLQIGIITVNSDLVPDLNPRISHTFNINDNRKSCSLALLNTDVYQLCST
PKVDERSDYASSGIEDIVLDIVNYDGSISTTRFKNNNISFDQPYAALYPSVGPGI
YYKGKIIFLGYGGLEHPINENAICNTTGCPGKTQRDCNQASHSPWFSDRRMV
NSIIVVDKGLNSVPKLKVWTISMRQNYWGSEGRLLLLGNKIYIYTRSTSWHSK
LQLGIIDITDYSDIRIKWTWHNVLSRPGNNECPWGHSCPDGCITGVYTDAYPL
NPTGSIVSSVILDSQKSRVNPVITYSTATERVNELAIRNKTLSAGYTTTSCITHY
NKGYCFHIVEINHKSLNTFQPMLFKTEIPKSCS
Antigen: hPIV-46/10-H2/2016_H, Virus: Parainfluenza, Strain: hPIV-4b/10-H2/2016,
Uniprot/Genbank: A0A1V0E1N4, Expression: Insect, Cells, SinoCatalog: UN5-8
(SEQ ID NO: 37)
MQDSRGNTQIFSQANSMVKRTWRLLFRIVTLILLISIFVLSLIIVLQSTPGNLQS
DVDIIRKELDELKENFETTSKSLLSVANQITYDVSVLTPIRQEATETNIIAKIKD
HCKDRVVKGESTCTLGHKPLHDVSFLNGFNKFYFTYRDNVQIRLNPLLDYPN
FIPTATTPHGCIRIPSFSLSQTHWCYTHNTILRGCEDTASSKQYVSLGTLQTLEN
GDPYFKVEYSHYLNDRKNRKSCSVVAVLDGCLLYCVIMTKNETENFKDPQL
ATQLLTYISYNGTIKERIINPPGSSRDWVHISPGVGSGILYSNYIIFPLYGGLME
NSMIYNNQSGKYFFPNSTKLPCSNKTSEKITGAKDSYTITYFSGRLIQSAFLICD
LRQFLSEDCEILIPSNDHMLVGAEGRLYNIENNIFYYQRGSSWWPYPSLYRIKL
NSNKKYPRIIETKFTKIEIAPRPGNKDCPGNKACPKECITGVYQDIWPLSYPNT
AFPHKKRAYYTGFYLNNSLARRNPTFYTADNLDYHQQERLGKFNLTAGYST
TTCFKQTTTARLYCLYILEVGDSVIGDFQIFSFLRSIDQAIT
Antigen: MERS-COV_NP, Virus: MERS, Strain: MERS, Uniprot/Genbank:
AFS88943.1, Expression: Insect, Cells, SinoCatalog: 40068-V08B
(SEQ ID NO: 38)
MASPAAPRAVSFADNNDITNTNLSRGRGRNPKPRAAPNNTVSWYTGLTQHG
KVPLTFPPGQGVPLNANSTPAQNAGYWRRQDRKINTGNGIKQLAPRWYFYY
TGTGPEAALPFRAVKDGIVWVHEDGATDAPSTFGTRNPNNDSAIVTQFAPGT
KLPKNFHIEGTGGNSQSSSRASSLSRNSSRSSSQGSRSGNSTRGTSPGPSGIGAV
GGDLLYLDLLNRLQALESGKVKQSQPKVITKKDAAAAKNKMRHKRTSTKSF
NMVQAFGLRGPGDLQGNFGDLQLNKLGTEDPRWPQIAELAPTASAFMGMSQ
FKLTHQNNDDHGNPVYFLRYSGAIKLDPKNPNYNKWLELLEQNIDAYKTFPK
KEKKQKAPKEESTDQMSEPPKEQRVQGSITQRTRTRPSVQPGPMIDVNTDAH
HHHHHHHHH
Antigen: MERS-COV_S1,N-(AA1-725,HisTag)_HEK293, Virus: MERS,
Strain: MERS, Uniprot/Genbank: AFS88936.1, Expression: HEK293, SinoCatalog:
40069-V08H
(SEQ ID NO: 39)
YVDVGPDSVKSACIEVDIQQTFFDKTWPRPIDVSKADGIIYPQGRTYSNITITY
QGLFPYQGDHGDMYVYSAGHATGTTPQKLFVANYSQDVKQFANGFVVRIG
AAANSTGTVIISPSTSATIRKIYPAFMLGSSVGNFSDGKMGRFFNHTLVLLPDG
CGTLLRAFYCILEPRSGNHCPAGNSYTSFATYHTPATDCSDGNYNRNASLNSF
KEYFNLRNCTFMYTYNITEDEILEWFGITQTAQGVHLFSSRYVDLYGGNMFQ
FATLPVYDTIKYYSIIPHSIRSIQSDRKAWAAFYVYKLQPLTFLLDFSVDGYIRR
AIDCGFNDLSQLHCSYESFDVESGVYSVSSFEAKPSGSVVEQAEGVECDFSPL
LSGTPPQVYNFKRLVFTNCNYNLTKLLSLFSVNDFTCSQISPAAIASNCYSSLIL
DYFSYPLSMKSDLSVSSAGPISQFNYKQSFSNPTCLILATVPHNLTTITKPLKYS
YINKCSRLLSDDRTEVPQLVNANQYSPCVSIVPSTVWEDGDYYRKQLSPLEG
GGWLVASGSTVAMTEQLQMGFGITVQYGTDTNSVCPKLEFANDTKIASQLG
NCVEYSLYGVSGRGVFQNCTAVGVRQQRFVYDAYQNLVGYYSDDGNYYCL
RACVSVPVSVIYDKETKTHATLFGSVACEHISSTMSQYSRSTRSMLKRRDSTY
GPLQTPVGCVLGLVNSSLFVEAHHHHHHHHHH
Antigen: MERS-COV_S1-RBD,N-(AA367-606,rFcTag), Virus: MERS,
Strain: MERS, Uniprot/Genbank: AFS88936.1, Expression: Insect, Cells,
SinoCatalog: 40071-V31B1
(SEQ ID NO: 40)
EAKPSGSVVEQAEGVECDFSPLLSGTPPQVYNFKRLVFTNCNYNLTKLLSLFS
VNDFTCSQISPAAIASNCYSSLILDYFSYPLSMKSDLSVSSAGPISQFNYKQSFS
NPTCLILATVPHNLTTITKPLKYSYINKCSRLLSDDRTEVPQLVNANQYSPCVS
IVPSTVWEDGDYYRKQLSPLEGGGWLVASGSTVAMTEQLQMGFGITVQYGT
DTNSVCPKLEFANDTKIASQLGNCVEYASKPTCPPPELLGGPSVFIFPPKPKDT
LMISRTPEVTCVVVDVSQDDPEVQFTWYINNEQVRTARPPLREQQFNSTIRVV
STLPIAHQDWLRGKEFKCKVHNKALPAPIEKTISKARGQPLEPKVYTMGPPRE
ELSSRSVSLTCMINGFYPSDISVEWEKNGKAEDNYKTTPAVLDSDGSYFLYSK
LSVPTSEWQRGDVFTCSVMHEALHNHYTQKSISRSPGK
Antigen: MERS-COV, S1-RBD,, N-(AA383-502,, mFc, Tag), Virus: MERS,
Strain: MERS, Uniprot/Genbank: AFS88936.1, Expression: Insect, Cells,
SinoCatalog: 40071-V05B
(SEQ ID NO: 41)
CDFSPLLSGTPPQVYNFKRLVFTNCNYNLTKLLSLFSVNDFTCSQISPAAIASN
CYSSLILDYFSYPLSMKSDLSVSSAGPISQFNYKQSFSNPTCLILATVPHNLTTI
TKPLKYSYINKADDDDKAVPRDSGCKPCICTVPEVSSVFIFPPKPKDVLTITLT
PKVTCVVVDISKDDPEVQFSWFVDDVEVHTAQTQPREEQFNSTFRSVSELPIM
HQDWLNGKEFKCRVNSAAFPAPIEKTISKTKGRPKAPQVYTIPPPKEQMAKD
KVSLTCMITDFFPEDITVEWQWNGQPAENYKNTQPIMDTDGSYFVYSKLNVQ
KSNWEAGNTFTCSVLHEGLHNHHTEKSLSHSPGK
Antigen: MERS-COV_S2, Virus: MERS, Strain: MERS, Uniprot/Genbank:
AFS88936.1, Expression: Insect, Cells, SinoCatalog: 40070-V08B
(SEQ ID NO: 42)
DCKLPLGQSLCALPDTPSTLTPRSVRSVPGEMRLASIAFNHPIQVDQLNSS
YFKLSIPTNFSFGVTQEYIQTTIQKVTVDCKQYVCNGFQKCEQLLREYGQF
CSKINQALHGANLRQDDSVRNLFASVKSSQSSPIIPGFGGDFNLTLLEPVSI
STGSRSARSAIEDLLFDKVTIADPGYMQGYDDCMQQGPASARDLICAQY
VAGYKVLPPLMDVNMEAAYTSSLLGSIAGVGWTAGLSSFAAIPFAQSIFY
RLNGVGITQQVLSENQKLIANKFNQALGAMQTGFTTTNEAFQKVQDAVN
NNAQALSKLASELSNTFGAISASIGDIIQRLDVLEQDAQIDRLINGRLTTLN
AFVAQQLVRSESAALSAQLAKDKVNECVKAQSKRSGFCGQGTHIVSFVV
NAPNGLYFMHVGYYPSNHIEVVSAYGLCDAANPTNCIAPVNGYFIKTNN
TRIVDEWSYTGSSFYAPEPITSLNTKYVAPQVTYQNISTNLPPPLLGNSTGI
DFQDELDEFFKNVSTSIPNFGSLTQINTTLLDLTYEMLSLQQVVKALNESY
IDLKELGNYTYYNKWPAHHHHHHHHHH
Antigen: RSV-A_LA2-94/2013_F, Virus: RSV, Strain: LA2-94/2013,
Uniprot/Genbank: A0A023RA53, Expression: Insect, Cells, SinoCatalog: UN3-1
(SEQ ID NO: 43)
MELPILKTNAITTILAAVTLCFTSSQNITEEFYQSTCSAVSKGYLSALRTGWYT
SVITIELSNIKENKCNGTDAKVKLIKQELDKYKNAVTELQLLMQSSPAANSRA
RRELPRFMNYTLNNTKNTNVTLSKKRKRRFLGFLLGVGSAIASGIAVSKVLHL
EGEVNKIKSALLSTNKAVVSLSNGVSVLTSKVLDLKNYIDKQLLPIVNKQSCSI
SNIETVIEFQQKNNRLLEITREFSVNAGVTTPVSTYMLTNSELLSLINDMPITND
QKKLMSSNVQIVRQQSYSIMSIIKEEVLAYVVQLPLYGVIDTPCWKLHTSPLC
TTNTKEGSNICLTRTDRGWYCDNAGSVSFFPQAETCKVQSNRVFCDTMNSLT
LPSEVNLCNIDIFNPKYDCKIMTSKTDVSSSVITSLGAIVSCYGKTKCTASNKN
RGIIKTFSNGCDYVSNKGVDTVSVGNTLYYVNKQEGKSLYVKGEPIINFYDPL
VFPSDEFDASISQVNEKINQSLAFIRKSDELLHNVNAGKSTTNIMITTIIIVIIVIL
LALIAVGLLLYCKARSTPVTLSKDQLSGINNIAFSN
Antigen: RSV-A_LA2-94/2013_G, Virus: RSV, Strain: LA2-94/2013,
Uniprot/Genbank: A0A076FRQ0, Expression: HEK293, SinoCatalog: UN4-1
(SEQ ID NO: 44)
MSKTKDQRTAKTLERTWDTLNHLLFISSCLYKLNLKSIAQITLSILAMIISTSLII
AAIIFIASANHKVTLTTAIIQDATNQIKNTTPTYLTQNPQLGISFSNLSGTTSQST
TILASTTPSAESTPQSTTVKIKNTTTTQILPSKPTTKQRQNKPQNKPNNDFHFEV
FNFVPCSICSNNPTCWAICKRIPNKKPGKKTTTKPTKKPTLNTTKKDPKPQTTK
PKEVLTTKPTGKPTINTTRTNIRTTLLTSNTKGNPEHTSQEETLHSTTSEGYPSP
SQVYTTSGQEETLHSTTSEGYPSPSQVHTTSEYLSQSLSSSNTTK
Antigen: RSV-B_TH-10526/2014_F, Virus: RSV, Strain: TH-10526/2014,
Uniprot/Genbank: K7WLI9, Expression: Insect, Cells, SinoCatalog: UN3-2
(SEQ ID NO: 45)
MELLIHRSSAIFLTLAINALYLTSSQNITEEFYQSTCSAVSRGYLSALRTGWYTS
VITIELSNIKETKCNGTDTKVKLIKQELDKYKNAVTELQLLMQNTPAVNNRAR
REAPQYMNYTINTTKNLNVSISKKRKRRFLGFLLGVGSAIASGIAVSKVLHLE
GEVNKIKNALQSTNKAVVSLSNGVSVLTSKVLDLKNYINNQLLPIVNQQSCRI
SNIETVIEFQQKNSRLLEITREFSVNAGVTTPLSTYMLTNSELLSLINDMPITND
QKKLMSSNVQIVRQQSYSIMSIIKEEVLAYVVQLPIYGVIDTPCWKLHTSPLCT
TNIKEGSNICLTRTDRGWYCDNAGSVSFFPQADTCKVQSNRVFCDTMNSLTL
PSEVSLCNTDIFNSKYDCKIMTSKTDISSSVITSLGAIVSCYGKTKCTASNKNR
GIIKTFSNGCDYVSNKGVDTVSVGNTLYYVNKLEGKNLYVKGEPIINYYDPL
VFPSDEFDASISQVNEKINQSLAFIRRSDELLHNVNTGKSTTNIMITAIIIVIIVVL
LSLIAIGLLLYCKAKNTPVTLSKDQLSGINNIAFSK
Antigen: RSV-B_B1_G, Virus: RSV, Strain: B1, Uniprot/Genbank:, Expression:
HEK293, SinoCatalog: 13029-V08H
(SEQ ID NO: 46)
HKVTLTTVTVQTIKNHTEKNITTYLTQVPPERVSSSKQPTTTSPIHTNSATTSPN
TKSETHHTTAQTKGRTTTSTQTNKPSTKPRLKNPPKKPKDDYHFEVFNFVPCS
ICGNNQLCKSICKTIPSNKPKKKPTIKPTNKPTTKTTNKRDPKTPAKTTKKETT
TNPTKKPTLTTTERDTSTSQSTVLDTTTLEHTIQQQSLHSTTPENTPNSTQTPTA
SEPSTSNSTQNTQSHA
Antigen: SARS-COV-2_NP, Virus: SARS-COV-2, Strain: 2019-nCoV, Expression:
Insect, Cells, SinoCatalog: 40588-V08B
(SEQ ID NO: 47)
MSDNGPQNQRNAPRITFGGPSDSTGSNQNGERSGARSKQRRPQGLPNNTASW
FTALTQHGKEDLKFPRGQGVPINTNSSPDDQIGYYRRATRRIRGGDGKMKDL
SPRWYFYYLGTGPEAGLPYGANKDGIIWVATEGALNTPKDHIGTRNPANNAA
IVLQLPQGTTLPKGFYAEGSRGGSQASSRSSSRSRNSSRNSTPGSSRGTSPARM
AGNGGDAALALLLLDRLNQLESKMSGKGQQQQGQTVTKKSAAEASKKPRQ
KRTATKAYNVTQAFGRRGPEQTQGNFGDQELIRQGTDYKHWPQIAQFAPSAS
AFFGMSRIGMEVTPSGTWLTYTAAIKLDDKDPNFKDQVILLNKHIDAYKTFPP
TEPKKDKKKKADETQALPQRQKKQQTVTLLPAADLDDFSKQLQQSMSSADS
TQAAHHHHHHHHHH
Antigen: SARS-COV-2_S1, Virus: SARS-COV-2, Strain: SARS-COV-2_S1, Subunit,,
His, Tag,, Baculovirus-Insect, Cells, Expression: HEK293, SinoCatalog: 40591-
V08B1
(SEQ ID NO: 49)
VNLTTRTQLPPAYTNSFTRGVYYPDKVFRSSVLHSTQDLFLPFFSNVTWFHAI
HVSGTNGTKRFDNPVLPFNDGVYFASTEKSNIIRGWIFGTTLDSKTQSLLIVNN
ATNVVIKVCEFQFCNDPFLGVYYHKNNKSWMESEFRVYSSANNCTFEYVSQP
FLMDLEGKQGNFKNLREFVFKNIDGYFKIYSKHTPINLVRDLPQGFSALEPLV
DLPIGINITRFQTLLALHRSYLTPGDSSSGWTAGAAAYYVGYLQPRTFLLKYN
ENGTITDAVDCALDPLSETKCTLKSFTVEKGIYQTSNFRVQPTESIVRFPNITNL
CPFGEVFNATRFASVYAWNRKRISNCVADYSVLYNSASFSTFKCYGVSPTKL
NDLCFTNVYADSFVIRGDEVRQIAPGQTGKIADYNYKLPDDFTGCVIAWNSN
NLDSKVGGNYNYLYRLFRKSNLKPFERDISTEIYQAGSTPCNGVEGFNCYFPL
QSYGFQPTNGVGYQPYRVVVLSFELLHAPATVCGPKKSTNLVKNKCVNFNF
NGLTGTGVLTESNKKFLPFQQFGRDIADTTDAVRDPQTLEILDITPCSFGGVSV
ITPGTNTSNQVAVLYQDVNCTEVPVAIHADQLTPTWRVYSTGSNVFQTRAGC
LIGAEHVNNSYECDIPIGAGICASYQTQTNSPRRARAHHHHHHHHHH
Antigen: SARS-COV-2_S1,(HisTag), Virus: SARS-COV-2, Strain: 2019-nCoV,
Expression: HEK293, SinoCatalog: 40591-V08H
(SEQ ID NO: 50)
VNLTTRTQLPPAYTNSFTRGVYYPDKVFRSSVLHSTQDLFLPFFSNVTWFHAI
HVSGTNGTKRFDNPVLPFNDGVYFASTEKSNIIRGWIFGTTLDSKTQSLLIVNN
ATNVVIKVCEFQFCNDPFLGVYYHKNNKSWMESEFRVYSSANNCTFEYVSQP
FLMDLEGKQGNFKNLREFVFKNIDGYFKIYSKHTPINLVRDLPQGFSALEPLV
DLPIGINITRFQTLLALHRSYLTPGDSSSGWTAGAAAYYVGYLQPRTFLLKYN
ENGTITDAVDCALDPLSETKCTLKSFTVEKGIYQTSNFRVQPTESIVRFPNITNL
CPFGEVFNATRFASVYAWNRKRISNCVADYSVLYNSASFSTFKCYGVSPTKL
NDLCFTNVYADSFVIRGDEVRQIAPGQTGKIADYNYKLPDDFTGCVIAWNSN
NLDSKVGGNYNYLYRLFRKSNLKPFERDISTEIYQAGSTPCNGVEGFNCYFPL
QSYGFQPTNGVGYQPYRVVVLSFELLHAPATVCGPKKSTNLVKNKCVNFNF
NGLTGTGVLTESNKKFLPFQQFGRDIADTTDAVRDPQTLEILDITPCSFGGVSV
ITPGTNTSNQVAVLYQDVNCTEVPVAIHADQLTPTWRVYSTGSNVFQTRAGC
LIGAEHVNNSYECDIPIGAGICASYQTQTNSPRRARAHHHHHHHHHH
Antigen: SARS-COV-2_S1,(mFcTag), Virus: SARS-COV-2, Strain: 2019-nCoV,
Expression: HEK293, SinoCatalog: 40591-V05H1
(SEQ ID NO: 51)
VNLTTRTQLPPAYTNSFTRGVYYPDKVFRSSVLHSTQDLFLPFFSNVTWFHAI
HVSGTNGTKRFDNPVLPFNDGVYFASTEKSNIIRGWIFGTTLDSKTQSLLIVNN
ATNVVIKVCEFQFCNDPFLGVYYHKNNKSWMESEFRVYSSANNCTFEYVSQP
FLMDLEGKQGNFKNLREFVFKNIDGYFKIYSKHTPINLVRDLPQGFSALEPLV
DLPIGINITRFQTLLALHRSYLTPGDSSSGWTAGAAAYYVGYLQPRTFLLKYN
ENGTITDAVDCALDPLSETKCTLKSFTVEKGIYQTSNFRVQPTESIVRFPNITNL
CPFGEVFNATRFASVYAWNRKRISNCVADYSVLYNSASFSTFKCYGVSPTKL
NDLCFTNVYADSFVIRGDEVRQIAPGQTGKIADYNYKLPDDFTGCVIAWNSN
NLDSKVGGNYNYLYRLFRKSNLKPFERDISTEIYQAGSTPCNGVEGFNCYFPL
QSYGFQPTNGVGYQPYRVVVLSFELLHAPATVCGPKKSTNLVKNKCVNFNF
NGLTGTGVLTESNKKFLPFQQFGRDIADTTDAVRDPQTLEILDITPCSFGGVSV
ITPGTNTSNQVAVLYQDVNCTEVPVAIHADQLTPTWRVYSTGSNVFQTRAGC
LIGAEHVNNSYECDIPIGAGICASYQTQTNSPRRARADDDDKAVPRDSGCKPC
ICTVPEVSSVFIFPPKPKDVLTITLTPKVTCVVVDISKDDPEVQFSWFVDDVEV
HTAQTQPREEQFNSTFRSVSELPIMHQDWLNGKEFKCRVNSAAFPAPIEKTIS
KTKGRPKAPQVYTIPPPKEQMAKDKVSLTCMITDFFPEDITVEWQWNGQPAE
NYKNTQPIMDTDGSYFVYSKLNVQKSNWEAGNTFTCSVLHEGLHNHHTEKS
LSHSPGK
Antigen: SARS-COV-2_S1-RBD, Virus: SARS-COV-2, Strain: 2019-nCoV,
Expression: HEK293, SinoCatalog: 40592-V05H
(SEQ ID NO: 52)
RVQPTESIVRFPNITNLCPFGEVFNATRFASVYAWNRKRISNCVADYSVLYNS
ASFSTFKCYGVSPTKLNDLCFTNVYADSFVIRGDEVRQIAPGQTGKIADYNYK
LPDDFTGCVIAWNSNNLDSKVGGNYNYLYRLFRKSNLKPFERDISTEIYQAGS
TPCNGVEGFNCYFPLQSYGFQPTNGVGYQPYRVVVLSFELLHAPATVCGPKK
STNLVKNKCVNFADDDDKAVPRDSGCKPCICTVPEVSSVFIFPPKPKDVLTITL
TPKVTCVVVDISKDDPEVQFSWFVDDVEVHTAQTQPREEQFNSTFRSVSELPI
MHQDWLNGKEFKCRVNSAAFPAPIEKTISKTKGRPKAPQVYTIPPPKEQMAK
DKVSLTCMITDFFPEDITVEWQWNGQPAENYKNTQPIMDTDGSYFVYSKLNV
QKSNWEAGNTFTCSVLHEGLHNHHTEKSLSHSPGK
Antigen: SARS-COV-2_S1 + S2, Virus: SARS-COV-2, Strain: 2019-nCoV, Expression:
Insect, Cells, SinoCatalog: 40589-V08B1
(SEQ ID NO: 53)
VNLTTRTQLPPAYTNSFTRGVYYPDKVFRSSVLHSTQDLFLPFFSNVTWFHAI
HVSGTNGTKRFDNPVLPFNDGVYFASTEKSNIIRGWIFGTTLDSKTQSLLIVNN
ATNVVIKVCEFQFCNDPFLGVYYHKNNKSWMESEFRVYSSANNCTFEYVSQP
FLMDLEGKQGNFKNLREFVFKNIDGYFKIYSKHTPINLVRDLPQGFSALEPLV
DLPIGINITRFQTLLALHRSYLTPGDSSSGWTAGAAAYYVGYLQPRTFLLKYN
ENGTITDAVDCALDPLSETKCTLKSFTVEKGIYQTSNFRVQPTESIVRFPNITNL
CPFGEVFNATRFASVYAWNRKRISNCVADYSVLYNSASFSTFKCYGVSPTKL
NDLCFTNVYADSFVIRGDEVRQIAPGQTGKIADYNYKLPDDFTGCVIAWNSN
NLDSKVGGNYNYLYRLFRKSNLKPFERDISTEIYQAGSTPCNGVEGFNCYFPL
QSYGFQPTNGVGYQPYRVVVLSFELLHAPATVCGPKKSTNLVKNKCVNFNF
NGLTGTGVLTESNKKFLPFQQFGRDIADTTDAVRDPQTLEILDITPCSFGGVSV
ITPGTNTSNQVAVLYQDVNCTEVPVAIHADQLTPTWRVYSTGSNVFQTRAGC
LIGAEHVNNSYECDIPIGAGICASYQTQTNSPRRARSVASQSIIAYTMSLGAEN
SVAYSNNSIAIPTNFTISVTTEILPVSMTKTSVDCTMYICGDSTECSNLLLQYGS
FCTQLNRALTGIAVEQDKNTQEVFAQVKQIYKTPPIKDFGGFNFSQILPDPSKP
SKRSFIEDLLFNKVTLADAGFIKQYGDCLGDIAARDLICAQKFNGLTVLPPLLT
DEMIAQYTSALLAGTITSGWTFGAGAALQIPFAMQMAYRFNGIGVTQNVLYE
NQKLIANQFNSAIGKIQDSLSSTASALGKLQDVVNQNAQALNTLVKQLSSNFG
AISSVLNDILSRLDKVEAEVQIDRLITGRLQSLQTYVTQQLIRAAEIRASANLA
ATKMSECVLGQSKRVDFCGKGYHLMSFPQSAPHGVVFLHVTYVPAQEKNFT
TAPAICHDGKAHFPREGVFVSNGTHWFVTQRNFYEPQUITTDNTFVSGNCDVV
IGIVNNTVYDPLQPELDSFKEELDKYFKNHTSPDVDLGDISGINASVVNIQKEI
DRLNEVAKNLNESLIDLQELGKYEQYIKWPAHHHHHHHHHH
Antigen: SARS-COV-2_S2, Virus: SARS-COV-2, Strain: 2019-nCoV, Expression:
Insect, Cells, SinoCatalog: 40590-V08B
(SEQ ID NO: 54)
SVASQSIIAYTMSLGAENSVAYSNNSIAIPTNFTISVTTEILPVSMTKTSVDCTM
YICGDSTECSNLLLQYGSFCTQLNRALTGIAVEQDKNTQEVFAQVKQIYKTPPI
KDFGGFNFSQILPDPSKPSKRSFIEDLLFNKVTLADAGFIKQYGDCLGDIAARD
LICAQKFNGLTVLPPLLTDEMIAQYTSALLAGTITSGWTFGAGAALQIPFAMQ
MAYRFNGIGVTQNVLYENQKLIANQFNSAIGKIQDSLSSTASALGKLQDVVN
QNAQALNTLVKQLSSNFGAISSVLNDILSRLDKVEAEVQIDRLITGRLQSLQTY
VTQQLIRAAEIRASANLAATKMSECVLGQSKRVDFCGKGYHLMSFPQSAPHG
VVFLHVTYVPAQEKNFTTAPAICHDGKAHFPREGVFVSNGTHWFVTQRNFYE
PQIITTDNTFVSGNCDVVIGIVNNTVYDPLQPELDSFKEELDKYFKNHTSPDVD
LGDISGINASVVNIQKEIDRLNEVAKNLNESLIDLQELGKYEQYIKWPAHHHH
HHHHHH
Antigen: SARS-COV-2, (2019-nCoV), Spike, RBD-His, Recombinant, Protein,,
Baculovirus-Insect, Cells, Virus: SARS-COV-2, Strain: 2019-nCoV, Expression:
Insect, Cells, SinoCatalog: 40592-V08B
(SEQ ID NO: 55)
RVQPTESIVRFPNITNLCPFGEVFNATRFASVYAWNRKRISNCVADYSVLYNS
ASFSTFKCYGVSPTKLNDLCFTNVYADSFVIRGDEVRQIAPGQTGKIADYNYK
LPDDFTGCVIAWNSNNLDSKVGGNYNYLYRLFRKSNLKPFERDISTEIYQAGS
TPCNGVEGFNCYFPLQSYGFQPTNGVGYQPYRVVVLSFELLHAPATVCGPKK
STNLVKNKCVNFAHHHHHHHHHH
Antigen: SARS-COV-2, (2019-nCoV), Spike, RBD-His, Recombinant, Protein,,
HEK293, Virus: SARS-COV-2, Strain: 2019-nCoV, Uniprot/Genbank:, Expression:
HEK293, SinoCatalog: 40592-V08H
(SEQ ID NO: 56)
RVQPTESIVRFPNITNLCPFGEVFNATRFASVYAWNRKRISNCVADYSVLYNS
ASFSTFKCYGVSPTKLNDLCFTNVYADSFVIRGDEVRQIAPGQTGKIADYNYK
LPDDFTGCVIAWNSNNLDSKVGGNYNYLYRLFRKSNLKPFERDISTEIYQAGS
TPCNGVEGFNCYFPLQSYGFQPTNGVGYQPYRVVVLSFELLHAPATVCGPKK
STNLVKNKCVNF
Antigen: SARS-COV-2, (2019-nCoV), Spike, RBD-rFc, Recombinant, Protein, Virus:
SARS-COV-2, Strain: 2019-nCoV, Uniprot/Genbank:, Expression: HEK293,
SinoCatalog: 40592-V31H
(SEQ ID NO: 57)
RVQPTESIVRFPNITNLCPFGEVFNATRFASVYAWNRKRISNCVADYSVLYNS
ASFSTFKCYGVSPTKLNDLCFTNVYADSFVIRGDEVRQIAPGQTGKIADYNYK
LPDDFTGCVIAWNSNNLDSKVGGNYNYLYRLFRKSNLKPFERDISTEIYQAGS
TPCNGVEGFNCYFPLQSYGFQPTNGVGYQPYRVVVLSFELLHAPATVCGPKK
STNLVKNKCVNF
Antigen: SARS-COV_PLpro, Virus: SARS-COV, Strain: SARS, Uniprot/Genbank:
AAX16193.1, Expression: E., coli, SinoCatalog: 40524-V08E
(SEQ ID NO: 58)
EVKTIKVFTTVDNTNLHTQLVDMSMTYGQQFGPTYLDGADVTKIKPHVNHE
GKTFFVLPSDDTLRSEAFEYYHTLDESFLGRYMSALNHTKKWKFPQVGGLTS
IKWADNNCYLSSVLLALQQLEVKFNAPALQEAYYRARAGDAANFCALILAY
SNKTVGELGDVRETMTHLLQHANLESAKRVLNVVCKHCGQKTTTLTGVEAV
MYMGTLSYDNLKTGVSIPCVCGRDATQYLVQQESSFVMMSAPPAEYKLQQG
TFLCANEYTGNYQCGHYTHITAKETLYRIDGAHLTKMLEYKGPVTDVFYKET
SYTTTIKPVSYKLDGVTYTEIEPKLDGYYKKDNAYYTEQPID
Antigen: SARS-COV_S1,(HisTag), Virus: SARS-COV, Strain: SARS,
Uniprot/Genbank: AAX16192.1, Expression: Insect, Cells, SinoCatalog: 40150-
V08B1
(SEQ ID NO: 59)
SDLDRCTTFDDVQAPNYTQHTSSMRGVYYPDEIFRSDTLYLTQDLFLPFYSNV
TGFHTINHTFDNPVIPFKDGIYFAATEKSNVVRGWVFGSTMNNKSQSVIIINNS
TNVVIRACNFELCDNPFFAVSKPMGTQTHTMIFDNAFNCTFEYISDAFSLDVS
EKSGNFKHLREFVFKNKDGFLYVYKGYQPIDVVRDLPSGFNTLKPIFKLPLGI
NITNFRAILTAFSPAQDTWGTSAAAYFVGYLKPTTFMLKYDENGTITDAVDCS
QNPLAELKCSVKSFEIDKGIYQTSNFRVVPSGDVVRFPNITNLCPFGEVFNATK
FPSVYAWERKKISNCVADYSVLYNSTFFSTFKCYGVSATKLNDLCFSNVYAD
SFVVKGDDVRQIAPGQTGVIADYNYKLPDDFMGCVLAWNTRNIDATSTGNY
NYKYRYLRHGKLRPFERDISNVPFSPDGKPCTPPALNCYWPLNDYGFYTTTGI
GYQPYRVVVLSFELLNAPATVCGPKLSTDLIKNQCVNFNFNGLTGTGVLTPSS
KRFQPFQQFGRDVSDFTDSVRDPKTSEILDISPCSFGGVSVITPGTNASSEVAV
LYQDVNCTDVSTAIHADQLTPAWRIYSTGNNVFQTQAGCLIGAEHVDTSYEC
DIPIGAGICASYHTVSLLRAHHHHHHHHHH
Antigen: SARS-COV_S1-RBD, (HisTag), Virus: SARS-COV, Strain: SARS,
Uniprot/Genbank: AAX16192.1, Expression: Insect, Cells, SinoCatalog: 40150-
V08B2
(SEQ ID NO: 60)
RVVPSGDVVRFPNITNLCPFGEVFNATKFPSVYAWERKKISNCVADYSVLYN
STFFSTFKCYGVSATKLNDLCFSNVYADSFVVKGDDVRQIAPGQTGVIADYN
YKLPDDFMGCVLAWNTRNIDATSTGNYNYKYRYLRHGKLRPFERDISNVPFS
PDGKPCTPPALNCYWPLNDYGFYTTTGIGYQPYRVVVLSFELLNAPATVCGP
KLSTDLIKNQCVNFAHHHHHHHHHH
Antigen: SARS-COV_S1-RBD,rFcTag, Virus: SARS-COV, Strain: SARS,
Uniprot/Genbank: AAX16192.1, Expression: Insect, Cells, SinoCatalog: 40150-
V31B2
(SEQ ID NO: 61)
RVVPSGDVVRFPNITNLCPFGEVFNATKFPSVYAWERKKISNCVADYSVLYN
STFFSTFKCYGVSATKLNDLCFSNVYADSFVVKGDDVRQIAPGQTGVIADYN
YKLPDDFMGCVLAWNTRNIDATSTGNYNYKYRYLRHGKLRPFERDISNVPFS
PDGKPCTPPALNCYWPLNDYGFYTTTGIGYQPYRVVVLSFELLNAPATVCGP
KLSTDLIKNQCVNFASKPTCPPPELLGGPSVFIFPPKPKDTLMISRTPEVTCVVV
DVSQDDPEVQFTWYINNEQVRTARPPLREQQFNSTIRVVSTLPIAHQDWLRG
KEFKCKVHNKALPAPIEKTISKARGQPLEPKVYTMGPPREELSSRSVSLTCMIN
GFYPSDISVEWEKNGKAEDNYKTTPAVLDSDGSYFLYSKLSVPTSEWQRGDV
FTCSVMHEALHNHYTQKSISRSPGK

### Coronavirus Antigen Array (CoVAM) Data Analysis and Reporting

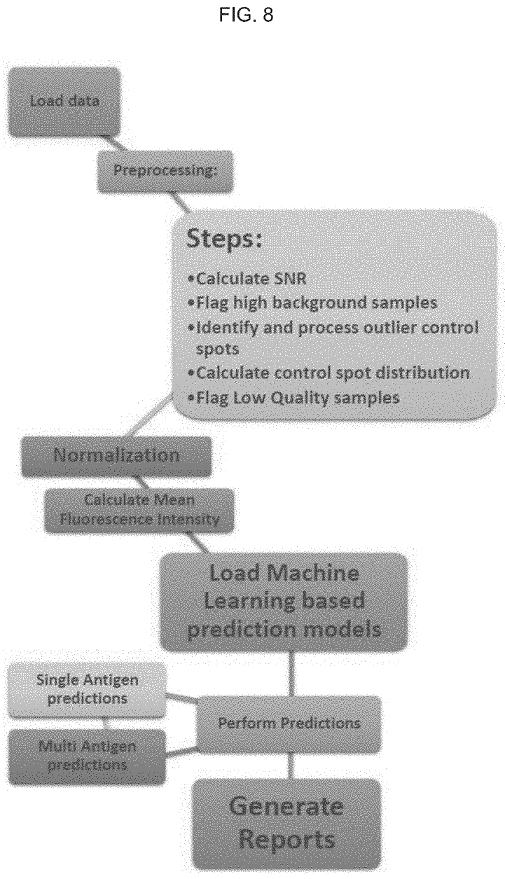

In alternative embodiments, provided are data analysis and collating programs used to analyze the data arising from use of the products of manufacture, including biochips, microarrays and arrays, as provided herein (the so-called “COVAM array”) to detect the presence of a virus in a biological sample, and to generate the individual reports, as described for example in FIG. 8.

In alternative embodiments, provided are data analysis and collating software that encodes an analysis algorithm that takes the input of raw data generated by an antigen microarray as provided herein, normalizes the data to allow comparisons between samples and minimize batch-to-batch variability, and applies a prediction model to determine prior infection status. The prediction model is constructed by selecting reactive antigens that meet a threshold for signal-to-noise ratio and testing all weighted combinations of these antigens for predictive performance using positive controls with confirmed infection and negative controls without infection. The testing for predictive performance occurs via a split testing and validation set approach using all positives and negatives in order to select the optimal weighted combination of antigens. A cutoff value for this optimal weighted combination is selected to maximize specificity. The overall performance of the weighted antigen combination with the selected cutoff is determined by applying to all positive and negative controls. Reports are generated for each specimen that include the overall prediction for IgG and IgM and individual antigen reactivity compared to positive and negative controls.

In alternative embodiments, provided are data analysis and collating software programs comprising:

### Step 1: Data Pre-Processing

In alternative embodiments, the first step of the analysis is importing all data into the R environment. The sample set containing the known negative and known positive controls, here named “Control Set”, is loaded separately from the sample set being analyses.

Following this step, to prevent errors when addressing specific columns, or samples, all spaces are removed both from the column names from all data sets imported, as well as from the Unique sample IDs reference from the meta data files.

On the data processing steps, the following are performed:

From the raw data, the signal to noise ratio (SNR) is calculated. The SNR is calculated as the median signal intensity of a given spot divided by the background signal of the vicinity surrounding area. For the quality check purposes, the mean SNR is calculated only for spots with MFI over 20,000. Samples with a mean SNR below 2 are flagged for further visual inspection or for re-probing.

After calculating the mean SNR, the control spots are then assessed. First, for each sample, and each antigen (printed in triplicates), the first and third quartile as well as interquartile range (IQR) are calculated for the control spots. The upper MFI limit of 1.5 times the IQR over the third quartile and a lower limit of 1.5 times the IQR below the first quartile are defined. Spots outside this range are removed and replaced with the mean MFI of the remaining replicates of the spot.

Next, a similar approach is applied to flag samples for which the overall control spots distribution is out of range (2*IQR+third Quartile for the upper limit and first quartile−2*IQR for the lower limit). For this, all controls spots of a given sample are used. Out of range samples are flagged for further visual inspection or re-probing.

Finally, the printing buffer background reactivity is subtracted from each spot and the samples are normalized.

### Step 2: Normalization

In alternative embodiments, data normalization is performed in two steps. First The control spots are normalized against the training set using the Quantile Normalization method. This allows to calculate a normalization factor that will be used to rescale the data to match the training set and preserving the individual reactivity diversity. After normalizing the control spots, their sum is calculated. A rescaling factor is calculated by dividing the sum of the normalized control spots of the training set by the sum of the normalized control spots of each sample. The resulting factor is then multiplied by the reactivity of each spot resulting in a rescaled data frame. The mean reactivity of the normalized data is then calculated.

### Step 3a: Prediction Models

In alternative embodiments, prediction models are constructed previous to the sample analysis using a sample set composed by samples with known diagnosis for COVID-19.

Previous to the sample analysis, the prediction models were constructed using a sample set composed by samples with known diagnosis for COVID-19. These samples are both Negative controls (samples collected before the pandemic) and Positive controls (Samples from individuals diagnosed for COVID-19 by PCR). This control set is referred to as “Training Set”.

The construction of the prediction models was (or can be) performed as following.
1. Data is pre-processed and normalized as described above.
2. The reference data set was decomposed into a vector using the function ‘unmatrix’ from the package gData (version 2.18.0).
3. A mixture model is calculated for the vector using the function ‘normalmixEM’ from the package ‘mixtools’ (version 1.2.0).
4. A cutoff is then calculated as 3 standard deviations over the mean of the negative signal curve.
5. Wilcox test for each antigen was performed comparing the positive controls and negatives control, considering significant, antigens with p <0.05.

Following the selection of seropositive antigens, an optimal predictive combination of these antigens was selected. (that left us with 7 antigens as seropositive for IgG, and 8 for IgM).

In alternative embodiments, the selection can be, or was, performed as follows:
1. For every possible combination of the seropositive SARS-CoV-2 antigens from 1 all (7 for IgG and 8 for IgM), the reference set was randomly divided into a training and a testing sets at a 70%/30% ratio.
2. A logistic regression was generated using the reference set. The regression was generated using the function ‘glm’ of the ‘stats’ package (version 4.0.0). and a ROC curve was calculated (package pROC version 1.16.2).
3. The optimal coordinates of the ROC curve were obtained based on the ‘youden index’, by prioritizing the specificity.
4. The coordinates were obtained using the function ‘coords’ from the pROC library. The coordinates are obtained in a table format with each row containing a regression threshold and its related specificity and sensitivity.
5. The coordinates were then subset to represent specificities of 0.95 or higher. A threshold was then defined as the threshold on the coordinate with the highest specificity on the subset.
6. A logistic regression was then calculated using the testing set and each sample classified as negative or positive by comparison with the threshold.
7. A confusion matrix was calculated by comparing the predicted outcomes and the known classifications (“known negative” or “Known positive”) and the prediction specificity and sensitivity stored into a vector.
8. This analysis was repeated 1000 times and the sensitivity and sensitivity calculated as the mean predicted performance of all repetitions. The performance outcome for each antigen combination was analyzed and a selection of the best performing combinations was made based on the specificity and sensitivity. The selected candidates were then tested using the full reference sample set. The test was performed as follows:
1. A logistic regression for each antigen combination candidate using the full reference set. Then a ROC curve was calculated and the coordinate table with all curve points was obtained.
2. The coordinates of each candidate were compared in order to select the candidate with the highest sensitivity, given a fixed specificity of 1 (100%).

In addition to the logistic regression model, a Random Forest model was constructed using all reactive antigens.

### Step 3 b: Reports

In alternative embodiments, a prediction for each single SARS-CoV-2 antigen for every sample for both IgG, and IgM is performed.

In alternative embodiments, after Data Normalization, the predictions models, constructed as described above, are loaded and reactivity predictions are performed using, for example, Random Forest and Logistic Regression for the multi antigen combinations. In addition to the multi antigen predictions, a prediction for each single SARS-CoV-2 antigen was performed for every sample, for both IgG, and IgM.

In alternative embodiments, these predictions were or can be performed using the threshold calculated using the optimal ‘youden’ index. Every sample can be classified as reactive or not reactive for each single SARS-CoV-2 antigen.

In alternative embodiments, the report phase consists of the output of single pdf files with the individual subject predictions and interpretation. The file consists on a brief explanation of the array on the first page, as well as some information on the performance of the array with the current settings. In alternative embodiments, on the first page there can be a short disclaimer of the scope and limitations of the assay.

In alternative embodiments, the second page can consist of a table for all the SARS-CoV-2 antigens with their ROC predictions. These predictions are for a qualitative understanding of one's reactivity and may not directly correlate with the multi antigen prediction.

In alternative embodiments, the Multi antigen prediction, or the sample classification into the three reactive groups, is presented, for example, on a short table displaying the prediction of IgG and IgM separately.

In alternative embodiments, the overall sero-reactivity of the sample to all antigens is depicted on two graphs on the second page: one showing the reactivity for IgG and one for IgM.

In alternative embodiments, on each graph, the individual' s reactivity is represented, for example, as dots with its standard errors. In alternative embodiments, for reference: a red line representing the positive control mean reactivity with its confidence interval, as well as a blue line representing the negative controls mean reactivity with its confidence interval, are also plotted.

### Computers and Storage Memory Systems

In alternative embodiments, products of manufacture, computer-executed methods or software systems, multiplexed systems and methods as provided herein use apparatus such as computers and storage memory systems for performing the operations as provided herein. This apparatus may be specially constructed for the required purposes, or it may comprise a general-purpose computer selectively activated or reconfigured by a computer program stored in the computer. Such a computer program may be stored in a computer readable storage medium, such as, but not limited to, any type of disk including floppy disks, optical disks, CD-ROMs, and magnetic-optical disks, read-only memories (ROMs), random access memories (RAMs), EPROMs, EEPROMs, magnetic or optical cards, or any type of media suitable for storing electronic instructions.

The algorithms and displays used to practice products of manufacture, computer-executed methods or software systems, multiplexed systems and methods as provided herein are not inherently related to any particular computer or other apparatus. Various general-purpose systems may be used with programs in accordance with the teachings herein, or it may prove convenient to construct a more specialized apparatus to perform the method steps. The structure for a variety of these systems will appear from the description provided herein. In addition, embodiments provided herein are not described with reference to any particular programming language. It will be appreciated that a variety of programming languages may be used to implement and practice methods and systems as described herein.

In alternative embodiments, data generated and processed by components of products of manufacture, computer-executed methods or software systems, multiplexed systems and methods as provided herein, include generated data and programs used to practice embodiments as provided herein, are stored and processed using a machine-readable medium, which can includes any mechanism for storing or transmitting information in a form readable by a machine, for example, a computer. For example, a machine-readable medium includes a machine-readable storage medium (for example, read only memory (“ROM”), random access memory (“RAM”), magnetic disk storage media, optical storage media, flash memory devices, etc.), a machine-readable transmission medium (electrical, optical, acoustical or other form of propagated signals, for example, carrier waves, infrared signals, digital signals, and the like.

In alternative embodiments, programs used to process methods and/or multiplexed systems as provided herein are cloud-based and use wireless systems to communicate (for example, device-to-device (D2D) connectability) with a user (for example, an individual being treated using systems or methods as provided herein) and/or an operator (for example, a person monitoring and/or administering methods or 20 systems as provided herein as they are being practiced, for example, as described in U.S. Pat. No. 10,834,769, which teaches methods by one or more processors for managing a wireless communication network and device-to-device (D2D) connectability.

In alternative embodiments, computer-executed methods or software systems as provided herein use cloud computing to enabling convenient, on-demand network access to a shared pool of configurable computing resources (for example, networks, network bandwidth, servers, processing, memory, storage, applications, virtual machines, and services) that can be rapidly provisioned and released with minimal management effort or interaction with a user or manager of systems or methods as provided herein.

In alternative embodiments, provided herein is a non-transitory, machine-readable medium, comprising executable instructions that, when executed by a processing system including a processor, facilitate performance of operations, the operations comprising a program used to practice computer-executed methods or software systems as provided herein.

In alternative embodiments, provided are computer-implemented methods for implementing the analysis algorithm as provided herein, for example, computer-implemented methods that can take the input of raw data generated by an antigen microarray as provided herein, normalize the data to allow comparisons between samples and minimize batch-to-batch variability, and apply to a prediction model to determine prior infection status. In alternative embodiments, provided are computer program products for processing data, the computer program product comprising computer-executable logic contained on a computer-readable medium and configured for implementing and practicing computer-implemented methods as provided herein. In alternative embodiments, provided are Graphical User Interface (GUI) computer program products configured for implementing and practicing computer-implemented methods as provided herein. In alternative embodiments, provided are computer systems comprising a processor and a data storage device wherein said data storage device has stored thereon: a computer program product for implementing a computer-implemented method as provided herein. In alternative embodiments, provided are non-transitory memory medium or computer-readable storage medium comprising program instructions for running, processing and/or implementing a computer-implemented method as provided herein. In alternative embodiments, provided are computer program storage devices or computer or equivalent electronic systems embodied on a tangible computer readable medium comprising a computer-implemented method as provided herein.

### Coronavirus Vaccines

In alternative embodiments, provided are pharmaceutical formulations or vaccines comprising a peptide or polypeptide derived from the coronavirus Si and/or the coronavirus RBD domain of the coronavirus spike protein, wherein optionally the coronavirus is the strain COVID-19.

In alternative embodiments, pharmaceutical formulations or vaccines comprising a peptide or polypeptide derived from the coronavirus S1 and/or the coronavirus RBD domain of the coronavirus spike protein are formulated and administered using any formulations, protocols or techniques known in the art, for example, pharmaceutical formulations or vaccines as provided herein can be administered as peptides, or can be administered in the form of nucleic acids that encode the immunogenic peptides or proteins.

For example, in alternative embodiments the immunogen-encoding nucleic acid can be a DNA encoding one or more immunogenic peptides or proteins, and the DNA can be carried in an expression vehicle such as a viral vector, for example an adenovirus vector such as an Ad5 or adeno-associated vector (AAV). In alternative embodiments, recombinant adenoviruses as used in vaccines as provided herein can be as described in U.S. patent application no. US 20200399323 A1, which describes for example recombinant adenoviruses including a deletion in or of the E1 region or any deletion that renders the virus replication-defective, for example, the replication-defective virus can include a deletion in one or more of the E1, E3, and/or E4 regions; or, can be as described in U.S. patent application no. US 20190382793 A1, which described how to make recombinant adenoviruses for gene therapy.

In alternative embodiments, the immunogen-encoding nucleic acid can be an RNA, for example, mRNA, which can be formulated in a lipid formulation or a liposome and injected for example intramuscularly (IM), for example using formulations and methods as described in U.S. patent application no. US 20210046173 A1, which describes delivering to a subject (for example, via intramuscular administration) an immunogenic composition that comprises a RNA (for example, mRNA) that comprises an open reading frame (ORF) that comprises (or consists of, or consists essentially of) an immunogenic or antigenic sequence as provided herein; wherein optionally the RNA (or the DNA-carrying expression vehicle) is formulated in a liposome, or a lipid nanoparticle (LNP), or nanoliposome, that comprises: non-cationic lipids comprise a mixture of cholesterol and DSPC, or a PEG-lipid, or PEG-modified lipid, or LNP, or an ionizable cationic lipid; or a mixture of (13Z,16Z)-N,N-dimethyl-2-nonylhenicosa-12,15-dien-1-amine, cholesterol, DSPC, and PEG-2000 DMG. In alternative embodiments, the PEG-lipid is 1,2-Dimyristoyl-sn-glycerol methoxypolyethylene glycol (PEG-DMG), PEG-disteryl glycerol (PEG-DSG), PEG-dipalmetoleyl, PEG-dioleyl, PEG-distearyl, PEG-diacylglycamide (PEG-DAG), PEG-dipalmitoyl phosphatidylethanolamine (PEG-DPPE), or PEG-1,2-dimyristyloxlpropyl-3-amine (PEG-c-DMA), or, the PEG-lipid is PEG coupled to dimyristoylglycerol (PEG-DMG). In alternative embodiments, the LNP comprises 20-99.8 mole % ionizable cationic lipids, 0.1-65 mole % non-cationic lipids, and 0.1-20 mole % PEG-lipid. In alternative embodiments, the LNP comprises an ionizable cationic lipid selected from the group consisting of (2S)-1-({6-[(3))-cholest-5-en-3-yloxy]hexyl}oxy)-N,N-dimethyl-3-[(9 Z)-octadec-9-en-1-yloxy]propan-2-amine; (13Z,16Z)-N,N-dimethyl-3-nonyldocosa-13,16-dien-1-amine; and N,N-dimethyl-1-[(1S,2R)-2-octylcyclopropyl]heptadecan-8-amine; or a pharmaceutically acceptable salt thereof, or a stereoisomer of any of the foregoing. In alternative embodiments, the PEG modified lipid comprises a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof. In alternative embodiments, the ionizable cationic lipid comprises: 2,2-dilinoleyl-4-dimethylaminoethyl-[1,3]-dioxolane (DLin-KC2-DMA), dilinoleyl-methyl-4-dimethylaminobutyrate (DLin-MC3-DMA), di((Z)-non-2-en-1-yl) 9-((4-(dimethyl amino)butanoyl)oxy) heptadecanedioate (L319), (13Z,16Z)-N,N-dimethyl-3-nonyldocosa-13,16-dien-1-amine, (12Z,15Z)-N,N-dimethyl-2-nonylhenicosa-12,15-dien-1-amine, and N,N-dimethyl-1-[(1S,2R)-2-octylcyclopropyl]heptadecan-8-amine. In one embodiment, the lipid is (13Z,16Z)-N,N-dimethyl-3-nonyldocosa-13,16-dien-1-amine or N,N-dimethyl-1-[(1S,2R)-2-octylcyclopropyl]heptadecan-8-amine, each of which are described in PCT/US2011/052328, the entire contents of which are hereby incorporated by reference. In some embodiments, a non-cationic lipid of the disclosure comprises 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2 cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, or mixtures thereof.

In alternative embodiments, the vaccine comprises all of, or an antigenic fragment of, the coronavirus is the strain COVID-19 surface glycoprotein (the S1 Domain (Val16 to Arg685) is represented in bold blue and italicized characters and the RBD subdomain (Arg319 to Phe541) in bold blue and underlined characters):

(SEQ ID NO: 2)
MFVFLVLLPLVSSQCVNLTTRTQLPPAYINSFTRGVYYPDKVFRSSVLH
STQDLFLPFFSNVTWFHAIHVSGTNGTKRFDNPVLPFNDGVYFASTEKS
NIIRGWIFGTTLDSKTQSLLIVNNATNVVIKVCEFQFCNDPFLGVYYHI
KNNKSWMESEFRVYSSANNCTFEYVSQPFLMDLEGKQGNFKNLREFVFK
NIDGYFKIYSKHTPINLVRDLPQGFSALEPLVDLPIGINITRFQTLLAL
HRSYLTPGDSSSGWTAGAAAYYVGYLQPRTFLLKYNENGTITDAVDCAL
DPLSETKCTLKSFTVEKGIYQTSNFRVQPTESIVRFPNITNLCPFGEVF
NATRFASVYAWNRKRISNCVADYSVLYNSASFSTFKCYGVSPTKLNDLC
FTNYYADSFVIRGDEVRQIAPGQTGKIADYNYKTPCNGVEGFNCYFPLQ
SYGFQPTNGVGYQPYRVVVLSFELLHAPATVCGPKKSTNLVKNKCVNFN
FNGLTGTGVLTESNKKFLPFQQFGRDIADTTDAVRDPQTLEILDITPCS
FGGVSVITPGTNTSNQVAVLYQDVNCTEVPVAIHADQLTPTWRVYSTGS
NVFQTRAGCLIGAEHVNNSYECDIPIGAGICASYQTQTNSPRRARSVAS
QSIIAYTMSLGAENSVAYSNNSIAIPTNFTISVTTEILPVSMTKTSVDC
TMYICGDSTECSNLLLQYGSFCTQLNRALTGIAVEQDKNTQEVFAQVKQ
IYKTPPIKDFGGFNFSQILPDPSKPSKRSFIEDLLFNKVTLADAGFIKQ
YGDCLGDIAARDLICAQKFNGLTVLPPLLTDEMIAQYTSALLAGTITSG
WTFGAGAALQIPFAMQMAYRFNGIGVTQNVLYENQKLIANQFNSAIGKI
QDSLSSTASALGKLQDVVNQNAQALNTLVKQLSSNFGAISSVLNDILSR
LDKVEAEVQIDRLITGRLQSLQTYVTQQLIRAAEIRASANLAATKMSEC
VLGQSKRVDFCGKGYHLMSFPQSAPHGVVFLHVTYVPAQEKNFTTAPAI
CHDGKAHFPREGVFVSNGTHWFVTQRNFYEPQIITTDNTFVSGNCDVVI
GIVNNTVYDPLQPELDSFKEELDKYFKNHTSPDVDLGDISGINASVVNI
QKEIDRLNEVAKNLNESLIDLQELGKYEQYIKWPWYIWLGFIAGLIAIV
MVTIMLCCMTSCCSCLKGCCSCGSCCKFDEDDSEPVLKGVKLHYT.

In alternative embodiments, the vaccine comprises the S1 domain of the coronavirus strain COVID-19 surface glycoprotein, or an antigenic fragment thereof, where the S1 Domain stretches from Val16 until Arg685 of SEQ ID NO:2:

(SEQ ID NO: 3)
VNLTTRTQLPPAYTNSFTRGVYYPDKVFRSSVLHSTQDLFLPFFSNVTW
FHAIHVSGTNGTKRFDNPVLPFNDGVYFASTEKSNIIRGWIFGTTLDSK
TQSLLIVNNATNVVIKVCEFQFCNDPFLGVYYHKNNKSWMESEFRVYSS
ANNCTFEYVSQPFLMDLEGKQGNFKNLREFVFKNIDGYFKIYSKHTPIN
LVRDLPQGFSALEPLVDLPIGINITRFQTLLALHRSYLTPGDSSSGWTA
GAAAYYVGYLQPRTFLLKYNENGTITDAVDCALDPLSETKCTLKSFTVE
KGIYQTSNFRVQPTESIVRFPNITNLCPFGEVFNATRFASVYAWNRKRI
SNCVADYSVLYNSASFSTFKCYGVSPTKLNDLCFTNVYADSFVIRGDEV
RQIAPGQTGKIADYNYKLPDDFTGCVIAWNSNNLDSKVGGNYNYLYRLF
RKSNLKPFERDISTEIYQAGSTPCNGVEGFNCYFPLQSYGFQPTNGVGY
QPYRVVVLSFELLHAPATVCGPKKSTNLVKNKCVNFNFNGLTGTGVLTE
SNKKFLPFQQFGRDIADTTDAVRDPQTLEILDITPCSFGGVSVITPGTN
TSNQVAVLYQDVNCTEVPVAIHADQLTPTWRVYSTGSNVFQTRAGCLIG
AEHVNNSYECDIPIGAGICASYQTQTNSPRRAR.

In alternative embodiments, the vaccine comprises the RBD subdomain of the

Si domain of the coronavirus strain COVID-19 surface glycoprotein, or an antigenic fragment thereof, where the RBD subdomain stretches from Arg319 up to Phe541 of SEQ ID NO:2:

(SEQ ID NO: 4)
RVQPTESIVRFPNITNLCPFGEVFNATRFASVYAWNRKRISNCVADYSV
LYNSASFSTFKCYGVSPTKLNDLCFTNVYADSFVIRGDEVRQIAPGQTG
KIADYNYKLPDDFTGCVIAWNSNNLDSKVGGNYNYLYRLFRKSNLKPFE
RDISTEIYQAGSTPCNGVEGFNCYFPLQSYGFQPTNGVGYQPYRVVVLS
FELLHAPATVCGPKKSTNLVKNKCVNF.

In alternative embodiments, the vaccine comprises the variants of the coronavirus strain COVID-19 surface glycoprotein, or an antigenic fragment thereof, where the variants can be:

B.1.1.7, aka501Y.V1variant, 20I/501Y.V1, and UKCOVID variant;

P.1,akaB.1.1.28.1,20J/501Y.V3variant, and K417T/E484K/N501Y;

B.1.351,aka501.V2variant,20C/501Y.V2,and SouthAfricanCOVID-19variant;

B.1.427, and B.1.429aka20C/S:452R, USCaliforniaCOVID-19variants; and

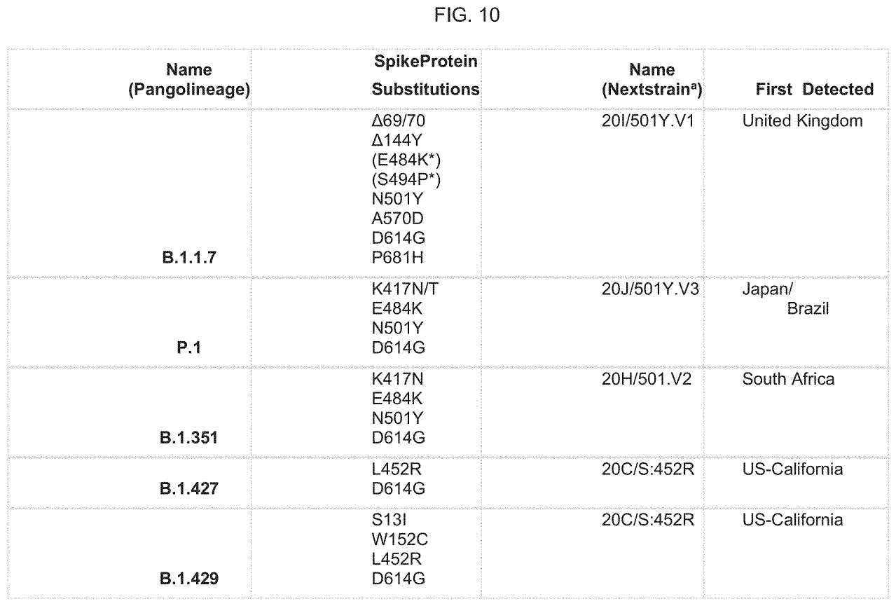

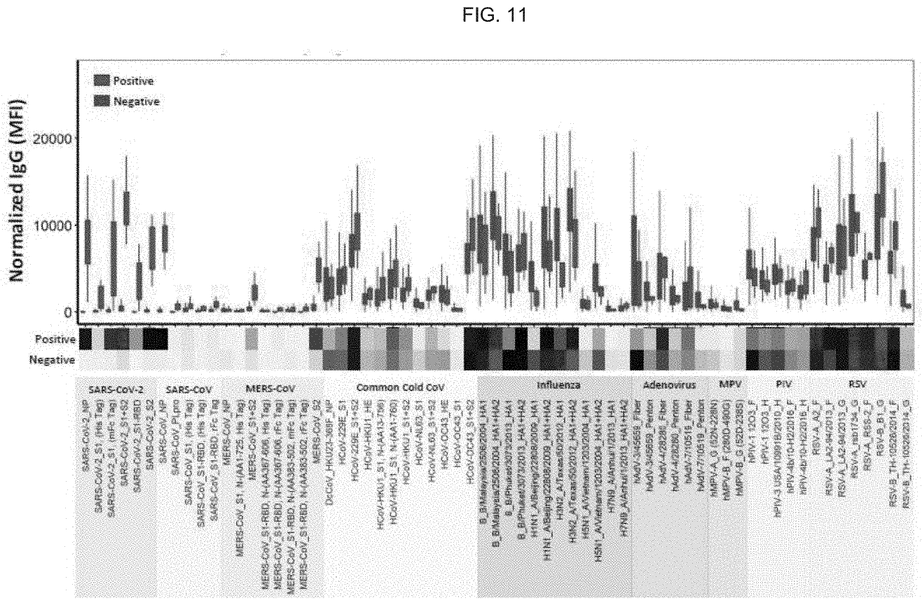

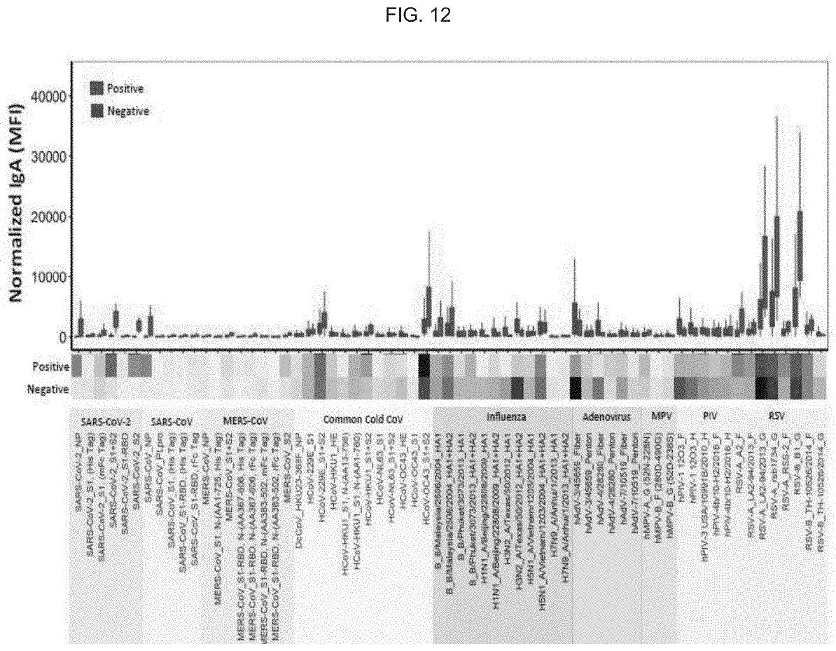

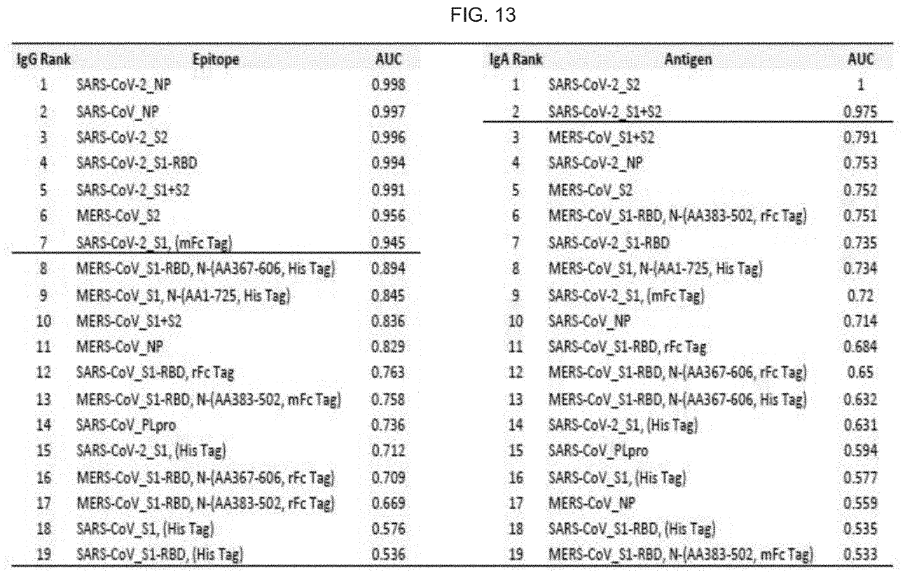

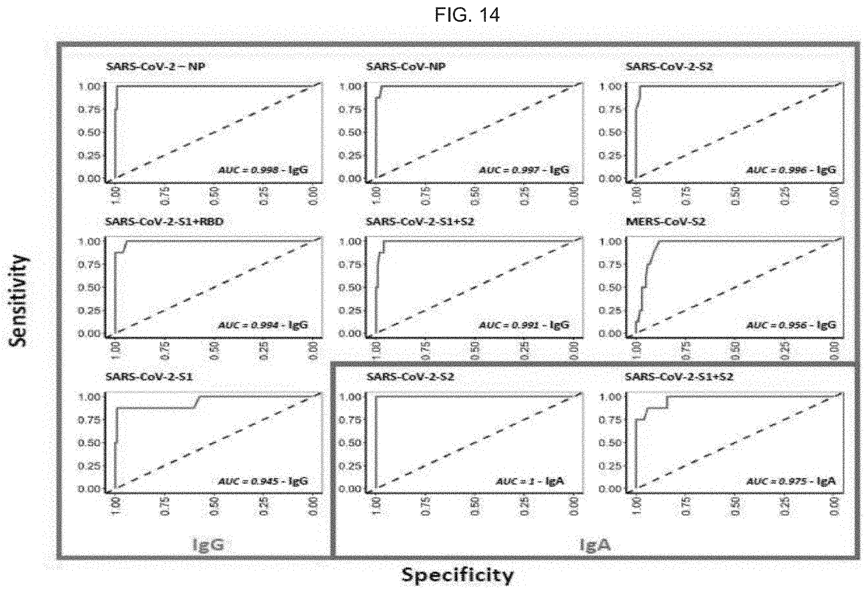

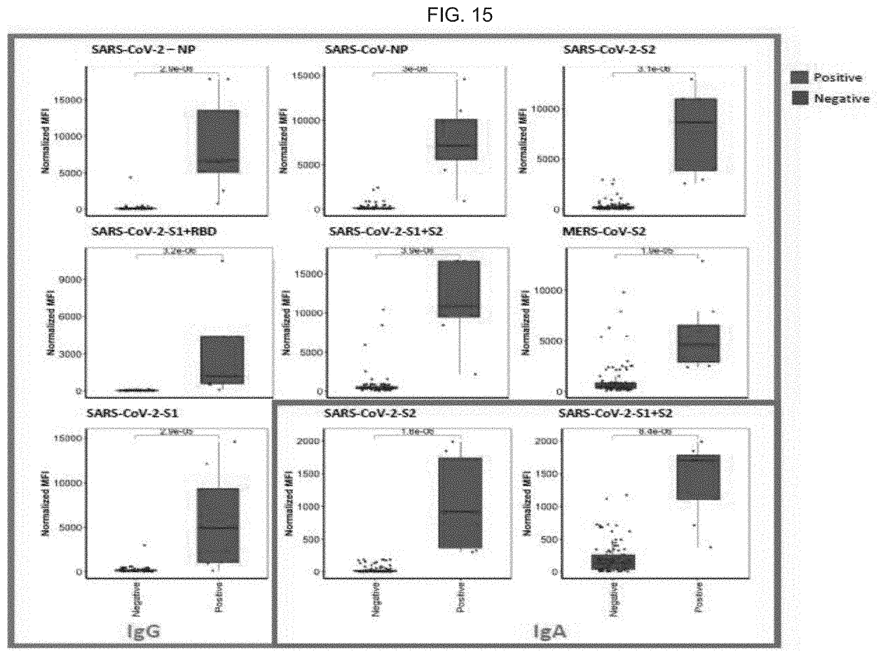

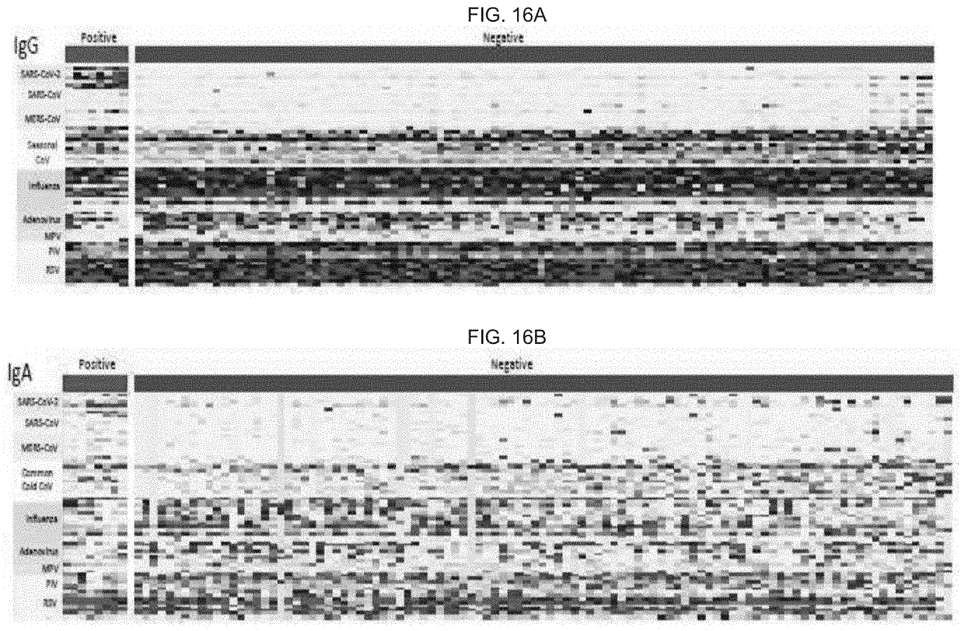

In alternative embodiments, peptides having mutations, for example, in the spike protein, for example as summarized in Table 4 (FIG. 10), are used in vaccines as provided herein and are administered in an individual in need thereof.

### Kits

Provided are kits for practicing methods as provided herein and/or comprising products of manufacture as provided herein; and optionally, the products of manufacture, including arrays, microarray, biochips, and vacccines, and kits can further comprise instructions for practicing methods as provided herein.

Any of the above aspects and embodiments can be combined with any other aspect or embodiment as disclosed here in the Summary, Figures and/or Detailed Description sections.

As used in this specification and the claims, the singular forms “a,” “an” and “the” include plural referents unless the context clearly dictates otherwise.

Unless specifically stated or obvious from context, as used herein, the term “or” is understood to be inclusive and covers both “or” and “and”.

Unless specifically stated or obvious from context, as used herein, the term “about” is understood as within a range of normal tolerance in the art, for example within 2 standard deviations of the mean. About can be understood as within 20%, 19%, 18%, 17%, 16%, 15%, 14%, 13%, 12% 11%, 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Unless specifically stated or obvious from context, as used herein, the terms “substantially all”, “substantially most of”, “substantially all of” or “majority of” encompass at least about 90%, 95%, 97%, 98%, 99% or 99.5%, or more of a referenced amount of a composition.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents. Incorporation by reference of these documents, standing alone, should not be construed as an assertion or admission that any portion of the contents of any document is considered to be essential material for satisfying any national or regional statutory disclosure requirement for patent applications. Notwithstanding, the right is reserved for relying upon any of such documents, where appropriate, for providing material deemed essential to the claimed subject matter by an examining authority or court.

Modifications may be made to the foregoing without departing from the basic aspects of the invention. Although the invention has been described in substantial detail with reference to one or more specific embodiments, those of ordinary skill in the art will recognize that changes may be made to the embodiments specifically disclosed in this application, and yet these modifications and improvements are within the scope and spirit of the invention. The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. Thus, the terms and expressions which have been employed are used as terms of description and not of limitation, equivalents of the features shown and described, or portions thereof, are not excluded, and it is recognized that various modifications are possible within the scope of the invention. Embodiments of the invention are set forth in the following claims.

The invention will be further described with reference to the examples described herein; however, it is to be understood that the invention is not limited to such examples.

### EXAMPLES

Unless stated otherwise in the Examples, all recombinant DNA techniques are carried out according to standard protocols, for example, as described in Sambrook et al. (2012) Molecular Cloning: A Laboratory Manual, 4th Edition, Cold Spring Harbor Laboratory Press, NY and in Volumes 1 and 2 of Ausubel et al. (1994) Current Protocols in Molecular Biology, Current Protocols, USA. Other references for standard molecular biology techniques include Sambrook and Russell (2001) Molecular Cloning: A Laboratory Manual, Third Edition, Cold Spring Harbor Laboratory Press, NY, Volumes I and II of Brown (1998) Molecular Biology LabFax, Second Edition, Academic Press (UK). Standard materials and methods for polymerase chain reactions can be found in Dieffenbach and Dveksler (1995) PCR Primer: A Laboratory Manual, Cold Spring Harbor Laboratory Press, and in McPherson at al. (2000) PCR - Basics: From Background to Bench, First Edition, Springer Verlag, Germany.

### Example 1: Exemplary Products of Manufacture

This example describes making and using exemplary embodiments as provided herein.

The current practice for diagnosis of SARS-CoV-2 infection relies on PCR testing of nasopharyngeal or respiratory specimens in a symptomatic patient at high epidemiologic risk. This testing strategy likely underestimates the true prevalence of infection, creating the need for serologic methods to detect infections missed by the limited testing to date. Here, we describe the development of a coronavirus antigen array containing immunologically significant antigens from SARS-CoV-2, in addition to SARS-CoV, MERS-CoV, common human coronavirus strains, and other common respiratory viruses. A preliminary study of human sera collected prior to the SARS-CoV-2 pandemic demonstrates overall high IgG reactivity to common human coronaviruses and low IgG reactivity to epidemic coronaviruses including SARS-CoV-2, with some cross-reactivity of conserved antigenic domains including S2 domain of spike protein and nucleocapsid protein. This array can be used to answer outstanding questions regarding SARS-CoV-2 infection, including whether baseline serology for other coronaviruses impacts disease course, and what antigens would be optimal for vaccine development.

Protein microarray methodology has been widely used to simultaneously perform binding assays against hundreds of antigens printed onto a nitrocellulose-coated slide for detection of multiple antibody isotypes⁵. This methodology was recently demonstrated for simultaneous measurement of IgG and IgA antibodies against over 250 antigens from diverse strains and subtypes of influenza⁶. This methodology has previously been applied to detect antibodies against the Si domains of SARS and MERS coronaviruses⁷.

### Methodology

### Specimen Collection

The blood specimens used in this study were collected for a larger study where residents of a college resident community in an Eastern university were monitored rospectively to identify acute respiratory infection (ARI) cases using questionnaires and RT-qPCR, so as to characterize contagious phenotypes including social connections, built environment, and immunologic phenotypes (citation). From among de-identified blood specimens for which future research use authorization was obtained, five specimens that showed high IgG reactivity against human coronaviruses in the larger study were chosen for validation of the coronavirus antigen microarray.

### Antigen Microarray

The coronavirus antigen microarray used in this investigation includes 67 antigens across subtypes expressed in either baculovirus or HEK-293 cells (see Tables 1-3). These antigens were printed onto microarrays, probed with human sera, and analyzed as previously described^{6.9.9}.

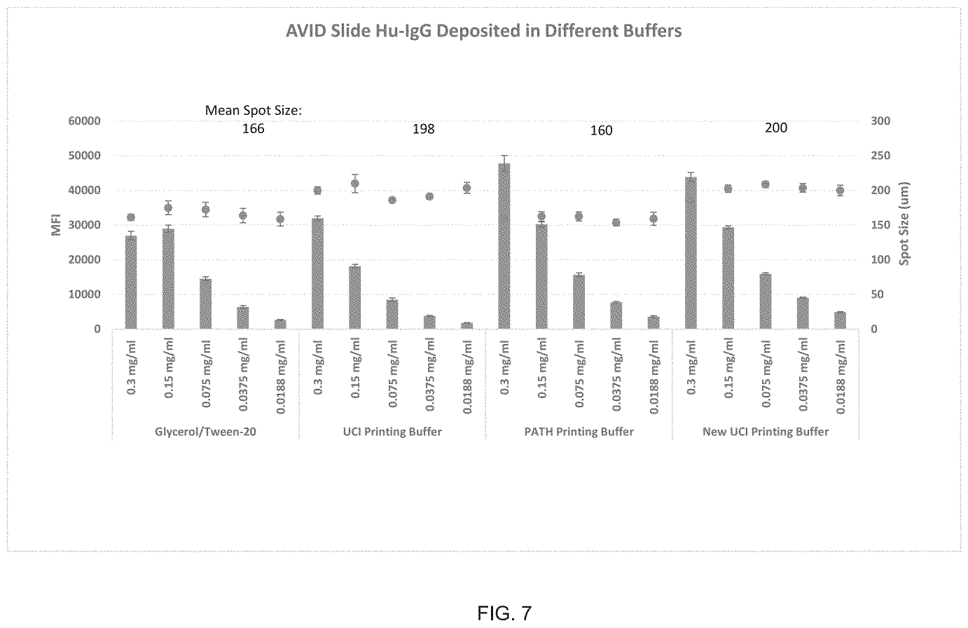

Briefly, lyophilized antigens were reconstituted to a concentration of 0.1 mg/mL in phosphate-buffered saline (PBS) with a printing buffer (comprising 0.15% Pluronic F127, 1% Trehalose, 1×PATH Printing buffer, see FIG. 7) and then printed onto nitrocellulose-coated slides from Grace Bio Labs (GBL, Bend, OR) using an OmniGrid 100 microarray printer (GeneMachines). The microarray slides were probed with human sera diluted 1:100 in 1×GVS Protein Array Blocking Buffer (GVS, Sanford, ME) overnight at 4° C., or for two hours (h) at room temperature (RT), washed with T-TBS buffer (20 mM Tris-HCl, 150 mM NaCl, 0.05% Tween-20 in ddH₂O adjusted to pH 7.5 and filtered) 3 times for 5 minutes each. Antibodies in sera were then detected with secondary antibodies to human IgG conjugated to an organic fluorescent dye (e.g., Alexa-Fluor) for 1 hour at room temperature, and then microarray slides were washed with T-TBS 3 times for 5 minutes each and dried. The slides were imaged using GenePix microarray scanner to measure background-subtracted median spot fluorescence. Mean fluorescence of the 4 replicate spots for each antigen was used for analysis.

### Statistical Analyses

Descriptive statistics were used to summarize the IgG reactivity as measured by mean fluorescence across antigen replicates. T-test or F-test were used to test for the mean differences in continuous variables across infection groups. All statistical analyses were conducted using R version 3.5.1 (R Foundation for Statistical Computing, Vienna, Austria).

### Results

Overall, the 5 sera tested on the coronavirus antigen microarray all showed high IgG seroreactivity to antigens from common human coronaviruses and other respiratory viruses with known seasonal circulation versus low IgG seroreactivity to antigens from epidemic viruses that were not circulating at time of collection (FIG. 1). Specifically, 4 of the 5 sera showed high IgG seroreactivity across the 4 common human coronaviruses, while all of the sera showed low IgG seroreactivity to SARS-CoV-2, SARS-CoV, and MERS-CoV. All 5 sera showed high IgG seroreactivity to RSV and parainfluenza viruses, while 3 of the 5 sera showed high IgG seroreactivity to adenoviruses. For influenza, all 5 sera showed high IgG seroreactivity to H1N1 and H3N2 influenza A and influenza B strains but low IgG seroreactivity to H5N1 and H7N9 influenza A strains.

With respect to specific antigens, the S1 domain of the spike protein including the receptor-binding domain (RBD) demonstrates very low cross-reactivity between epidemic coronaviruses and common human coronaviruses, whereas the S2 domain of the spike protein and the nucleocapsid protein (NP) are cross-reactive between these coronavirus subtypes. Similarly, the head domain of influenza hemagglutinin (HA1) demonstrates low cross-reactivity between seasonal and avian influenza strains, whereas the stalk domain (HA2) is cross-reactive between influenza virus subgroups, as seen between H1N1 and H5N1 influenza viruses.

### Discussion

This study yields several insights into cross-reactivity of common human coronavirus antibodies for SARS-CoV-2 antigens. The antibodies to the S1 and RBD domains of spike protein are highly subtype-specific, consistent with the high variability in these sequences between different human coronaviruses. Conversely, the S2 domain of spike protein and NP protein are more cross-reactive, consistent with these sequences being highly conserved across coronaviruses.

SARS-CoV-2 has caused a worldwide pandemic despite likely pre-existing cross-reactive antibodies to S2 domain and NP protein in most people, indicating that these antibodies are likely not protective, whereas antibodies to S1 and RBD domains are more likely to be protective. This observation favors a vaccination strategy based on S1 or RBD domains of spike protein over a vaccination strategy that also includes S2 domain or NP protein. In addition, S1 and RBD domains are more likely to generate subtype-specific serologic tests for population surveillance studies.

In addition, a key unexplained finding during the SARS-CoV-2 epidemic has been the low incidence of infection in children aged 15 and younger. This observation generates two related hypotheses: adults may have pre-existing antibodies against antigenically distinct coronaviruses that produce an ineffective humoral response to SARS-CoV-2 infection (antibody-dependent enhancement as demonstrated for dengue virus), or children younger than 15 may have initially encountered a coronavirus that is more closely related to SARS-CoV-2 so are more protected against this infection (immunologic imprinting or original antigenic sin as demonstrated for influenza virus). Both of these hypotheses would be informed by comparing the level of cross-reactive coronavirus antibodies in pediatric and adult cohorts and correlating these antibodies with incidence of severe disease.

### Conclusions

A coronavirus antigen microarray has been constructed with antigens from epidemic coronaviruses including SARS-CoV-2 and common human coronaviruses, in addition to other common respiratory viruses. A pilot study of 5 naive human sera shows high IgG seroreactivity to common human coronaviruses but low IgG seroreactivity to SARS-CoV-2, with some cross-reactivity seen for S2 domain of spike protein and nucleocapsid protein. Further studies are needed including with SARS-CoV-2 convalescent sera to fully realize the potential of this novel methodology to characterize the seroprevalence of SARS-CoV-2 and the impact of pre-existing cross-reactive antibodies on the disease course.

### Example 2: Exemplary Products of Manufacture

This example describes making and using exemplary embodiments as provided herein.

The current practice for diagnosis of COVID-19, based on SARS-CoV-2 PCR testing of pharyngeal or respiratory specimens in a symptomatic patient at high epidemiologic risk, likely underestimates the true prevalence of infection. Serologic methods can more accurately estimate the disease burden by detecting infections missed by the limited testing performed to date.

Here, we describe the validation of an exemplary coronavirus antigen microarray as provided herein containing immunologically significant antigens from SARS-CoV-2, in addition to SARS-CoV, MERS-CoV, common human coronavirus strains, and other common respiratory viruses. A comparison of antibody profiles detected on the array from control sera collected prior to the SARS-CoV-2 pandemic versus convalescent blood specimens from virologically confirmed COVID-19 cases demonstrates near complete discrimination of these two groups, with improved performance from use of antigen combinations that include both spike protein and nucleoprotein. This exemplary array can be used as a diagnostic tool, as an epidemiologic tool to more accurately estimate the disease burden of COVID-19, and as a research tool to correlate antibody responses with clinical outcomes.

COVID-19 caused by the SARS-CoV-2 virus is a worldwide pandemic with 30 significant morbidity and mortality estimates from 1-4% of confirmed cases. The current case definition for confirmed SARS-CoV-2 infection relies on PCR-positive pharyngeal or respiratory specimens, with testing largely determined by presence of fever or respiratory symptoms in an individual at high epidemiologic risk. However, this case definition likely underestimates true prevalence, as individuals who develop subclinical infection that does not produce fever or respiratory symptoms are unlikely to be tested, and testing by PCR of pharyngeal or respiratory specimens is only around 60-80% sensitive depending on sampling location and technique and the patient's viral load2. Widespread testing within the United States is also severely limited by the lack of available testing kits and testing capacity limitations of available public and private laboratories. Therefore, the true prevalence of SARS-CoV-2 infection is likely much higher than currently reported case numbers would indicate.

Serology can play an important role in defining the true prevalence of COVID-19, particularly for subclinical infection2. Early studies of serology demonstrate high sensitivity to detect confirmed SARS-CoV-2 infection, with antibodies to virus detected approximately 1 to 2 weeks after symptom onset³. Unlike PCR positivity, SARS-CoV-2 antibodies are detectable throughout the disease course and persist indefinitely⁴. Multiple serologic tests have been developed for COVID-19⁵ including a recently FDA-approved lateral flow assay. However, these tests are limited to detection of antibodies against one or two antigens, and cross-reactivity with antibodies to other human coronaviruses that are present in all adults⁶ is currently unknown. Prior use of serology for detection of emerging coronaviruses focused on antibodies against the spike (S) protein, particularly the 51 domain, and the nucleocapsid protein (NP)⁷. However, the optimal set of antigens to detect strain-specific coronavirus antibodies remains unknown.

Protein microarray technology can be used to detect antibodies of multiple isotypes against hundreds of antigens in a high throughput manner^{8,9} so is well suited to serologic surveillance studies. This technology, which has previously been applied to other emerging coronaviruses¹⁰, is based on detection of binding antibodies, which are well-correlated with neutralizing antibodies¹¹ but do not require viral culture in biosafety level 3 facilities.

Described herein and provided is an exemplary coronavirus antigen microarray (CoVAM) that includes antigens from SARS-CoV-2, which we tested it on human sera collected prior to the pandemic to demonstrate low cross-reactivity with antibodies from human coronaviruses that cause the common cold, particularly for the Si domain2. Here, we further validate this methodology using convalescent blood specimens from COVID-19 cases confirmed by positive SARS-CoV-2 PCR.

### Methodology

### Specimen Collection

A total of 22 de-identified SARS-CoV-2 convalescent blood specimens were collected from nasopharyngeal PCR-positive individuals from different sources with associated data on symptom onset, positive PCR test, and collection. Two sera were obtained as de-identified discarded laboratory specimens from acute COVID-19 patients from the Oregon Health Sciences University Hospital (OHSU), Portland, OR. These were sourced from discarded clinical laboratory specimens exempted from informed consent and IRB approval under condition of patient anonymity. An additional two sera were obtained from recovered COVID patients at Vitalant Research Institute in San Francisco, CA under an IRB approved protocol. One convalescent plasma was obtained by Cerus Corporation after isolation from a large-volume apheresis collection following standard protocol from a documented recovered COVID-19 blood donor who was more than 28 days post symptomatic. Four plasma samples were obtained from outpatients of the University Hospital Basel, University of Basel, Basel, Switzerland. These patients were screened in accordance with Swiss regulations on blood donation and approved as plasma donors according to the Blood Transfusion Service of the Swiss Red Cross with informed consent. These donors were diagnosed with COVID-19 based on SARS-CoV-2 positive nasopharyngeal swab PCR tests. At time of plasma donation, each had two negative nasopharyngeal swab SARS-CoV-2 PCR tests and negative SARS-CoV-2 PCR tests in blood, and they were qualified as plasma donors. Plasma was collected from these convalescent donors at the Regional Blood Transfusion Service of the Swiss Red Cross in accordance with national regulations.

A total of 144 de-identified pre-pandemic control sera used in this study were collected between November 2018 and May 2019 for a larger study where residents of a college resident community in the Eastern United States were monitored prospectively to identify acute respiratory infection (ARI) cases using questionnaires and RT-qPCR, so as to characterize contagious phenotypes including social connections, built environment, and immunologic phenotypes¹². Electronic informed consents including future research use authorization was obtained under protocols approved by the Institutional Review Boards (IRBs) of the University of Maryland and the Department of Navy Human Research Protections Office.

### Specimen Testing on Coronavirus Antigen Microarray

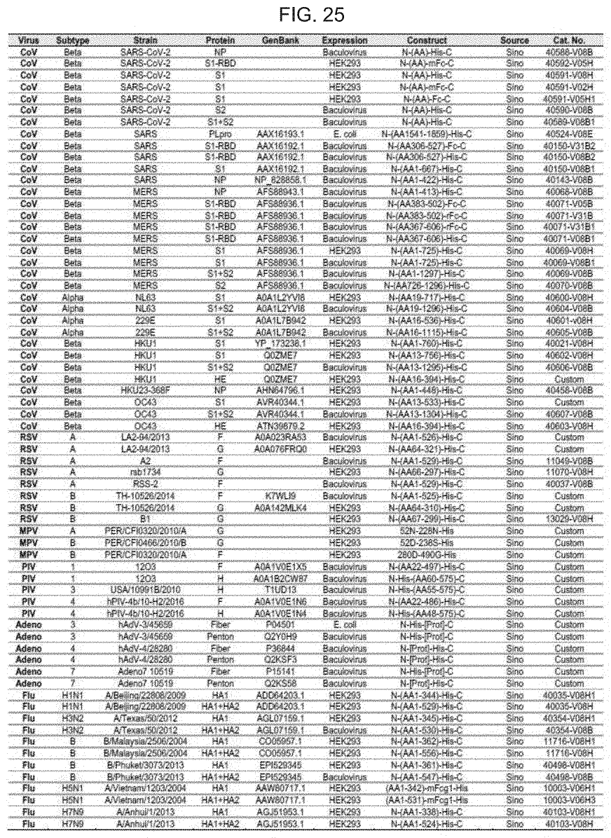

The coronavirus antigen microarray used in this investigation includes 67 antigens across subtypes expressed in either baculovirus or HEK-293 cells (see the table of FIG. 25). These antigens were provided by Sino Biological U.S. Inc. (Wayne, PA) as either catalog products or custom synthesis service products. The antigens were printed onto microarrays, probed with human sera, and analyzed as previously described^{9,13,14.}

Briefly, lyophilized antigens were reconstituted with sterile water to a concentration of 0.1 mg/mL bringing protein solution to lx phosphate-buffered saline (PBS) and printing buffer was added. Antigens were then printed onto ONCYTE AVID™ nitrocellulose-coated slides (Grace Bio-Labs, Bend, OR) using an OMNIGRID™ 100 microarray printer (GENEMACHINES™). The microarray slides were probed with human sera diluted 1:100 in 1×Protein Array Blocking Buffer (GVS Life Sciences, Sanford, ME) overnight at 4° C. and washed with T-TBS buffer (20 mM Tris-HCl, 150 mM NaCl, 0.05% Tween-20 in ddH2O adjusted to pH 7.5 and filtered) 3 times for 5 minutes each. A mixture of human IgG and IgA secondary antibodies conjugated to quantum dot fluorophores Q800 and Q585 respectively was applied to each of the microarray pads and incubated for 2 hours at room temperature, and pads were then washed with T-TBS 3 times for 5 minutes each and dried. The slides were imaged using ARRAYCAM™ imager (Grace Bio-Labs, Bend, OR) to measure background-subtracted median spot fluorescence. Non-specific binding of secondary antibodies was subtracted using saline control. Mean fluorescence of the 4 replicate spots for each antigen was used for analysis.

### Statistical Analyses

The mean fluorescence intensity (MFI) of each antigen was determined by the average of the median fluorescence signal of four replicate spots. The fluorescence signal for each spot was determined by its signal intensity subtracted by the background fluorescence. Antigens containing a human Fc tag were removed from the analysis, as the secondary antibodies used for quantification are known to bind to human Fc; non-human Fc tag did not interfere with the assay. All statistical analyses were conducted using R version 3.6.3 (R Foundation for Statistical Computing, Vienna, Austria).

MFI was normalized by the quantile normalization method using the proprocessCore package (version 1.48.0). As a target for normalization, a vector containing the median MFI for IgG or IgA was constructed. Descriptive statistics were used to summarize the IgA and IgG reactivity measured as MFI. Wilcoxon Rank Sum tests with p<0.05 corrected for multiple comparisons were used to compare the mean differences between groups. In order to rank the antigens from SARS-CoV-2, SARS-CoV, and MERS-CoV for performance in discriminating the positive and negative groups, the Receiver Operating Characteristic Area Under the Curve (ROC AUC) values for each antigen were calculated by comparing positive and negative specimens using the pROC package (version 1.16.2). For this, the samples were randomly partitioned into two groups, at a ratio of 75%/25%, using the caret package (version 6.9-86). The group with 75% of the samples was used to create a regression model using the glm function form the stats package (version 3.6.3). The 25% subset was used to predict the outcome of each sample being classified as negative or positive using the stat package and the AUC value calculated. This process was repeated for one thousand times and the final AUC values calculated as the median values of all repetitions.

Next, in order to evaluate the benefit of combining antigens for an increased prediction performance, the top ranked antigens, using a cutoff point of auc=0.85, were combined into groups of all possible combinations from 2 to 4 antigens using the combinat package (version 0.0-8). Again, the auc values for each combination were calculated using the same procedure as for individual antigens and the calculated AUCs are representative of the median AUC from one thousand repetitions.

The optimal sensitivity and specificity for each antigen and combination of antigens was calculated based on the maximum Youden Index. Data visualization was performed using the ggplot2 package (version 3.3.0) or pROC package.

### Results

### Construction of Coronavirus Antigen Microarray

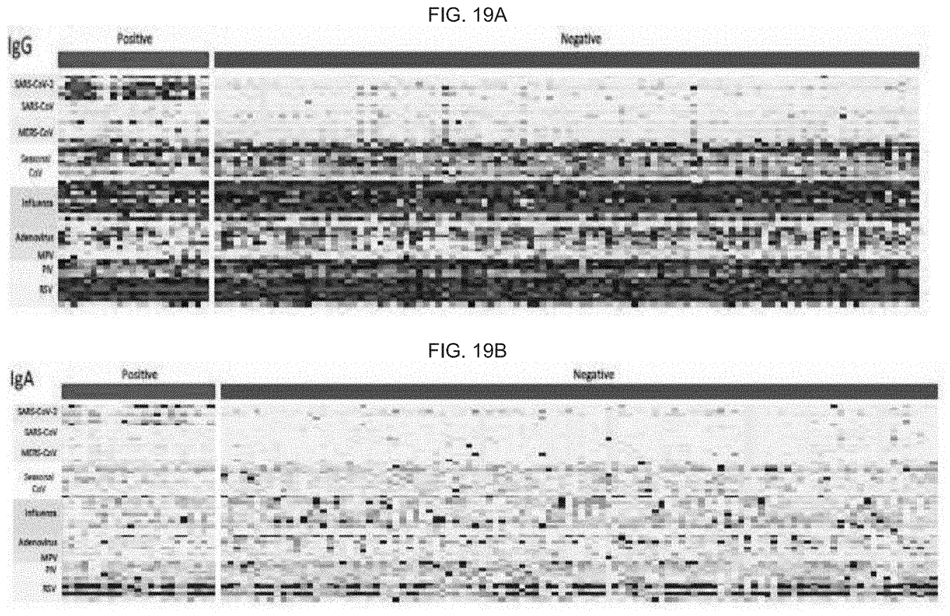

A coronavirus antigen microarray (COVAM) was constructed containing 65 antigens that are causes of acute respiratory infections. The array was used to detect IgG and IgA antibodies present in a collection of blood specimens from recovered COVID-19 patients and pre-pandemic control sera, and the results are shown on the heatmap in FIG. 19. The viral antigens printed on this array are from epidemic coronaviruses including SARS-CoV-2, SARS-CoV, and MERS-CoV, common cold coronaviruses (HKU1, OC43, NL63, 229E), and multiple subtypes of influenza, adenovirus, metapneumovirus, parainfluenza, and respiratory syncytial virus as listed in the table of FIG. 25. The SARS-CoV-2 antigens on this array include spike protein (S), the receptor-binding (RBD), S1, and S2 domains the whole protein (S1+S2), and the nucleocapsid protein (NP). There is a similar set of antigens represented on the array from SARS-CoV, MERS-CoV, and the four common cold coronaviruses.

### Discrimination of SARS-CoV-2 Convalescent Blood Specimens Using Coronavirus Antigen Microarray

To determine the antibody profile of SARS-CoV-2 infection, the differential reactivity to these antigens was evaluated for SARS-CoV-2 convalescent blood specimens from PCR-positive individuals (positive group) and sera collected prior to the COVID-19 pandemic from naive individuals (negative control group). As shown in the heatmap (FIG. 19), the positive group is highly reactive against SARS-CoV-2 antigens. This is more evident for the IgG reactivity then for IgA. The negative controls do not show high reactivity to SARS-CoV-2, SARS-CoV or MERS-CoV antigens despite showing high reactivity to the common cold coronavirus antigens.

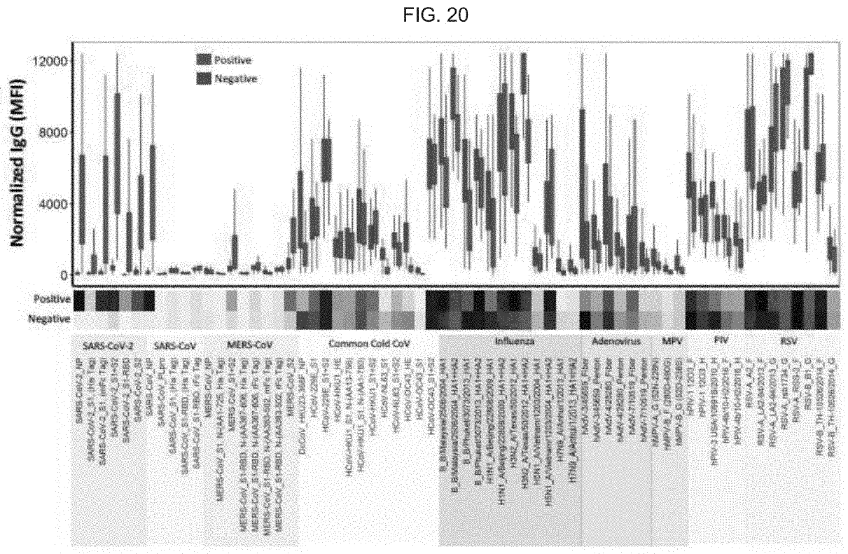

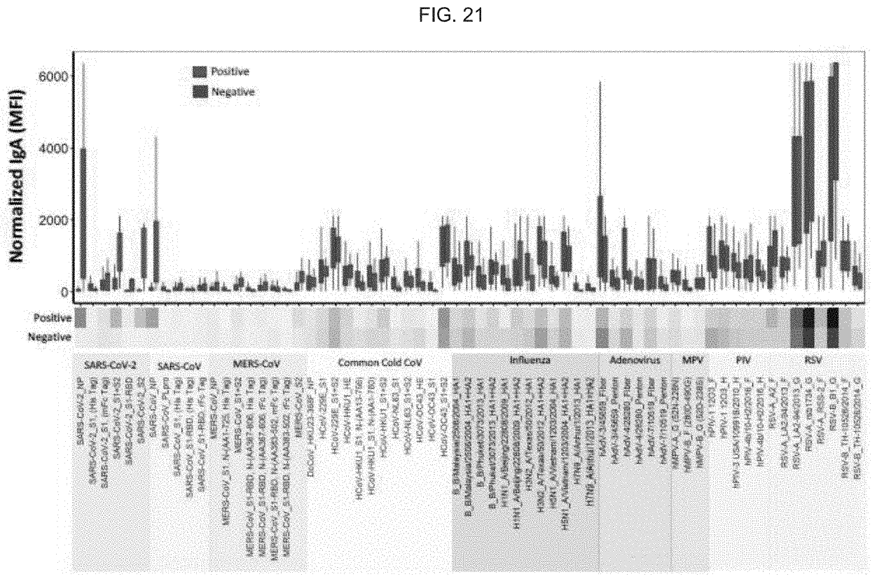

With respect to specific antigens, positive group displays high IgG reactivity to SARS-CoV-2 NP, S2, and S1+S2 antigens and to a lesser degree SARS-CoV-2 Si (FIG. 20). The positive group also demonstrates high IgG cross-reactivity against SARS-CoV NP and MERS-CoV S2 and S1+S2 antigens, while the negative group demonstrates low cross-reactivity with S1+S2 and S2 antigens from SARS-CoV-2 and MERS-CoV and no cross-reactivity against other SARS-CoV-2 antigens. The IgA reactivity profile is shown on FIG. 21. Overall, IgA seems to follow a similar pattern to IgG, with higher reactivity to SARS-CoV-2 NP, S2 and S1+S2, cross-reactivity to SARS-CoV NP, but no cross-reactivity to the MERS-CoV antigens.

The two groups do not differ significantly in reactivity to antigens from common cold coronaviruses or other respiratory viruses for either IgG or IgA. The differences between the groups appear to be restricted to SARS-CoV-2 antigens and cross-reactive SARS-CoV and MERS-CoV antigens, so these antigens from epidemic coronaviruses were the focus of subsequent analysis.

### \Selection of High-Performing Antigens to Detect SARS-CoV-2 Infection

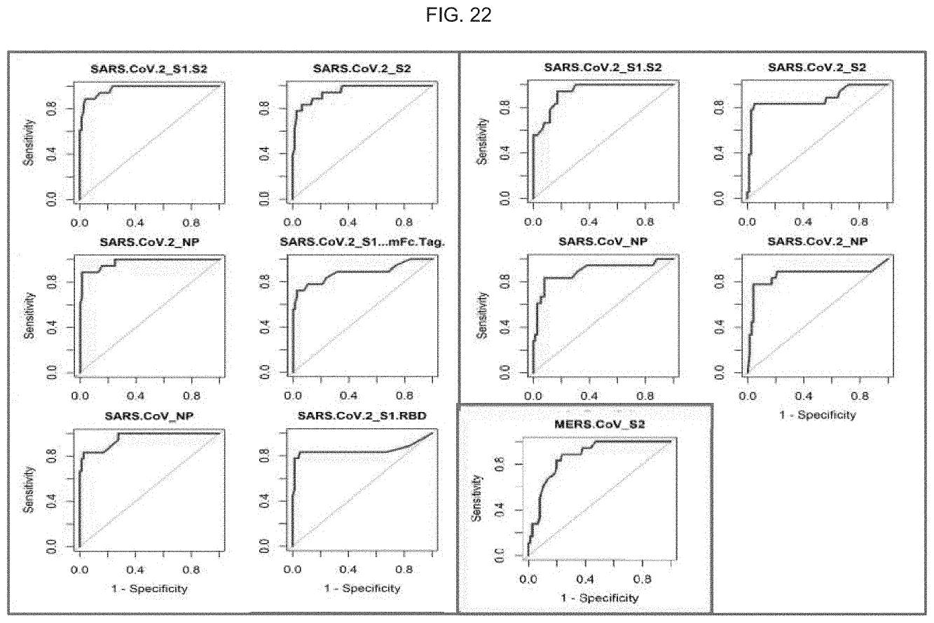

The sensitivity a specificity of each antigen from SARS-CoV-2, SARS-CoV, and MERS-CoV was evaluated to discriminate the positive group from the negative group across a full range of assay cutoff values using Receiver Operating Characteristic (ROC) curves for which Area Under Curve (AUC) was measured (FIG. 22). High-performing antigens for detection of IgG or IgA were defined by ROC AUC≥0.85.

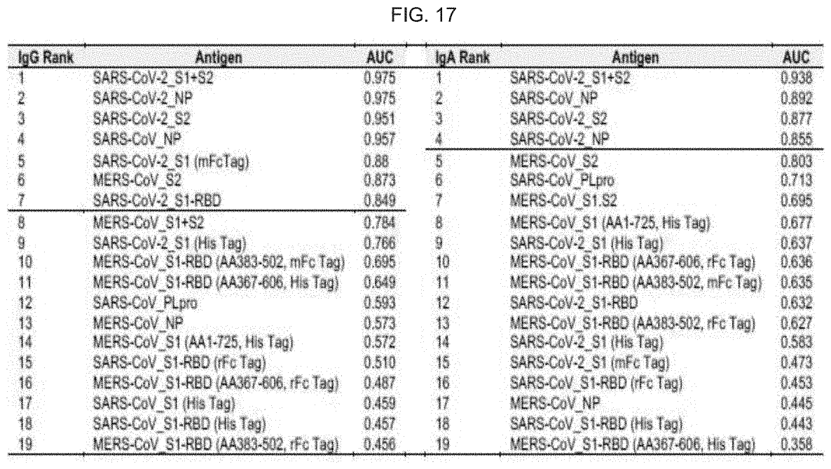

Although the antigen ranking was different for IgG and IgA, most of the high-ranking antigens were from SARS-CoV-2 and most of the low-ranking antigens were from SARS-CoV and MERS-CoV (see table of FIG. 17). Among the high-performing antigens, four antigenswere ranked as high-performing antigens for both IgG and IgA: SARS-CoV-2 NP, SARS-CoV NP, SARS-CoV-2 S1+S2, and SARS-CoV-2 S2. For IgG, additional high-performing antigens included SARS-CoV-2 Si (with mouse Fc tag) and RBD and MERS-CoV S2.

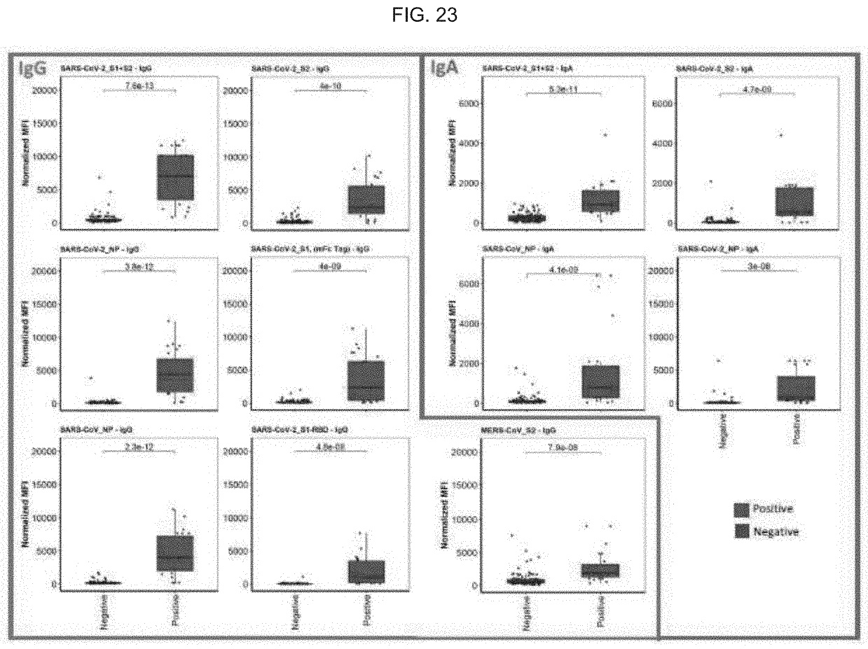

Each of the high-performing antigens discriminated between the positive group and the negative group with high significance based on differential reactivity as shown in FIG. 23. Positive samples consistently show significantly higher reactivity to these antigens than the negative controls (p<10⁻⁷). For IgG, the median reactivity to the top antigens for the positive group is 20-fold higher than for the negative group, while a 6-fold difference is observed for IgA.

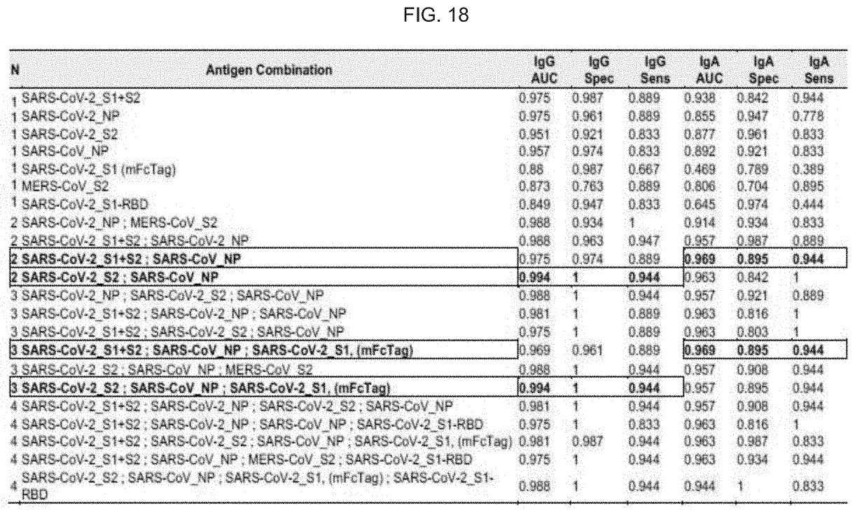

The optimal sensitivity and specificity were also estimated for the six high-performing antigens based on the Youden Index (see table of FIG. 18). For IgG, the lowest sensitivity was seen for SARS-CoV-2 1, which correlates with the relatively lower reactivity to this antigen in the positive group, while sensitivity was high for the other antigens. The lowest specificity was seen for SARS-CoV-2 S2, which correlates with the cross-reactivity for this antigen seen in a subset of the negative group, while specificity was high for the other antigens. Conversely, for IgA, the highest sensitivity is seen with SARS-CoV-2 S1+S2, and the highest specificity is seen with SARS-CoV-2 S2.

### Determination of Optimal Antigen Combination to Detect SARS-CoV-2 Infection

In order to estimate the gain in performance by combining antigens, all possible combinations of up to 4 of the 7 high-performing antigens were tested in silico for performance in discriminating the positive and negative groups. The ROC curve with AUC, sensitivity, and specificity was calculated for each combination. For both IgG and IgA, there is a clear gain in performance by combining antigens.

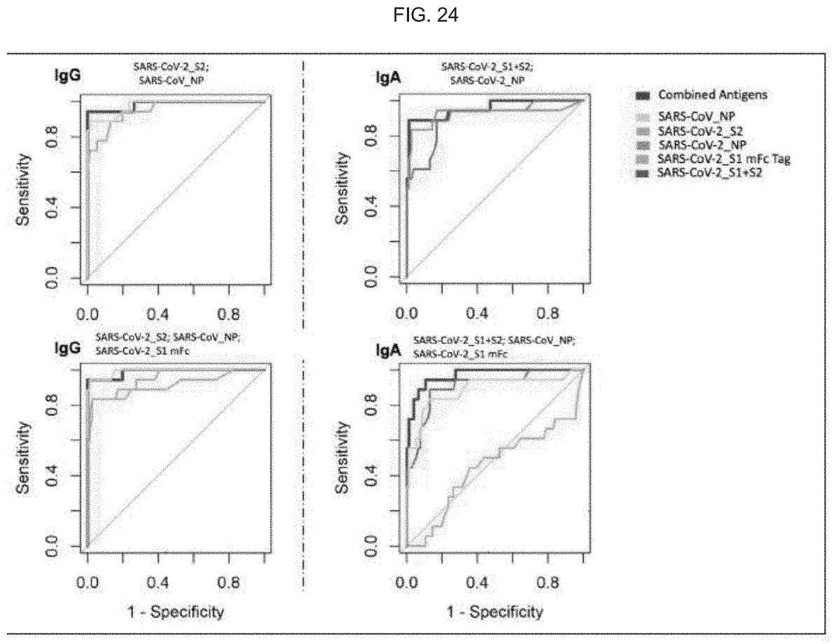

The highest performing antigen combinations for each number of antigens are summarized in the table of FIG. 18, and ROC curves for the top-performing antigen combinations overall for IgG and IgA with comparison to each individual antigen are shown in FIG. 24. For IgG, the best discrimination was achieved with the 2-antigen combination of SARS-CoV-2 S2 and SARS-CoV NP, with similar performance upon the addition of SARS-CoV- 2 Si with mouse Fc tag (AUC=0.994, specificity=1, sensitivity=0.944). For IgA, the top performance was achieved with the 2-antigen combination of SARS-CoV-2 S1+S2 and SARS-CoV NP, with similar performance upon the addition of SARS-CoV-2 S1 with mouse Fc tag (AUC=0.969, specificity=0.895, sensitivity =0.944). The addition of a fourth antigen decreased the performance for both IgG and IgA.

The table of FIG. 17 shows the receiver operating characteristic area under curve (ROC AUC) for SARS-CoV-2, SARS-CoV, and MERS-CoV antigens. ROC AUC values for discrimination of positive and negative sera were derived for each individual antigen for both IgG and IgA and ranked, and high-performing antigens with ROC AUC≥0.86 are indicated above the lines.

FIG. 19A-B illustrates a heatmap for the exemplary coronavirus antigen microarray: the heatmap shows IgG (FIG. 19A) and IgA (FIG. 19B) reactivity measured as mean fluorescence intensity across four replicates, against each antigen organized into rows color coded by virus, for sera organized into columns classified as positive (convalescent from PCR-positive individuals) or negative (prior to pandemic from naïve individuals). Reactivity is represented by color (white=low, black=mid, red=high).

### Discussion

This study reveals several insights into the antibody response to SARS-CoV-2 infection. The antibody profiles of naïve individuals include high IgG reactivity to common cold coronaviruses with low-level cross-reactivity with S2 domains from SARS-CoV-2 and other epidemic coronaviruses, which is not surprising given the high degree of sequence homology and previously observed serologic cross-reactivity¹⁵ between S2 domains of betacoronaviruses, a group that includes SARS-CoV-2, SARS-CoV, MERS, and common cold coronaviruses HKU1 and OC43. This low-level cross-reactivity occurs in approximately 7% of unexposed individuals (FIG. 19), which leads to hypotheses regarding whether these individuals differ in COVID-19 susceptibility and outcomes. However, naive individuals do not show cross-reactivity to other SARS-CoV-2 antigens. Even for the nucleocapsid protein, which also has high sequence homology between betacoronaviruses, cross-reactivity is only seen between SARS-CoV-2 and SARS-CoV and not with MERS-CoV or common cold coronaviruses.

In addition, the quantitative difference between high antibody reactivity to SARS-CoV-2 S2 in the positive group and low-level antibody cross-reactivity in the negative group is large enough that these antigens still discriminate these groups with high significance.

This study also informs antigen selection and design for population surveillance and clinical diagnostic assays and vaccine development. The optimal assay to discriminate SARS-CoV-2 convalescent sera from pre-pandemic sera is a combination of 2 antigens that includes S2 and NP. As an individual antigen, the S2 demonstrates cross-reactivity with negative control sera which leads to low specificity, but this antigen adds predictive power when combined with the more specific NP antigen. The observation that unexposed individuals with antibodies to common cold coronaviruses do not show cross-reactivity to SARS-CoV-2 NP dispels concerns that the high sequence homology of this protein across betacoronaviruses would impair its performance as a diagnostic antigen. The low-level antibody cross-reactivity of a subset of unexposed ndividuals for SARS- CoV-2 spike protein containing S2 domain may not preclude its use as a diagnostic antigen given large quantitative difference in antibody reactivity between positive and negative groups, but this cross-reactivity may influence response to vaccination with spike protein antigens containing the S2 domain in this subset of individuals.

The coronavirus antigen microarray can be useful both as an epidemiologic tool and as a research tool. The high throughput detection of SARS-CoV-2-specific antibody profiles that reliably distinguish COVID-19 cases from negative controls can be applied to large-scale population surveillance studies for a more accurate estimation of the true prevalence of disease than can be achieved with symptom-based PCR testing. In addition, detection of these antibodies in SARS-CoV-2 convalescent plasma donations can provide validation prior to clinical use for passive immunization. The variation in the SARS-CoV-2 antibody profiles among acute and convalescent donors suggests that epitope characterization of convalescent donor plasma will be informative for evaluation of passive immune therapy efficacy in COVID-19 patients. The central role of inflammation in the pathogenesis of severe COVID-19¹⁶ can be more closely studied by analyzing both strain-specific and cross-reactive antibody responses, particularly to test hypotheses regarding antibody-dependent enhancement with critical implications for vaccine development¹⁷.

Data provided herein demonstrates that an exemplary coronavirus antigen microarray as provided herein containing a panel of antigens from SARS-CoV-2 in addition to other human coronaviruses was able to reliably distinguish convalescent plasma of PCR-positive COVID-19 cases from negative control sera collected prior to the pandemic. Antigen combinations including both spike protein and nucleoprotein demonstrated improved performance compared to each individual antigen. Further studies are needed to apply this methodology to large-scale serologic surveillance studies and to correlate specific antibody responses with clinical outcomes.

### REFERENCES EXAMPLE 1

1. Leung, N. H., et al. Review Article: The Fraction of Influenza Virus Infections That Are Asymptomatic: A Systematic Review and Meta-analysis. Epidemiology 26, 862-872, doi:10.1097/EDE.0000000000000340 (2015).
2 Zhou, W., et al. First infection by all four non-severe acute respiratory syndrome human coronaviruses takes place during childhood. BMC Infect Dis 13, 433, doi:10.1186/1471-2334-13-433 (2013).
3 Agnihothram, S. et al. Evaluation of serologic and antigenic relationships between middle eastern respiratory syndrome coronavirus and other coronaviruses to develop vaccine platforms for the rapid response to emerging coronaviruses. J Infect Dis 209, 995-1006, doi:10.1093/infdis/jit609 (2014).

4 Chan, C. M. et al. Examination of seroprevalence of coronavirus HKU1 infection with S protein-based ELISA and neutralization assay against viral spike pseudotyped virus. J Clin Virol 45, 54-60, doi:10.1016/j.jcv.2009.02.011 (2009).
5 Davies, D. H. et al. Profiling the humoral immune response to infection by using proteome microarrays: high-throughput vaccine and diagnostic antigen discovery. Proc Natl Acad Sci USA 102, 547-552, doi:10.1073/pnas.0408782102 (2005).
6 Khan, S. et al. Use of an Influenza Antigen Microarray to Measure the Breadth of Serum Antibodies Across Virus Subtypes. J Vis Exp, doi:10.3791/59973 (2019).
7 Reusken, C. et al. Specific serology for emerging human coronaviruses by protein microarray. Euro Surveill 18, 20441, doi:10.2807/1560-7917.es2013.18.14.20441 (2013).
8 Jain, A. et al. Evaluation of quantum dot immunofluorescence and a digital CMOS imaging system as an alternative to conventional organic fluorescence dyes and laser scanning for quantifying protein microarrays. Proteomics 16, 1271-1279, doi:10.1002/pmic.201500375 (2016).
9 Nakajima, R. et al. Protein Microarray Analysis of the Specificity and Cross-Reactivity of Influenza Virus Hemagglutinin-Specific Antibodies. mSphere 3, doi:10.1128/mSphere.00592-18 (2018).

### REFERENCES EXAMPLE 2

1 Cao, Y., et al. Imaging and Clinical Features of Patients With 2019 Novel Coronavirus SARS-CoV-2: A systematic review and meta-analysis. J Med Virol, doi:10.1002/jmv.25822 (2020).
2 Tang, Y. W., et al. The Laboratory Diagnosis of COVID-19 Infection: Current Issues and Challenges. J Clin Microbiol, doi:10.1128/JCM.00512-20 (2020).
3 Zhao, J. et al. Antibody responses to SARS-CoV-2 in patients of novel 25 coronavirus disease 2019. Clin Infect Dis, doi:10.1093/cid/ciaa344 (2020).
4 To, K. K. et al. Temporal profiles of viral load in posterior oropharyngeal saliva samples and serum antibody responses during infection by SARS-CoV-2: an observational cohort study. Lancet Infect Dis, doi:10.1016/S1473-3099(20)30196-1 (2020).
5 Liu, W. et al. Evaluation of Nucleocapsid and Spike Protein-based ELISAs for detecting antibodies against SARS-CoV-2. J Clin Microbiol, doi:10.1128/JCM.00461-20 (2020).
6 Zhou, W., Wang, W., Wang, H., Lu, R. & Tan, W. First infection by all four non-severe acute respiratory syndrome human coronaviruses takes place during childhood. BMC Infect Dis 13, 433, doi:10.1186/1471-2334-13-433 (2013).
7 Agnihothram, S. et al. Evaluation of serologic and antigenic relationships between middle eastern respiratory syndrome coronavirus and other coronaviruses to develop vaccine platforms for the rapid response to emerging coronaviruses. J Infect Dis 209, 995-1006, doi:10.1093/infdis/jit609 (2014).
8 Davies, D. H. et al. Profiling the humoral immune response to infection by using proteome microarrays: high-throughput vaccine and diagnostic antigen discovery. Proc Natl Acad Sci USA 102, 547-552, doi:10.1073/pnas.0408782102 (2005).
9 Khan, S. et al. Use of an Influenza Antigen Microarray to Measure the Breadth of Serum Antibodies Across Virus Subtypes. J Vis Exp, doi:10.3791/59973 (2019).
10 Reusken, C. et al. Specific serology for emerging human coronaviruses by protein microarray.Euro Surveill 18, 20441, doi:10.2807/1560-7917.es2013.18.14.20441 (2013).
11 Chan, C. M. et al. Examination of seroprevalence of coronavirus HKU1 infection with S protein-based ELISA and neutralization assay against viral spike pseudotyped virus. J Clin Virol 45, 54-60, doi:10.1016/j.jcv.2009.02.011 (2009).
12 Zhu, S. et al. Ventilation and laboratory confirmed acute respiratory infection (ARI) rates in college residence halls in College Park, Maryland. Environ Int 137, 105537, doi:10.1016/j.envint.2020.105537 (2020).
13 Jain, A. et al. Evaluation of quantum dot immunofluorescence and a digital CMOS imaging system as an alternative to conventional organic fluorescence dyes and laser scanning for quantifying protein microarrays. Proteomics 16, 1271-1279, doi:10.1002/pmic.201500375 (2016).
14 Nakajima, R. et al. Protein Microarray Analysis of the Specificity and Cross-Reactivity of Influenza Virus Hemagglutinin-Specific Antibodies. mSphere 3, doi:10.1128/mSphere.00592-18 (2018).

A number of embodiments of the invention have been described. Nevertheless, it can be understood that various modifications may be made without departing from the spirit and scope of the invention. Accordingly, other embodiments are within the scope of the following claims.

1. A product of manufacture for detecting the presence of a coronavirus in a sample, or to detect a coronavirus infection, wherein optionally the coronavirus is the strain COVID-19,
wherein affixed or attached (optionally covalently affixed or attached) onto a surface of the product of manufacture is:
(a) at least one peptide or polypeptide epitope or antigen derived from a COVID-19 coronovirus or variant thereof, or a plurality of peptides or polypeptides comprising epitope or antigen derived from a COVID-19 coronavirus or variant thereof,
wherein the product of manufacture has affixed or attached thereon at least one viral nuclear antigen from the COVID-19 coronavirus or variant thereof
and
(b) (i) at least one peptide or polypeptide epitope or antigen derived from a non-COVID-19 virus, and/or
(ii) a plurality of peptide or polypeptide epitopes or antigens derived from at least one of: a respiratory virus.

2. A multiplexed system comprising:
a product of manufacture of claim 1, and
a digital fluorescent reader, and
(c) a transmitting element capable of transmitting data generated by the digital fluorescent reader and captured or read by the digital camera to a computer, data storage device or a cell phone or equivalents.

3. A method for determining if an individual is positive or seropositive for an antibody against a coronavirus comprising:
(a) applying to or contacting a product of manufacture as set forth in claim 1, one fluid or a plurality of fluids samples from an individual; and
(b) detecting whether, or not, an antibody from the individual specifically binds to one or more of the peptide or polypeptide antigens or epitopes affixed or attached to the surface of the product of manufacture, and
wherein if an antibody from the individual specifically binds to one or more of the peptide or polypeptide antigens or epitopes, then it is determined that the individual is positive or seropositive for an antibody against a coronavirus.

4. The method of claim 3, wherein at least one peptide or polypeptide epitope or antigen derived from a coronavirus comprises or is a coronavirus S1 spike protein domain or a coronavirus receptor-binding domain (RBD), and if the individual is positive or seropositive for antibodies that specifically bind to the coronavirus S1 and/or the coronavirus RBD domain of the coronavirus spike protein, the method comprises determining that the individual has immunity to the coronavirus.

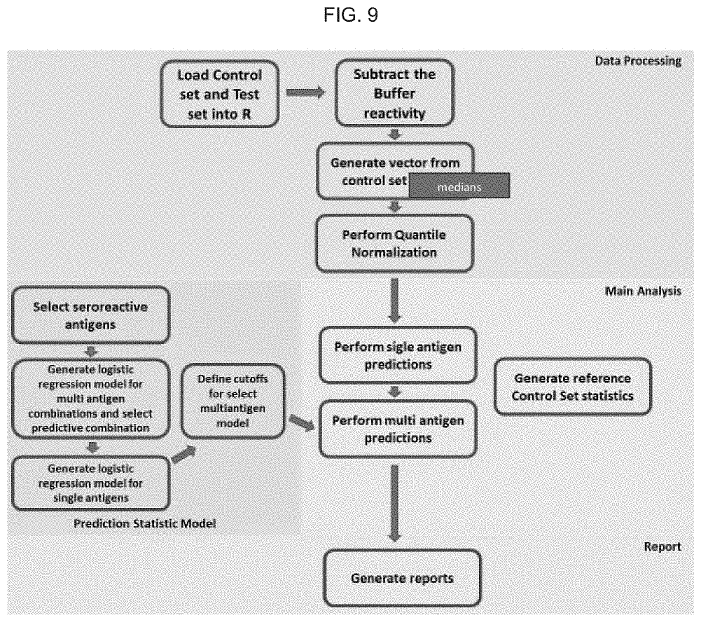

5. The method of claim 3, where the results of detecting whether, or not, an antibody from the individual specifically binds to one or more of the peptide or polypeptide antigens or epitopes affixed or attached to the surface of the product of manufacture comprises use of a data analysis computer-executed program as described in FIG. 8 or FIG. 9.

6. A pharmaceutical formulation or a vaccine comprising a peptide or polypeptide derived from the coronavirus S1 and/or the coronavirus RBD domain of the coronavirus spike protein.

7. A method for generating antibodies protective to a coronavirus comprising administering to an individual in need thereof a vaccine or claim 6.

8-11. (canceled)

12. The product of manufacture of claim 1, wherein the coronavirus is the strain COVID-19 or variant thereof, and the product of manufacture has affixed or attached thereon at least one viral nuclear antigen from the COVID-19 coronavirus strain or variant thereof.

13. The product of manufacture of claim 12, wherein the at least one viral nuclear antigen comprises a coronavirus N nucleoprotein or coronavirus nonstructural protein 1 (nsp1).

14. The product of manufacture of claim 12, The product of manufacture of claim 12, wherein the COVID-19 coronavirus strain is an L-type COVID-19 coronavirus strain and/or an S-type COVID-19 coronavirus strain.

15. The product of manufacture of claim 12, wherein the non-COVID-19 virus is a virus of the subfamily Orthocoronavirinae, or the family Coronaviridae, or the order Nidovirales.

16. The product of manufacture of claim 12, wherein the non-COVID-19 coronavirus is the SARS coronavirus (SARS-CoV) that causes severe acute respiratory syndrome (SARS), and/or the MERS-coronavirus (MERS-CoV) that causes Middle East respiratory syndrome (MERS).

17. The product of manufacture of claim 1, wherein the at least one peptide or polypeptide epitope or antigen derived from a coronavirus, optionally a coronavirus COVID-19 virus, comprises or is: a coronavirus S1, S2 or S1 and S2 spike protein domain; a coronavirus receptor-binding domain (RBD); and/or, a coronavirus nucleocapsid protein (NP) protein.

18. The product of manufacture of claim 1, wherein the respiratory virus is an influenza virus, rhinovirus, a picornavirus, an adenovirus, a human respiratory syncytial virus, an orthopneumovirus, an enterovirus other than a rhinovirus, a human parainfluenza virus and/or a human metapneumovirus.

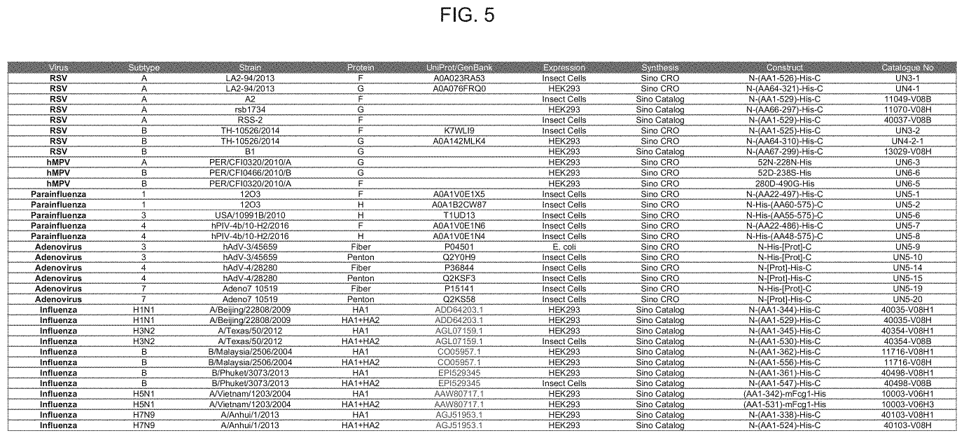

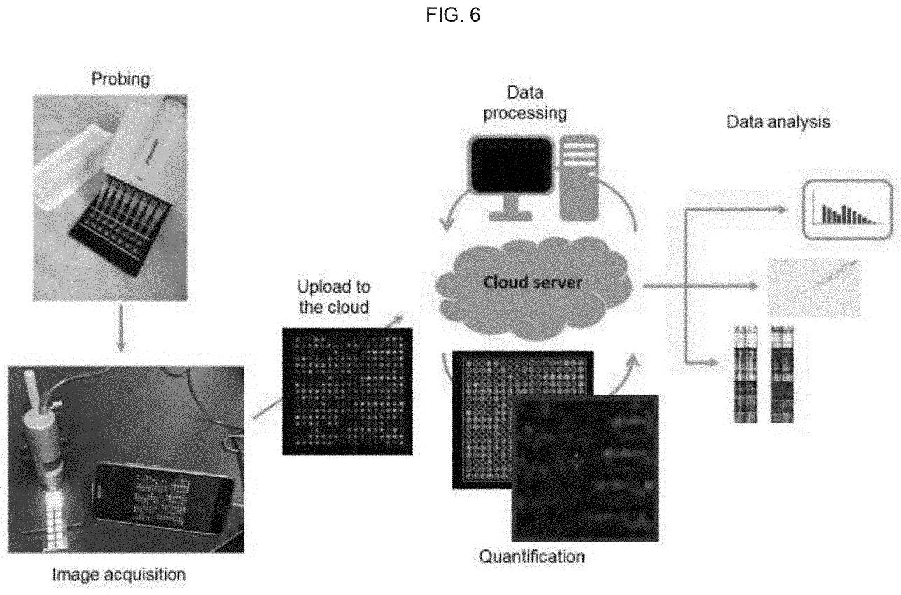

19. The product of manufacture of claim 1, wherein the product of manufacture comprises, or has affixed or attached onto one of its surfaces, at least one, several substantially most of or all of the peptide or polypeptide antigens or epitopes, or antigenic fragments thereof; as set forth in Table 1 (FIG. 3) , Table 2 (FIG. 4) and/or Table 3 (FIG. 5); or the table of FIG. 25, and/or, one, several or all of SEQ ID NO:1 to SEQ ID NO:61, or any antigenic fragment thereof.

20. The multiplexed system of claim 2, wherein the digital fluorescent reader comprises a digital fluorescent microscope comprising a digital camera or equivalent.

21. The multiplexed system of claim 2, wherein the multiplexed system comprises a data analysis computer-executed program as described in FIG. 8 or FIG. 9.

22. The method of claim 3, wherein the one fluid or the plurality of fluids comprises a blood, tears, cerebral spinal fluid (CSF) or a serum sample from an individual.

23. The pharmaceutical formulation or a vaccine of claim 6, wherein the coronavirus is a strain COVID-19,
wherein optionally the peptide or polypeptide is derived from the coronavirus S1 and/or the coronavirus RBD domain of the coronavirus spike protein comprises or consists of an amino acid sequence as set forth in SEQ ID NO: 1, SEQ ID NO:2 or SEQ ID NO:3, or an antigenic fragment thereof.

24. The pharmaceutical formulation or a vaccine of claim 6, wherein the pharmaceutical formulation or vaccine is formulated as a peptide- or polypeptide-comprising pharmaceutical formulation or vaccine, or as a peptide-encoding or polypeptide-encoding nucleic acid-comprising pharmaceutical formulation or vaccine,
and optionally the nucleic acid is a DNA or an RNA molecule, and optionally the nucleic acid is contained in a delivery vehicle,
and optionally the delivery vehicle is a viral vector,
and optionally the nucleic acid comprises an RNA contained in a liposome, a lipid nanoparticle (LNP) or a nanoliposome.


SEQUENCE LISTING (61 sequences)

SEQ ID NO: 1 - protein, 433 aa, SARS coronavirus Tor2
  misc_feature (1)..(433): Antigen: SARS-CoV_NP, Virus: SARS-CoV, Strain:SARS, Uniprot/Genbank: NP_828858.1, Expression: Insect, Cells, SinoCatalog: 40143-V08B
MSDNGPQSNQRSAPRITFGGPTDSTDNNQNGGRNGARPKQRRPQGLPNNTASWFTALTQHGKEELRFPRGQGVPINTNSGPDDQIGYYRRATRRVRGGDGKMKELSPRWYFYYLGTGPEASLPYGANKEGIVWVATEGALNTPKDHIGTRNPNNNAATVLQLPQGTTLPKGFYAEGSRGGSQASSRSSSRSRGNSRNSTPGSSRGNSPARMASGGGETALALLLLDRLNQLESKVSGKGQQQQGQTVTKKSAAEASKKPRQKRTATKQYNVTQAFGRRGPEQTQGNFGDQDLIRQGTDYKHWPQIAQFAPSASAFFGMSRIGMEVTPSGTWLTYHGAIKLDDKDPQFKDNVILLNKHIDAYKTFPPTEPKKDKKKKTDEAQPLPQRQKKQPTVTLLPAADMDDFSRQLQNSMSGASADSTQAAHHHHHHHHHH

SEQ ID NO: 2 - protein, 1273 aa, Artificial Sequence
  Synthetic
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 3 - protein, 670 aa, Artificial Sequence
  Synthetic
VNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRAR

SEQ ID NO: 4 - protein, 223 aa, Artificial Sequence
  Synthetic
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 5 - protein, 459 aa, Dromedary camel coronavirus HKU23
  misc_feature (1)..(459): Antigen: DcCoV_HKU23-368F_NP, Virus: CommonCoV, Strain:HKU23-368F, Uniprot/Genbank: AHN64796.1, Expression: HEK293, SinoCatalog: 40458-V08B
MSFTPGKQSSSRASSGNRSGNGILKWADQSDQSRNVQTRGRRAQPKQTVTSQQPSGGNVVPYYSWFSGITQFQKGKEFEFAEGQGVPIAPGVPATEAKGYWYRHNRRSFKTADGNQRQLLPRWYFYYLGTGPHAKDQYGTDIDGVFWVASNQADVNTPADILDRDPSSDEAIPTRFPPGTVLPQGYYIEGSGRSAPNSRSTSRASSRASSAGSRSRANSGNRTPTSGVTPDMADQIASLVLAKLGKDATKPQQVTKQTAKEIRQKILNKPRQKRSPNKQCTVQQCFGKRGPNQNFGGGEMLKLGTSDPQFPILAELAPTAGAFFFGSRLELAKVQNLSGNLDEPQKDVYELRYNGAIRFDSTLSGFETIMKVLNDNLNAYQQQDGMMNMSPKPQRQRGQKNGQGENDNISVAAPKSRVQQNKSRELTAEDISLLKKMDEPFTENTSEIAHHHHHHHHHH

SEQ ID NO: 6 - protein, 362 aa, Influenza B virus
  misc_feature (1)..(362): Antigen: B_B/Malaysia/2506/2004_HA1, Virus: Influenza, Strain:B/Malaysia/2506/2004, Uniprot/Genbank: CO05957.1, Expression: HEK293, SinoCatalog: 11716-V08H1
MKAIIVLLMVVTSNADRICTGITSSNSPHVVKTATQGEVNVTGVIPLTTTPTKSHFANLKGTETRGKLCPKCLNCTDLDVALGRPKCTGNIPSARVSILHEVRPVTSGCFPIMHDRTKIRQLPNLLRGYEHIRLSTHNVINAENAPGGPYKIGTSGSCPNVTNGNGFFATMAWAVPKNDNNKTATNSLTIEVPYICTEGEDQITVWGFHSDNEIQMAKLYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVKTPLKLANGTKYRPPAKLLKER

SEQ ID NO: 7 - protein, 556 aa, Influenza B virus
  misc_feature (1)..(556): Antigen: B_B/Malaysia/2506/2004_HA1+HA2, Virus: Influenza, Strain:B/Malaysia/2506/2004, Uniprot/Genbank: CO05957.1, Expression: HEK293, SinoCatalog: 11716-V08H
MKAIIVLLMVVTSNADRICTGITSSNSPHVVKTATQGEVNVTGVIPLTTTPTKSHFANLKGTETRGKLCPKCLNCTDLDVALGRPKCTGNIPSARVSILHEVRPVTSGCFPIMHDRTKIRQLPNLLRGYEHIRLSTHNVINAENAPGGPYKIGTSGSCPNVTNGNGFFATMAWAVPKNDNNKTATNSLTIEVPYICTEGEDQITVWGFHSDNEIQMAKLYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTA

SEQ ID NO: 8 - protein, 566 aa, Influenza A virus
  misc_feature (1)..(566): Antigen: H1N1_A/Beijing/22808/2009_HA1, Virus: Influenza, Strain:A/Beijing/22808/2009, Uniprot/Genbank: ADD64203.1, Expression: HEK293, SinoCatalog: 40035-V08H1
MKAILVVLLYTFATANADTLCIGYHANNSTDTVDTVLEKNVTVTHSVNLLEDKHNGKLCKLRGVAPLHLGKCNIAGWILGNPECESLSTASSWSYIVETSSSDNGTCYPGDFIDYEELREQLSSVSSFERFEIFPKTSSWPNHDSNKGVTAACPHAGAKSFYKNLIWLVKKGNSYPKLSKSYINDKGKEVLVLWGIHHPSTSADQQSLYQNADAYVFVGTSRYSKKFKPEIAIRPKVRDQEGRMNYYWTLVEPGDKITFEATGNLVVPRYAFAMERNAGSGIIISDTPVHDCNTTCQTPKGAINTSLPFQNIHPITIGKCPKYVKSTKLRLATGLRNVPSIQSRGLFGAIAGFIEGGWTGMVDGWYGYHHQNEQGSGYAADLKSTQNAIDKITNKVNSVIEKMNTQFTAVGKEFNHLEKRIENLNKKVDDGFLDIWTYNAELLVLLENERTLDYHDSNVKNLYEKVRSQLKNNAKEIGNGCFEFYHKCDNTCMESVKNGTYDYPKYSEEAKLNREEIDGVKLESTRIYQILAIYSTVASSLVLVVSLGAISFWMCSNGSLQCRICI

SEQ ID NO: 9 - protein, 566 aa, Influenza A virus; identical to SEQ ID NO: 8
  misc_feature (1)..(566): Antigen: H1N1_A/Beijing/22808/2009_HA1+HA2, Virus: Influenza, Strain:A/Beijing/22808/2009, Uniprot/Genbank: ADD64203.1, Expression: HEK293, SinoCatalog: 40035-V08H

SEQ ID NO: 10 - protein, 566 aa, Influenza A virus
  misc_feature (1)..(566): Antigen: H3N2_A/Texas/50/2012_HA1, Virus: Influenza, Strain:A/Texas/50/2012, Uniprot/Genbank: AGL07159.1, Expression: HEK293, SinoCatalog: 40354-V08H1
MKTIIALSYILCLVFAQKLPGNDNSTATLCLGHHAVPNGTIVKTITNDRIEVTNATELVQNSSIGEICDSPHQILDGENCTLIDALLGDPQCDGFQNKKWDLFVERSKAYSNCYPYDVPDYASLRSLVASSGTLEFNNESFNWNGVTQNGTSSACIRRSNNSFFSRLNWLTHLNFKYPALNVTMPNNEQFDKLYIWGVHHPGTDKDQIFLYAQPSGRITVSTKRSQQAVIPNIGSRPRIRNIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPEKQTRGIFGAIAGFIENGWEGMVDGWYGFRHQNSEGRGQAADLKSTQAAIDQINGKLNRLIGKTNEKFHQIEKEFSEVEGRIQDLEKYVEDTKIDLWSYNAELLVALENQHTIDLTDSEMNKLFEKTKKQLRENAEDMGNGCFKIYHKCDNACIGSIRNGTYDHDVYRDEALNNRFQIKGVELKSGYKDWILWISFAISCFLLCVALLGFIMWACQKGNIRCNICI

SEQ ID NO: 11 - protein, 566 aa, Influenza A virus; identical to SEQ ID NO: 10
  misc_feature (1)..(566): Antigen: H3N2_A/Texas/50/2012_HA1+HA2, Virus: Influenza, Strain:A/Texas/50/2012, Uniprot/Genbank: AGL07159.1, Expression: Insect, Cells, SinoCatalog: 40354-V08B

SEQ ID NO: 12 - protein, 568 aa, Influenza A virus
  misc_feature (1)..(568): Antigen: H5N1_A/Vietnam/1203/2004_HA1, Virus: Influenza, Strain:A/Vietnam/1203/2004, Uniprot/Genbank: AAW80717.1, Expression: HEK293, SinoCatalog: 10003-V06H1
MEKIVLLFAIVSLVKSDQICIGYHANNSTEQVDTIMEKNVTVTHAQDILEKKHNGKLCDLDGVKPLILRDCSVAGWLLGNPMCDEFINVPEWSYIVEKANPVNDLCYPGDFNDYEELKHLLSRINHFEKIQIIPKSSWSSHEASLGVSSACPYQGKSSFFRNVVWLIKKNSTYPTIKRSYNNTNQEDLLVLWGIHHPNDAAEQTKLYQNPTTYISVGTSTLNQRLVPRIATRSKVNGQSGRMEFFWTILKPNDAINFESNGNFIAPEYAYKIVKKGDSTIMKSELEYGNCNTKCQTPMGAINSSMPFHNIHPLTIGECPKYVKSNRLVLATGLRNSPQRERRRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGIYQILSIYSTVASSLALAIMVAGLSLWMCSNGSLQCRICI

SEQ ID NO: 13 - protein, 568 aa, Influenza A virus; identical to SEQ ID NO: 12
  misc_feature (1)..(568): Antigen: H5N1_A/Vietnam/1203/2004_HA1+HA2, Virus: Influenza, Strain:A/Vietnam/1203/2004, Uniprot/Genbank: AAW80717.1, Expression: HEK293, SinoCatalog: 10003-V06H3

SEQ ID NO: 14 - protein, 560 aa, Influenza A virus
  misc_feature (1)..(560): Antigen: H7N9_A/Anhui/1/2013_HA1, Virus: Influenza, Strain:A/Anhui/1/2013, Uniprot/Genbank: AGJ51953.1, Expression: HEK293, SinoCatalog: 40103-V08H1
MNTQILVFALIAIIPTNADKICLGHHAVSNGTKVNTLTERGVEVVNATETVERTNIPRICSKGKRTVDLGQCGLLGTITGPPQCDQFLEFSADLIIERREGSDVCYPGKFVNEEALRQILRESGGIDKEAMGFTYSGIRTNGATSACRRSGSSFYAEMKWLLSNTDNAAFPQMTKSYKNTRKSPALIVWGIHHSVSTAEQTKLYGSGNKLVTVGSSNYQQSFVPSPGARPQVNGLSGRIDFHWLMLNPNDTVTFSFNGAFIAPDRASFLRGKSMGIQSGVQVDANCEGDCYHSGGTIISNLPFQNIDSRAVGKCPRYVKQRSLLLATGMKNVPEIPKGRGLFGAIAGFIENGWEGLIDGWYGFRHQNAQGEGTAADYKSTQSAIDQITGKLNRLIEKTNQQFELIDNEFNEVEKQIGNVINWTRDSITEVWSYNAELLVAMENQHTIDLADSEMDKLYERVKRQLRENAEEDGTGCFEIFHKCDDDCMASIRNNTYDHSKYREEAMQNRIQIDPVKLSSGYKDVILWFSFGASCFILLAIVMGLVFICVKNGNMRCTICI

SEQ ID NO: 15 - protein, 560 aa, Influenza A virus; identical to SEQ ID NO: 14
  misc_feature (1)..(560): Antigen: H7N9_A/Anhui/1/2013_HA1+HA2, Virus: Influenza, Strain:A/Anhui/1/2013, Uniprot/Genbank: AGJ51953.1, Expression: HEK293, SinoCatalog: 40103-V08H

SEQ ID NO: 16 - protein, 319 aa, Human adenovirus B3
  misc_feature (1)..(319): Antigen: hAdV-3/45659_Fiber, Virus: Adenovirus, Strain:hAdV-3/45659, Uniprot/Genbank: P04501, Expression: E., coli, SinoCatalog: UN5-9
MAKRARLSTSFNPVYPYEDESSSQHPFINPGFISPDGFTQSPNGVLSLKCVNPLTTASGSLQLKVGSGLTVDTTDGSLEENIKVNTPLTKSNHSINLPIGNGLQIEQNKLCSKLGNGLTFDSSNSIALKNNTLWTGPKPEANCIIEYGKQNPDSKLTLILVKNGGIVNGYVTLMGASDYVNTLFKNKNVSINVELYFDATGHILPDSSSLKTDLELKYKQTADFSARGFMPSTTAYPFVLPNAGTHNENYIFGQCYYKASDGALFPLEVTVMLNKRLPDSRTSYVMTFLWSLNAGLAPETTQATLITSPFTFSYIREDD

SEQ ID NO: 17 - protein, 544 aa, Human adenovirus B3
  misc_feature (1)..(544): Antigen: hAdV-3/45659_Penton, Virus: Adenovirus, Strain:hAdV-3/45659, Uniprot/Genbank: Q2Y0H9, Expression: Insect, Cells, SinoCatalog: UN5-10
MRRRAVLGGAVVYPEGPPPSYESVMQQQAAMIQPPLEAPFVPPRYLAPTEGRNSIRYSELSPLYDTTKLYLVDNKSADIASLNYQNDHSNFLTTVVQNNDFTPTEASTQTINFDERSRWGGQLKTIMHTNMPNVNEYMFSNKFKARVMVSRKAPEGVTVNDTYDHKEDILKYEWFEFILPEGNFSATMTIDLMNNAIIDNYLEIGRQNGVLESDIGVKFDTRNFRLGWDPETKLIMPGVYTYEAFHPDIVLLPGCGVDFTESRLSNLLGIRKRHPFQEGFKIMYEDLEGGNIPALLDVTAYEESKKDTTTETTTLAVAEETSEDDDITRGDTYITEKQKREAAAAEVKKELKIQPLEKDSKSRSYNVLEDKINTAYRSWYLSYNYGNPEKGIRSWTLLTTSDVTCGAEQVYWSLPDMMQDPVTFRSTRQVNNYPVVGAELMPVFSKSFYNEQAVYSQQLRQATSLTHVFNRFPENQILIRPPAPTITTVSENVPALTDHGTLPLRSSIRGVQRVTVTDARRRTCPYVYKALGIVAPRVLSSRTF

SEQ ID NO: 18 - protein, 426 aa, Human adenovirus E4
  misc_feature (1)..(426): Antigen: hAdV-4/28280_Fiber, Virus: Adenovirus, Strain:hAdV-4/28280, Uniprot/Genbank: P36844, Expression: Insect, Cells, SinoCatalog: UN5-14
MSKSARGWSDGFDPVYPYDADNDRPCPSSTLPSFSSDGFQEKPLGVLSLGPGRPCHTKNGEITLKLGEGVDLDDSGKLIANTVNKAIAPLSFFQQHHFPLTWIPLYTPKMENYPYKFLPPLSILKSTILNTLVSAFGSGLGLSGSALAVQLASPLTFDDKGNIKITLNRGLHVTTGDAIESNISWAKGIKFEDGAIATNIGKGSRFGTSSTETGVNNAYPIQVKLGSGLSFDSTGAIMAGNKDYDKLTLWTTPDPSPNCQILAENDAKLTLCLTMCDSQILATVSVLVVRSGNLNPITGTVSSAQVFLRFDANGVLLTEHSTSKKYWGYKQGDSIDGTPYTNAVGFMPNSTAYPKTQSSTTKNNIVGQVYMNGDVSKPMLLTITLNGTDDTTSAYSMSFSYTWTNGSYIGATFGANSYTFSYIAQQ

SEQ ID NO: 19 - protein, 535 aa, Human adenovirus E4
  misc_feature (1)..(535): Antigen: hAdV-4/28280_Penton, Virus: Adenovirus, Strain:hAdV-4/28280, Uniprot/Genbank: Q2KSF3, Expression: Insect, Cells, SinoCatalog: UN5-15
MMRRAYPEGPPPSYESVMQQAMAAAAAIQPPLEAPYVPPRYLAPTEGRNSIRYSELTPLYDTTRLYLVDNKSADIASLNYQNDHSNFLTTVVQNNDFTPTEASTQTINFDERSRWGGQLKTIMHTNMPNVNQFMYSNKFKARVMVSRKTPNGVTVGDNYDGSQDELKYEWVEFELPEGNFSVTMTIDLMNNAIIDNYLAVGRQNGVLESDIGVKFDTRNFRLGWDPVTELVMPGVYTNEAFHPDIVLLPGCGVDFTESRLSNLLGIRKRQPFQEGFQIMYEDLDGGNIPALLDVEAYEKSKEESVAAATTAVATASTEVRDDNFASAAAVAAVKADETKSKIVIQPVEKDSKERSYNVLSDKKNTAYRSWYLAYNYGDRDKGVRSWTLLTTSDVTCGVEQVYWSLPDMMQDPVTFRSTHQVSNYPVVGAELLPVYSKSFFNEQAVYSQQLRAFTSLTHVFNRFPENQILVRPPAPTITTVSENVPALTDHGTLPLRSSIRGVQRVTVTDARRRTCPYVYKALGIVAPRVLSSRTF

SEQ ID NO: 20 - protein, 1171 aa, Human coronavirus 229E
  misc_feature (1)..(1171): Antigen: HCoV-229E_S1, Virus: CommonCoV, Strain:229E, Uniprot/Genbank: A0A1L7B942, Expression: HEK293, SinoCatalog: UN1-2
MFVLLVAYALLHIAGCQTTNGTNTSHSVCNGCVGHSENVFAVESGGYIPSNFSFNNWFLLTNTSSVVDGVVRSFQPLLLNCLWSVSGSQFTTGFVYFNGTGRGACKGFYSNASSDVIRYNINFEENLRRGTILFKTSYGAVVFYCTNNTLVSGDAHIPSGTVLGNFYCFVNTTIGNETTSAFVGALPKTVREFVISRTGHFYINGYRYFSLGDVEAVNFNVTNAATIVCTVALASYADVLVNVSQTAIANIIYCNSVINRLRCDQLSFDVPDGFYSTSPIQPVELPMSIVSLPVYHKHTFIVLHVKFEHQRGPGKCYNCRPSVINITLANFNETKGPLCVDTSHFTTQFVDNVKLARWSASINTGNCPFSFGKVNNFVKFGSVCFSLKDIPGGCAMPIMANLVNHKSHNIGSLYVSWSDGDVITGVPKPVEGVSSFMNVTLNKCTKYNIYDVSGVGVIRISNDTFLNGITYTSTSGNLLGFKDVTNGTIYSITPCNPPDQLVVYQQAVVGAMLSENFTSYGFSNVVEMPKFFYASNGTYNCTDAVLTYSSFGVCADGSIIAVQPRNVSYDSVSAIVTANLSIPSNWTTSVQVEYLQITSKPIVVDCSTYVCNGNVRCVELLKQYTSACKTIEDALRNSAMLESADVSEMLTFDKKAFTLANVSSFGDYNLSSVIPSLPRSGSRVAGRSAIEDILFSKLVTSGLGTVDADYKKCTKGLSIADLACAQYYNGIMVLPGVADAERMAMYTGSLIGGIALGGLTSAASIPFSLAIQSRLNYVALQTDVLQENQKILAASFNKAMTNIVDAFTGVNDAITQTSQALQTVATALNKIQDVVNQQGNSLNHLTSQLRQNFQAISSSIQAIYDRLDIIQADQQVDRLITGRLAALNVFVSHTLTKYTEVRASRQLAQQKVNECVKSQSKRYGFCGNGTHIFSLVNAAPEGLVFLHTVLLPTQYKDVEAWSGLCVDGINGYVLRQPNLALYKEGNYYRITSRIMFEPRIPTIADFVQIENCNVTFVNISRSELQTIVPEYIDVNKTLQELSYKLPNYTVPDLVVEQYNQTILNLTSEISTLENKSAELNYTVQKLQTLIDNINSTLVDLKWLNRVETYIKWPWWVWLCISVVLIFVVSMLLLCCCSTGCCGFFSCFASSIKGCCESTKLPYYDVEKIHIQ

SEQ ID NO: 21 - protein, 1171 aa, Human coronavirus 229E; identical to SEQ ID NO: 20
  misc_feature (1)..(1171): Antigen: HCoV-229E_S1+S2, Virus: CommonCoV, Strain:229E, Uniprot/Genbank: A0A1L7B942, Expression: Insect, Cells, SinoCatalog: UN2-2

SEQ ID NO: 22 - protein, 1351 aa, Human coronavirus HKU1
  misc_feature (1)..(1351): Antigen: HCoV-HKU1_HE, Virus: CommonCoV, Strain:HKU1, Uniprot/Genbank:Q0ZME7, Expression: HEK293, SinoCatalog: UN1-4-1
MFLIIFILPTTLAVIGDFNCTNSFINDYNKTIPRISEDVVDVSLGLGTYYVLNRVYLNTTLLFTGYFPKSGANFRDLALKGSIYLSTLWYKPPFLSDFNNGIFSKVKNTKLYVNNTLYSEFSTIVIGSVFVNTSYTIVVQPHNGILEITACQYTMCEYPHTVCKSKGSIRNESWHIDSSEPLCLFKKNFTYNVSADWLYFHFYQERGVFYAYYADVGMPTTFLFSLYLGTILSHYYVMPLTCNAISSNTDNETLEYWVTPLSRRQYLLNFDEHGVITNAVDCSSSFLSEIQCKTQSFAPNTGVYDLSGFTVKPVATVYRRIPNLPDCDIDNWLNNVSVPSPLNWERRIFSNCNFNLSTLLRLVHVDSFSCNNLDKSKIFGSCFNSITVDKFAIPNRRRDDLQLGSSGFLQSSNYKIDISSSSCQLYYSLPLVNVTINNFNPSSWNRRYGFGSFNLSSYDVVYSDHCFSVNSDFCPCADPSVVNSCAKSKPPSAICPAGTKYRHCDLDTTLYVKNWCRCSCLPDPISTYSPNTCPQKKVVVGIGEHCPGLGINEEKCGTQLNHSSCFCSPDAFLGWSFDSCISNNRCNIFSNFIFNGINSGTTCSNDLLYSNTEISTGVCVNYDLYGITGQGIFKEVSAAYYNNWQNLLYDSNGNIIGFKDFLTNKTYTILPCYSGRVSAAFYQNSSSPALLYRNLKCSYVLNNISFISQPFYFDSYLGCVLNAVNLTSYSVSSCDLRMGSGFCIDYALPSSRRKRRGISSPYRFVTFEPFNVSFVNDSVETVGGLFEIQIPTNFTIAGHEEFIQTSSPKVTIDCSAFVCSNYAACHDLLSEYGTFCDNINSILNEVNDLLDITQLQVANALMQGVTLSSNLNTNLHSDVDNIDFKSLLGCLGSQCGSSSRSLLEDLLFNKVKLSDVGFVEAYNNCTGGSEIRDLLCVQSFNGIKVLPPILSETQISGYTTAATVAAMFPPWSAAAGVPFSLNVQYRINGLGVTMDVLNKNQKLIANAFNKALLSIQNGFTATNSALAKIQSVVNANAQALNSLLQQLFNKFGAISSSLQEILSRLDNLEAQVQIDRLINGRLTALNAYVSQQLSDITLIKAGASRAIEKVNECVKSQSPRINFCGNGNHILSLVQNAPYGLLFIHFSYKPTSFKTVLVSPGLCLSGDRGIAPKQGYFIKQNDSWMFTGSSYYYPEPISDKNVVFMNSCSVNFTKAPFIYLNNSIPNLSDFEAELSLWFKNHTSIAPNLTFNSHINATFLDLYYEMNVIQESIKSLNSSFINLKEIGTYEMYVKWPWYIWLLIVILFIIFLMILFFICCCTGCGSACFSKCHNCCDEYGGHNDFVIKASHDD

SEQ ID NO: 23 - protein, 756 aa, Human coronavirus HKU1
  misc_feature (1)..(756): Antigen: HCoV-HKU1_S1,N-(AA1-760), Virus: CommonCoV, Strain:HKU1, Uniprot/Genbank: YP_173238.1, Expression: HEK293, SinoCatalog: 40021-V08H
GDFNCTNFAINDLNTTVPRISEYVVDVSYGLGTYYILDRVYLNTTILFTGYFPKSGANFRDLSLKGTTYLSTLWYQKPFLSDFNNGIFSRVKNTKLYVNKTLYSEFSTIVIGSVFINNSYTIVVQPHNGVLEITACQYTMCEYPHTICKSKGSSRNESWHFDKSEPLCLFKKNFTYNVSTDWLYFHFYQERGTFYAYYADSGMPTTFLFSLYLGTLLSHYYVLPLTCNAISSNTDNETLQYWVTPLSKRQYLLKFDNRGVITNAVDCSSSFFSEIQCKTKSLLPNTGVYDLSGFTVKPVATVHRRIPDLPDCDIDKWLNNFNVPSPLNWERKIFSNCNFNLSTLLRLVHTDSFSCNNFDESKIYGSCFKSIVLDKFAIPNSRRSDLQLGSSGFLQSSNYKIDTTSSSCQLYYSLPAINVTINNYNPSSWNRRYGFNNFNLSSHSVVYSRYCFSVNNTFCPCAKPSFASSCKSHKPPSASCPIGTNYRSCESTTVLDHTDWCRCSCLPDPITAYDPRSCSQKKSLVGVGEHCAGFGVDEEKCGVLDGSYNVSCLCSTDAFLGWSYDTCVSNNRCNIFSNFILNGINSGTTCSNDLLQPNTEVFTDVCVDYDLYGITGQGIFKEVSAVYYNSWQNLLYDSNGNIIGFKDFVTNKTYNIFPCYAGRVSAAFHQNASSLALLYRNLKCSYVLNNISLTTQPYFDSYLGCVFNADNLTDYSVSSCALRMGSGFCVDYNSPSSSSSRRKRRAHHHHHHHHHH

SEQ ID NO: 24 - protein, 1351 aa, Human coronavirus HKU1; identical to SEQ ID NO: 22
  misc_feature (1)..(1351): Antigen: HCoV-HKU1_S1,N-(AA13-756), Virus: CommonCoV, Strain:HKU1, Uniprot/Genbank: Q0ZME7, Expression: HEK293, SinoCatalog: UN1-3

SEQ ID NO: 25 - protein, 1351 aa, Human coronavirus HKU1; identical to SEQ ID NO: 22
  misc_feature (1)..(1351): Antigen: HCoV-HKU1_S1+S2, Virus: CommonCoV, Strain:HKU1, Uniprot/Genbank: Q0ZME7, Expression: Insect, Cells, SinoCatalog: UN2-3

SEQ ID NO: 26 - protein, 1356 aa, Human coronavirus NL63
  misc_feature (1)..(1356): Antigen: HCoV-NL63_S1, Virus: CommonCoV, Strain:NL63, Uniprot/Genbank: A0A1L2YVI8, Expression: HEK293, SinoCatalog: UN1-1
MKLFLILLVLPLASCFFTCNSNANLSMLQLGVPDNSSTIVTGLLPTHWICANQSTSVYSANGFFYIDVGNHRSAFALHTGYYDVNQYYIYVTNEIGLNASVTLKICKFGINTTFDFLSNSSSSFDCIVNLLFTEQLGAPLGITISGETVRLHLYNVTRTFYVPAAYKLTKLSVKCYFNYSCVFSVVNATVTVNVTTHNGRVVNYTVCDDCNGYTDNIFSVQQDGRIPNGFPFNNWFLLTNGSTLVDGVSRLYQPLRLTCLWPVPGLKSSTGFVYFNATGSDVNCNGYQHNSVADVMRYNLNFSANSVDNLKSGVIVFKTLQYDVLFYCSNSSSGVLDTTIPFGPSSQPYYCFINSTINTTHVSTFVGVLPPTVREIVVARTGQFYINGFKYFDLGFIEAVNFNVTTASATDFWTVAFATFVDVLVNVSATKIQNLLYCDSPFEKLQCEHLQFGLQDGFYSANFLDDNVLPETYVALPIYYQHTDINFTATASFGGSCYVCKPHQVNISLNGNTSVCVRTSHFSIRYIYNRVKSGSPGDSSWHIYLKSGTCPFSFSKLNNFQKFKTICFSTVAVPGSCNFPLEATWHYTSYTIVGALYVTWSEGNSITGVPYPVSGIREFSNLVLNNCTKYNIYDYVGTGIIRSSNQSLAGGITYVSNSGNLLGFKNVSTGNIFIVTPCNQPDQVAVYQQSIIGAMTAVNESRYGLQNLLQLPNFYYVSNGGNNCTTAVMTYSNFGICADGSLIPVRLRNSSDNGISAIITANLSIPSNWTTSVQVEYLQITSIPIVVDCATYVCNGNPRCKNLLKQYTSACKTIEDALRLSAHLETNDVSSMLTFDSNAFSLANVTSFGDYNLSSVLPQRNIHSSRIAGRSALEDLLFSKVVTSGLGTVDVDYKSCTKGLSIADLACAQYYNGIMVLPGVADAERMAMYTGSLIGGMVLGGLTSAAAIPFSLALQARLNYVALQTDVLQENQKILAASFNKAINNIVASFSSVNDAITQTAEAIHTVTIALNKIQDVVNQQGSALNHLTSQLRHNFQAISNSIQAIYDRLDSIQADQQVDRLITGRLAALNAFVSQVLNKYTEVRSSRRLAQQKINECVKSQSNRYGFCGNGTHIFSIVNSAPDGLLFLHTVLLPTDYKNVKAWSGICVDGIYGYVLRQPNLVLYSDNGVFRVTSRVMFQPRLPVLSDFVQIYNCNVTFVNISRVELHTVIPDYVDVNKTLQEFAQNLPKYVKPNFDLTPFNLTYLNLSSELKQLEAKTASLFQTTVELQGLIDQINSTYVDLKLLNRFENYIKWPWWVWLIISVVFVVLLSLLVFCCLSTGCCGCCNCLTSSMRGCCDCGSTKLPYYEFEKVHVQ

SEQ ID NO: 27 - protein, 1356 aa, Human coronavirus NL63; identical to SEQ ID NO: 26
  misc_feature (1)..(1356): Antigen: HCoV-NL63_S1+S2, Virus: CommonCoV, Strain:NL63, Uniprot/Genbank: A0A1L2YVI8, Expression: Insect, Cells, SinoCatalog: UN2-1

SEQ ID NO: 28 - protein, 427 aa, Human coronavirus OC43
  misc_feature (1)..(427): Antigen: HCoV-OC43_HE, Virus: CommonCoV, Strain:OC43, Uniprot/Genbank: ATN39879.2, Expression: HEK293, SinoCatalog: UN1-6
MFLLPRFILVSCVIGSLGFYNPPTNVVSHVNGDWFLFGDSRSDCNHIVNINPHNYSYMDLNPALCDSGKISSKAGNSIFRSFHFTDFYNYTGEGQQIIFYEGVNFTPYHAFKCNRSGSNDIWMQNKGLFYTQVYKNMAVYRSLTFVNVSYVYNGSAQATSFCKSGSKLKNLVLNNPAYIAPQAKSGIITKVEADFYLSGCDEYIVPLCIFNGKSLSNTKYYDHSQYYFNKDTGVIYGLNSTETITTSFDLNCHYLVLPSGNYLAISNELLLTVPTKAICFNKRKDFTPVQVVDSRWNNARQSDNMTAVACQPPYCYFRNSTTNYVGVYDINHGDAGFTSILSGLLYNSSCFSQQGVFRYDNISSVWPLYPYGRCPTAAYINIPDLPICVYDPLPVILLGILLGVAIVIIVVLLLHFMVDNVTRLHDA

SEQ ID NO: 29 - protein, 1360 aa, Human coronavirus OC43
  misc_feature (1)..(1360): Antigen: HCoV-OC43_S1, Virus: CommonCoV, Strain:OC43, Uniprot/Genbank: AVR40344.1, Expression: HEK293, SinoCatalog: UN1-5
MFLILLISLPTAFAVIGDLNCTLDPRLKGSFNNRDTGPPSISIDTVDVTNGLGTYYVLDRVYLNTTLFLNGYYPTSGSTYRNMALKGTDLLSTLWFKPPFLSDFINGIFAKVKNTKVFKDGVMYSEFPAITIGSTFVNTSYSVVVQPRTINSTQDGVNKLQGLLEVSVCQYNMCEYPHTICHPNLGNHFKELWHYDTGVVSCLYKRNFTYDVNATYLYFHFYQEGGTFYAYFTDTGFVTKFLFNVYLGMALSHYYVMPLTCIRRPKDGFSLEYWVTPLTPRQYLLAFNQDGIIFNAVDCMSDFMSEIKCKTQSIAPPTGVYELNGYTVQPVADVYRRKPDLPNCNIEAWLNDKSVPSPLNWERKTFSNCNFNMSSLMSFIQADSFTCNNIDAAKIYGMCFSSITIDKFAIPNRRKVDLQLGNLGYLQSSNYRIDTTATSCQLYYNLPAANVSVSRFNPSTWNKRFGFIEDSVFVPQPTGVFTNHSVVYAQHCFKAPKNFCPCSSCSCPGKNNGIGTCPAGTNSLTCDNLCTLDPITLKAPDTYKCPQSKSLVGIGEHCSGLAVKSDYCGNNSCTCQPQAFLGWSADSCLQGDKCNIFANFILHDVNNGLTCSTDLQKANTEIELGVCVNYDLYGISGQGIFVEVNATYYNSWQNLLYDSNGNLYGFRDYITNRTFMIHSCYSGRVSAAYHANSSEPALLFRNIKCNYVFNNSLTRQLQPINYSFDSYLGCVVNAYNSTAISVQTCDLTVGSGYCVDYSKNRRSRRAITTGYRFTNFEPFTVNSVNDSLEPVGGLYEIQIPSEFTIGNMEEFIQTSSPKVTIDCAAFVCGDYAACKLQLVEYGSFCDNINAILTEVNELLDTTQLQVANSLMNGVTLSTKLKDGVNFNVDDINFSPVLGCLGSECSKASSRSAIEDLLFDKVKLSDVGFVEAYNNCTGGAEIRDLICVQSYKGIKVLPPLLSENQISGYTLAATSASLFPPWTAAAGVPFYLNVQYRINGLGVTMDVLSQNQKLIANAFNNALHAIQQGFDATNSALVKIQAVVNANAEALNNLLQQLSNRFGAISASLQEILSRLDALEAEAQIDRLINGRLTALNAYVSQQLSDSTLVKFSAAQAMEKVNECVKSQSSRINFCGNGNHIISLVQNAPYGLYFIHFNYVPTKYVTAKVSPGLCIAGNRGIAPKSGYFVNVNNTWMYTGSGYYYPEPITENNVVVMSTCAVNYTKAPYVMLNTSIPNLPDFKEELDQWFKNQTSVAPDLSLDYINVTFLDLQVEMNRLQEAIKVLNHSYINLKDIGTYEYYVKWPWYVWLLICLAGVAMLVLLFFICCCTGCGTSCFKKCGGCCDDYTGYQELVIKTSHDD

SEQ ID NO: 30 - protein, 1360 aa, Human coronavirus OC43; identical to SEQ ID NO: 29
  misc_feature (1)..(1360): Antigen: HCoV-OC43_S1+S2, Virus: CommonCoV, Strain:OC43, Uniprot/Genbank: AVR40344.1, Expression: Insect, Cells, SinoCatalog: UN2-4

SEQ ID NO: 31 - protein, 228 aa, Human metapneumovirus
  misc_feature (1)..(228): Antigen: hMPV-A_G(52N-228N), Virus: hMPV, Strain:PER/CFI0320/2010/A, Glycoprotein, Uniprot/Genbank: X4XYT2, Expression: HEK293, SinoCatalog: UN6-3
MEVKVENIRAIDMLKARVKNRVARSKCFKNASLILIGITTLSIALNIYLIINYTIQKTTSESEHHTSSPPTESNKETSTIPTDNPDITPNSQHPTQQSTESPTLHPASSTSPPETEPASTPGTTNRPSFVDRSTTQSSESRTKTNSTVHKRNISSTTPRTQFPPRTTTKAVSRTTAFRTSSTGERPTTTSVQSDSSTTARNHEETGSANPQASASTMQTQHTNIAKPN

SEQ ID NO: 32 - protein, 539 aa, Human metapneumovirus
  misc_feature (1)..(539): Antigen: hMPV-B_F(280D-490G), Virus: hMPV, Strain:PER/CFI0320/2010/A, Glycoprotein, Uniprot/Genbank: F4YZY2, Expression: HEK293, SinoCatalog: UN6-5
MSWKVMIIISLLITPQHGLKESYLEESCSTITEGYLSVLRTGWYTNVFTLEVGDVENLTCTDGPSLIKTELDLTKSALRELKTVSADQLAREEQIENPRQSRFVLGAIALGVATAAAVTAGIAIAKTIRLESEVNAIKGALKTTNEAVSTLGNGVRVLATAVRELKEFVSKNLTSAINKNKCDIADLKMAVSFSQFNRRFLNVVRQFSDNAGITPAISLDLMNDAELARAVSYMPTSAGQIKLMLENRAMVRRKGFGILIGVYGSSVIYMVQLPIFGVINTPCWIIKAAPSCSEKDGNYACLLREDQGWYCKNAGSTVYYPNEKDCETRGDHVFCDTAAGINVAEQSRECNINISTTNYPCKVSTGRHPISMVALSPLGALVACYKGVSCSTGSNQVGIIKQLPKGCSYITNQDADTVTIDNTVYQLSKVEGEQHVIKGRPVSSSFDPIRFPEDQFNVALDQVFESIENSQALVDQSNKILNSAEKGNTGFIIVIILIAVLGLTMISVSIIIIIKKTRKPTGAPPELNGVTNGGFIPHS

SEQ ID NO: 33 - protein, 238 aa, Human metapneumovirus
  misc_feature (1)..(238): Antigen: hMPV-B_G(52D-238S), Virus: hMPV, Strain:PER/CFI0466/2010/B, Glycoprotein, Uniprot/Genbank: X4Y8Q3, Expression: HEK293, SinoCatalog: UN6-6
MEVRVENIRAIDMFKAKMKNRIRSSKCYRNATLILIGLTALSMALNIFLIIDYATLKNMTKVEHCVNMPPVEPSKKTPMTSAVDLNTKLNPQQATQLTTEDSTSLAATSENHLHTGTTPTSDATISQQATDEHTTLLRSINRQTTQTTTEKKPTGAITKKEKETTTRTTSTAATQTLNTTNQISNGREATTTSARSRNGATTQNSDQTIQAADPSSKPYHTQKSTTTAHNADTSSLSS

SEQ ID NO: 34 - protein, 555 aa, Human parainfluenza virus
  misc_feature (1)..(555): Antigen: hPIV-112O3_F, Virus: Parainfluenza, Strain:hPIV-1, 12O3, Uniprot/Genbank: A0A1V0E1X5, Expression: Insect, Cells, SinoCatalog: UN5-1
MQSSEVLLLVYSSLLLSSSLCQIPVDKLSNVGVIINEGKLLKIAGSYESRYIVLSLVPSIDLQDGCGTTQIIQYKNLLNRLLIPLKDALDLQESLITITNDTTVTNDNPQTRFFGAVIGTIALGVATAAQITAGIALAEAREARKDIALIKDSIVKTHNSVEFIQRGIGEQIIALKTLQDFVNDEIRPAIGELRCETTALKLGIKLTQHYSELATAFSSNLGTIGEKSLTVQALSSLYSANITEILSTIKKDKSDIYDIIYTEQVKGTVIDVDLEKYMVTLLVKIPILSEIPGVLIYRASSISYNIEGEEWHVAIPNYIINKASSLGGADVTNCIESKLAYICPRDPTQLIPDNQQKCILGDVSKCPVTKVINNLVPKFAFINGGVVANCIASTCTCGTNRIPVNQDRSKGVTFLTYTNCGLIGINGIELYANKRGRDTTWGNQIIKVGPAVSIRPVDISLNLASATNFLEESKTELMKARAIISAVGGWHNKESTQIIIIIIVCVLIIIICSILYYLYRVRRLLIMINSTNNSPINAYTLESRMKNPYMGNHSN

SEQ ID NO: 35 - protein, 575 aa, Human parainfluenza virus
  misc_feature (1)..(575): Antigen: hPIV-112O3_H, Virus: Parainfluenza, Strain:hPIV-1, 12O3, Uniprot/Genbank: A0A1B2CW87, Expression: Insect, Cells, SinoCatalog: UN5-2
MAEKGKTINSYWSTTRNDNSTVNTHINTPAGRTHIWLLIATAMHATLSLIIMILCIDLIMKQDTCMKTNTMTVSSVNESAKTIKETITELIRQEVISRTINIQSSVQSGIPILLNKQSRDLTQLIEKSCNKRELAQICENTIAIHHADGITPLDPHDFWRCPVGEPLLSNNPNISLLPGPSLLSGSSTISGCVRLPSLSIGDAIYAYSSNLITQGCADIGKSYQVLQLGYISLNSDMYPDLNPVISHTYDINDNRKSCSVIAAGTRGYQLCSLPTVNETTDYSSEGIEDLVFDILDLKGKTKSHRYKNEDITFDHPFSAMYPSVGSGIKIEDTLVFLGYGGLTTPLQGNTKCVISKCPNVNQSVCNDALKITWLKKRQVVNVLIRINNYLSDRPKIVVETIPITQNYLGAEGRLLKLGKKIYIYTRSSGWHSNLQIGSLDINKPMTINWTPHRVLSRPGNPDCNWFNKCPRECISGVYTDAYPLSPDAVNVATTTLYANTSRVNPTIMYSNTSKIINMLRLKNGKLEAAYTTTSCITHFGKGYCFHIVEINQTSLNTFQPMLFKTSIPKVCKITS

SEQ ID NO: 36 - protein, 572 aa, Human parainfluenza virus
  misc_feature (1)..(572): Antigen: hPIV-3USA/10991B/2010_H, Virus: Parainfluenza, Strain:hPIV-3, USA/10991B/2010, Uniprot/Genbank: T1UD13, Expression: Insect, Cells, SinoCatalog: UN5-6
MEYWKHTNHGKDAGNELETSTATHGNKLTNKITYILWTITLVLLSIVFIIVLTNSIKSERARESLLQDINNEFMEVTEKIQVASDNTNDLIQSGVNTRLLTIQSHVQNYIPISLTQQISDLRKFISEITIRNDNQEVPPQRITHDVGIKPLNPDDFWRCTSGLPSLMKTPKIRLMPGPGLLAMPTTVDGCVRTPSLVINDLIYAYTSNLITRGCQDIGKSYQVLQIGIITVNSDLVPDLNPRISHTFNINDNRKSCSLALLNTDVYQLCSTPKVDERSDYASSGIEDIVLDIVNYDGSISTTRFKNNNISFDQPYAALYPSVGPGIYYKGKIIFLGYGGLEHPINENAICNTTGCPGKTQRDCNQASHSPWFSDRRMVNSIIVVDKGLNSVPKLKVWTISMRQNYWGSEGRLLLLGNKIYIYTRSTSWHSKLQLGIIDITDYSDIRIKWTWHNVLSRPGNNECPWGHSCPDGCITGVYTDAYPLNPTGSIVSSVILDSQKSRVNPVITYSTATERVNELAIRNKTLSAGYTTTSCITHYNKGYCFHIVEINHKSLNTFQPMLFKTEIPKSCS

SEQ ID NO: 37 - protein, 579 aa, Human parainfluenza virus
  misc_feature (1)..(579): Antigen: hPIV-4b/10-H2/2016_H, Virus: Parainfluenza, Strain:hPIV-4b/10-H2/2016, Uniprot/Genbank: A0A1V0E1N4, Expression: Insect, Cells, SinoCatalog: UN5-8
MQDSRGNTQIFSQANSMVKRTWRLLFRIVTLILLISIFVLSLIIVLQSTPGNLQSDVDIIRKELDELKENFETTSKSLLSVANQITYDVSVLTPIRQEATETNIIAKIKDHCKDRVVKGESTCTLGHKPLHDVSFLNGFNKFYFTYRDNVQIRLNPLLDYPNFIPTATTPHGCIRIPSFSLSQTHWCYTHNTILRGCEDTASSKQYVSLGTLQTLENGDPYFKVEYSHYLNDRKNRKSCSVVAVLDGCLLYCVIMTKNETENFKDPQLATQLLTYISYNGTIKERIINPPGSSRDWVHISPGVGSGILYSNYIIFPLYGGLMENSMIYNNQSGKYFFPNSTKLPCSNKTSEKITGAKDSYTITYFSGRLIQSAFLICDLRQFLSEDCEILIPSNDHMLVGAEGRLYNIENNIFYYQRGSSWWPYPSLYRIKLNSNKKYPRIIETKFTKIEIAPRPGNKDCPGNKACPKECITGVYQDIWPLSYPNTAFPHKKRAYYTGFYLNNSLARRNPTFYTADNLDYHQQERLGKFNLTAGYSTTTCFKQTTTARLYCLYILEVGDSVIGDFQIFSFLRSIDQAIT

SEQ ID NO: 38 - protein, 424 aa, Human betacoronavirus 2c EMC/2012
  misc_feature (1)..(424): Antigen: MERS-CoV_NP, Virus: MERS, Strain:MERS, Uniprot/Genbank: AFS88943.1, Expression: Insect, Cells, SinoCatalog: 40068-V08B
MASPAAPRAVSFADNNDITNTNLSRGRGRNPKPRAAPNNTVSWYTGLTQHGKVPLTFPPGQGVPLNANSTPAQNAGYWRRQDRKINTGNGIKQLAPRWYFYYTGTGPEAALPFRAVKDGIVWVHEDGATDAPSTFGTRNPNNDSAIVTQFAPGTKLPKNFHIEGTGGNSQSSSRASSLSRNSSRSSSQGSRSGNSTRGTSPGPSGIGAVGGDLLYLDLLNRLQALESGKVKQSQPKVITKKDAAAAKNKMRHKRTSTKSFNMVQAFGLRGPGDLQGNFGDLQLNKLGTEDPRWPQIAELAPTASAFMGMSQFKLTHQNNDDHGNPVYFLRYSGAIKLDPKNPNYNKWLELLEQNIDAYKTFPKKEKKQKAPKEESTDQMSEPPKEQRVQGSITQRTRTRPSVQPGPMIDVNTDAHHHHHHHHHH

SEQ ID NO: 39 - protein, 719 aa, Human betacoronavirus 2c EMC/2012
  misc_feature (1)..(719): Antigen: MERS-CoV_S1,N-(AA1-725,HisTag)_HEK293, Virus: MERS, Strain:MERS, Uniprot/Genbank: AFS88936.1, Expression: HEK293, SinoCatalog: 40069-V08H
YVDVGPDSVKSACIEVDIQQTFFDKTWPRPIDVSKADGIIYPQGRTYSNITITYQGLFPYQGDHGDMYVYSAGHATGTTPQKLFVANYSQDVKQFANGFVVRIGAAANSTGTVIISPSTSATIRKIYPAFMLGSSVGNFSDGKMGRFFNHTLVLLPDGCGTLLRAFYCILEPRSGNHCPAGNSYTSFATYHTPATDCSDGNYNRNASLNSFKEYFNLRNCTFMYTYNITEDEILEWFGITQTAQGVHLFSSRYVDLYGGNMFQFATLPVYDTIKYYSIIPHSIRSIQSDRKAWAAFYVYKLQPLTFLLDFSVDGYIRRAIDCGFNDLSQLHCSYESFDVESGVYSVSSFEAKPSGSVVEQAEGVECDFSPLLSGTPPQVYNFKRLVFTNCNYNLTKLLSLFSVNDFTCSQISPAAIASNCYSSLILDYFSYPLSMKSDLSVSSAGPISQFNYKQSFSNPTCLILATVPHNLTTITKPLKYSYINKCSRLLSDDRTEVPQLVNANQYSPCVSIVPSTVWEDGDYYRKQLSPLEGGGWLVASGSTVAMTEQLQMGFGITVQYGTDTNSVCPKLEFANDTKIASQLGNCVEYSLYGVSGRGVFQNCTAVGVRQQRFVYDAYQNLVGYYSDDGNYYCLRACVSVPVSVIYDKETKTHATLFGSVACEHISSTMSQYSRSTRSMLKRRDSTYGPLQTPVGCVLGLVNSSLFVEAHHHHHHHHHH

SEQ ID NO: 40 - protein, 464 aa, Human betacoronavirus 2c EMC/2012
  misc_feature (1)..(464): Antigen: MERS-CoV_S1-RBD,N-(AA367-606,rFcTag), Virus: MERS, Strain:MERS, Uniprot/Genbank: AFS88936.1, Expression: Insect, Cells, SinoCatalog: 40071-V31B1
EAKPSGSVVEQAEGVECDFSPLLSGTPPQVYNFKRLVFTNCNYNLTKLLSLFSVNDFTCSQISPAAIASNCYSSLILDYFSYPLSMKSDLSVSSAGPISQFNYKQSFSNPTCLILATVPHNLTTITKPLKYSYINKCSRLLSDDRTEVPQLVNANQYSPCVSIVPSTVWEDGDYYRKQLSPLEGGGWLVASGSTVAMTEQLQMGFGITVQYGTDTNSVCPKLEFANDTKIASQLGNCVEYASKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 41 - protein, 354 aa, Human betacoronavirus 2c EMC/2012
  misc_feature (1)..(354): Antigen: MERS-CoV, S1-RBD,, N-(AA383-502,, mFc, Tag), Virus: MERS, Strain:MERS, Uniprot/Genbank: AFS88936.1, Expression: Insect, Cells, SinoCatalog: 40071-V05B
CDFSPLLSGTPPQVYNFKRLVFTNCNYNLTKLLSLFSVNDFTCSQISPAAIASNCYSSLILDYFSYPLSMKSDLSVSSAGPISQFNYKQSFSNPTCLILATVPHNLTTITKPLKYSYINKADDDDKAVPRDSGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 42 - protein, 582 aa, Human betacoronavirus 2c EMC/2012
  misc_feature (1)..(582): Antigen: MERS-CoV_S2, Virus: MERS, Strain:MERS, Uniprot/Genbank: AFS88936.1, Expression: Insect, Cells, SinoCatalog: 40070-V08B
DCKLPLGQSLCALPDTPSTLTPRSVRSVPGEMRLASIAFNHPIQVDQLNSSYFKLSIPTNFSFGVTQEYIQTTIQKVTVDCKQYVCNGFQKCEQLLREYGQFCSKINQALHGANLRQDDSVRNLFASVKSSQSSPIIPGFGGDFNLTLLEPVSISTGSRSARSAIEDLLFDKVTIADPGYMQGYDDCMQQGPASARDLICAQYVAGYKVLPPLMDVNMEAAYTSSLLGSIAGVGWTAGLSSFAAIPFAQSIFYRLNGVGITQQVLSENQKLIANKFNQALGAMQTGFTTTNEAFQKVQDAVNNNAQALSKLASELSNTFGAISASIGDIIQRLDVLEQDAQIDRLINGRLTTLNAFVAQQLVRSESAALSAQLAKDKVNECVKAQSKRSGFCGQGTHIVSFVVNAPNGLYFMHVGYYPSNHIEVVSAYGLCDAANPTNCIAPVNGYFIKTNNTRIVDEWSYTGSSFYAPEPITSLNTKYVAPQVTYQNISTNLPPPLLGNSTGIDFQDELDEFFKNVSTSIPNFGSLTQINTTLLDLTYEMLSLQQVVKALNESYIDLKELGNYTYYNKWPAHHHHHHHHHH

SEQ ID NO: 43 - protein, 574 aa, Respiratory Syncytial Virus
  misc_feature (1)..(574): Antigen: RSV-A_LA2-94/2013_F, Virus: RSV, Strain:LA2-94/2013, Uniprot/Genbank: A0A023RA53, Expression: Insect, Cells, SinoCatalog: UN3-1
MELPILKTNAITTILAAVTLCFTSSQNITEEFYQSTCSAVSKGYLSALRTGWYTSVITIELSNIKENKCNGTDAKVKLIKQELDKYKNAVTELQLLMQSSPAANSRARRELPRFMNYTLNNTKNTNVTLSKKRKRRFLGFLLGVGSAIASGIAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSSNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNIDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLALIAVGLLLYCKARSTPVTLSKDQLSGINNIAFSN

SEQ ID NO: 44 - protein, 321 aa, Respiratory Syncytial Virus
  misc_feature (1)..(321): Antigen: RSV-A_LA2-94/2013_G, Virus: RSV, Strain:LA2-94/2013, Uniprot/Genbank: A0A076FRQ0, Expression: HEK293, SinoCatalog: UN4-1
MSKTKDQRTAKTLERTWDTLNHLLFISSCLYKLNLKSIAQITLSILAMIISTSLIIAAIIFIASANHKVTLTTAIIQDATNQIKNTTPTYLTQNPQLGISFSNLSGTTSQSTTILASTTPSAESTPQSTTVKIKNTTTTQILPSKPTTKQRQNKPQNKPNNDFHFEVFNFVPCSICSNNPTCWAICKRIPNKKPGKKTTTKPTKKPTLNTTKKDPKPQTTKPKEVLTTKPTGKPTINTTRTNIRTTLLTSNTKGNPEHTSQEETLHSTTSEGYPSPSQVYTTSGQEETLHSTTSEGYPSPSQVHTTSEYLSQSLSSSNTTK

SEQ ID NO: 45 - protein, 574 aa, Respiratory Syncytial Virus
  misc_feature (1)..(574): Antigen: RSV-B_TH-10526/2014_F, Virus: RSV, Strain:TH-10526/2014, Uniprot/Genbank: K7WLI9, Expression: Insect, Cells, SinoCatalog: UN3-2
MELLIHRSSAIFLTLAINALYLTSSQNITEEFYQSTCSAVSRGYLSALRTGWYTSVITIELSNIKETKCNGTDTKVKLIKQELDKYKNAVTELQLLMQNTPAVNNRARREAPQYMNYTINTTKNLNVSISKKRKRRFLGFLLGVGSAIASGIAVSKVLHLEGEVNKIKNALQSTNKAVVSLSNGVSVLTSKVLDLKNYINNQLLPIVNQQSCRISNIETVIEFQQKNSRLLEITREFSVNAGVTTPLSTYMLTNSELLSLINDMPITNDQKKLMSSNVQIVRQQSYSIMSIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSDEFDASISQVNEKINQSLAFIRRSDELLHNVNTGKSTTNIMITAIIIVIIVVLLSLIAIGLLLYCKAKNTPVTLSKDQLSGINNIAFSK

SEQ ID NO: 46 - protein, 233 aa, Respiratory Syncytial Virus
  misc_feature (1)..(233): Antigen: RSV-B_B1_G, Virus: RSV, Strain:B1, Uniprot/Genbank: , Expression: HEK293, SinoCatalog: 13029-V08H
HKVTLTTVTVQTIKNHTEKNITTYLTQVPPERVSSSKQPTTTSPIHTNSATTSPNTKSETHHTTAQTKGRTTTSTQTNKPSTKPRLKNPPKKPKDDYHFEVFNFVPCSICGNNQLCKSICKTIPSNKPKKKPTIKPTNKPTTKTTNKRDPKTPAKTTKKETTTNPTKKPTLTTTERDTSTSQSTVLDTTTLEHTIQQQSLHSTTPENTPNSTQTPTASEPSTSNSTQNTQSHA

SEQ ID NO: 47 - protein, 430 aa, SARS coronavirus Tor2
  misc_feature (1)..(430): Antigen: SARS-CoV-2_NP, Virus: SARS-CoV-2, Strain:2019-nCoV, Expression: Insect, Cells, SinoCatalog: 40588-V08B
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTAAIKLDDKDPNFKDQVILLNKHIDAYKTFPPTEPKKDKKKKADETQALPQRQKKQQTVTLLPAADLDDFSKQLQQSMSSADSTQAAHHHHHHHHHH

SEQ ID NO: 48 - skipped

SEQ ID NO: 49 - protein, 681 aa, SARS coronavirus Tor2
  misc_feature (1)..(681): Antigen: SARS-CoV-2_S1, Virus: SARS-CoV-2, Strain:SARS-CoV-2_S1, Subunit,, His, Tag,, Baculovirus-Insect, Cells, Expression: HEK293, SinoCatalog: 40591-V08B1
VNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARAHHHHHHHHHH

SEQ ID NO: 50 - protein, 681 aa, SARS coronavirus Tor2; identical to SEQ ID NO: 49
  misc_feature (1)..(681): Antigen: SARS-CoV-2_S1,(HisTag), Virus: SARS-CoV-2, Strain:2019-nCoV, Expression: HEK293, SinoCatalog: 40591-V08H

SEQ ID NO: 51 - protein, 904 aa, SARS coronavirus Tor2
  misc_feature (1)..(904): Antigen: SARS-CoV-2_S1,(mFcTag), Virus: SARS-CoV-2, Strain:2019-nCoV, Expression: HEK293, SinoCatalog: 40591-V05H1
VNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARADDDDKAVPRDSGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 52 - protein, 457 aa, SARS coronavirus Tor2
  misc_feature (1)..(457): Antigen: SARS-CoV-2_S1-RBD, Virus: SARS-CoV-2, Strain:2019-nCoV, Expression: HEK293, SinoCatalog: 40592-V05H
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFADDDDKAVPRDSGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 53 - protein, 1209 aa, SARS coronavirus Tor2
  misc_feature (1)..(1209): Antigen: SARS-CoV-2_S1+S2, Virus: SARS-CoV-2, Strain:2019-nCoV, Expression: Insect, Cells, SinoCatalog: 40589-V08B1
VNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPAHHHHHHHHHH

SEQ ID NO: 54 - protein, 539 aa, SARS coronavirus Tor2
  misc_feature (1)..(539): Antigen: SARS-CoV-2_S2, Virus: SARS-CoV-2, Strain:2019-nCoV, Expression: Insect, Cells, SinoCatalog: 40590-V08B
SVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPAHHHHHHHHHH

SEQ ID NO: 55 - protein, 234 aa, SARS coronavirus Tor2
  misc_feature (1)..(234): Antigen: SARS-CoV-2, (2019-nCoV), Spike, RBD-His, Recombinant, Protein,, Baculovirus-Insect, Cells, Virus: SARS-CoV-2, Strain:2019-nCoV, Expression: Insect, Cells, SinoCatalog: 40592-V08B
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFAHHHHHHHHHH

SEQ ID NO: 56 - protein, 223 aa, SARS coronavirus Tor2; identical to SEQ ID NO: 4
  misc_feature (1)..(223): Antigen: SARS-CoV-2, (2019-nCoV), Spike, RBD-His, Recombinant, Protein,, HEK293, Virus: SARS-CoV-2, Strain:2019-nCoV, Uniprot/Genbank: , Expression: HEK293, SinoCatalog: 40592-V08H

SEQ ID NO: 57 - protein, 223 aa, SARS coronavirus Tor2; identical to SEQ ID NO: 4
  misc_feature (1)..(223): Antigen: SARS-CoV-2, (2019-nCoV), Spike, RBD-rFc, Recombinant, Protein, Virus: SARS-CoV-2, Strain:2019-nCoV, Uniprot/Genbank: , Expression: HEK293, SinoCatalog: 40592-V31H

SEQ ID NO: 58 - protein, 350 aa, SARS coronavirus WH20
  misc_feature (1)..(350): Antigen: SARS-CoV_PLpro, Virus: SARS-CoV, Strain:SARS, Uniprot/Genbank: AAX16193.1, Expression: E., coli, SinoCatalog: 40524-V08E
EVKTIKVFTTVDNTNLHTQLVDMSMTYGQQFGPTYLDGADVTKIKPHVNHEGKTFFVLPSDDTLRSEAFEYYHTLDESFLGRYMSALNHTKKWKFPQVGGLTSIKWADNNCYLSSVLLALQQLEVKFNAPALQEAYYRARAGDAANFCALILAYSNKTVGELGDVRETMTHLLQHANLESAKRVLNVVCKHCGQKTTTLTGVEAVMYMGTLSYDNLKTGVSIPCVCGRDATQYLVQQESSFVMMSAPPAEYKLQQGTFLCANEYTGNYQCGHYTHITAKETLYRIDGAHLTKMLEYKGPVTDVFYKETSYTTTIKPVSYKLDGVTYTEIEPKLDGYYKKDNAYYTEQPID

SEQ ID NO: 59 - protein, 665 aa, SARS coronavirus WH20
  misc_feature (1)..(665): Antigen: SARS-CoV_S1,(HisTag), Virus: SARS-CoV, Strain:SARS, Uniprot/Genbank: AAX16192.1, Expression: Insect, Cells, SinoCatalog: 40150-V08B1
SDLDRCTTFDDVQAPNYTQHTSSMRGVYYPDEIFRSDTLYLTQDLFLPFYSNVTGFHTINHTFDNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDTWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCSFGGVSVITPGTNASSEVAVLYQDVNCTDVSTAIHADQLTPAWRIYSTGNNVFQTQAGCLIGAEHVDTSYECDIPIGAGICASYHTVSLLRAHHHHHHHHHH

SEQ ID NO: 60 - protein, 233 aa, SARS coronavirus WH20
  misc_feature (1)..(233): Antigen: SARS-CoV_S1-RBD, (HisTag), Virus: SARS-CoV, Strain:SARS, Uniprot/Genbank: AAX16192.1, Expression: Insect, Cells, SinoCatalog: 40150-V08B2
RVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFAHHHHHHHHHH

SEQ ID NO: 61 - protein, 446 aa, SARS coronavirus WH20
  misc_feature (1)..(446): Antigen: SARS-CoV_S1-RBD,rFcTag, Virus: SARS-CoV, Strain:SARS, Uniprot/Genbank: AAX16192.1, Expression: Insect, Cells, SinoCatalog: 40150-V31B2
RVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFASKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK